(19) United States
(12) Patent Application Publication
(10) Pub. No.: US 2010/0210545 A1
(43) Pub. Date: Aug. 19, 2010

(54) PREVENTIVE/REMEDY FOR CANCER

(76) Inventors: Shinsuke Araki, Ibaraki (JP); Akira Hori, Ibaraki (JP); Yuusuke Nakayama, Ibaraki (JP)

Correspondence Address: EDWARDS ANGELL PALMER & DODGE LLP, P.O. BOX 55874, BOSTON, MA, 02205, US

(21) Appl. No.: 12/738,287

(22) PCT Filed: Oct. 15, 2008
(86) PCT No.: PCT/JP2008/069079

§ 371 Date: Apr. 15, 2010

(30) Foreign Application Priority Data
Oct. 15, 2007 (JP) 2007-268433

(51) Int. Cl. A61K 38/16; C12Q 1/02

(52) U.S. Cl. 514/12; 435/29

(57) ABSTRACT

The present invention provides identification of TRAIL signal activator sensitivity markers in cancer cells, and a method of diagnosing the sensitivity to TRAIL signal activator in a cancer patient using the markers. Tailor-made medical service of administering a TRAIL signal activator to a TRAIL signal activator sensitive cancer patient is provided.

The present invention provides a preventive or remedy agent for cancer for a TRAIL signal activator sensitive patient, the agent comprising a TRAIL signal activator, wherein the patient is screened by using the fluctuation in the expression or activity of TRAIL signal activator sensitivity markers in a sample collected from a test subject, as an index. As the TRAIL signal activator sensitivity markers, AIM1, STK17B, LOC93349, CASP8 and the like may be mentioned.

### TECHNICAL FIELD

The present invention relates to a method of diagnosing the sensitivity to a TRAIL signal activator in a cancer patient, a preventive/remedy agent for cancer comprising a TRAIL signal activator, which agent is administered to a TRAIL signal activator sensitive patient screened by the method, a preventive/remedy agent for cancer comprising a regulating agent for a molecule which influences the sensitivity to a TRAIL signal activator, and a method of screening a substance having an anticancer effect, the method comprising employing the inhibition/activation of a molecule which influences the sensitivity to a TRAIL signal activator, as an index.

### BACKGROUND OF THE INVENTION

TRAIL (TNF related apoptosis inducing ligand), which belongs to the TNF family, induces apoptosis by binding to a TRAIL receptor. Known members of the TRAIL receptors include TRAILR1 (also referred to as TNFRSF10A, DR4, APO2 or the like) and TRAILR2 (also referred to as TNFRSF10B, DR5 or the like), which have a death domain and transduce apoptosis signal, and decoy receptors DcR1 (also referred to as TNFRSF10C, TRAILR3, LIT, TRID or the like) and DcR2 (also referred to as TNFRSF10D, TRUNDD, or TRAILR4), which do not transduce apoptosis signal, and a soluble receptor Osteoprotegerin (also referred to as OPG, TNFRSF11B, or OCIF) which has no membrane-bound domain. Since TRAIL, agonist antibodies to TRAILR1, and agonist antibodies to TRAILR2 induce apoptosis in cancer cells which express TRAIL receptors having a death domain, clinical trials on their use as remedy agents for cancer are being conducted (Non-Patent Document 1).

When an agent which activates the TRAIL signal of TRAIL, agonist antibodies to TRAILR1 or TRAILR2, and the like (TRAIL signal activator) binds to a TRAIL receptor having a death domain, a death-inducing signaling complex (DISC) is formed by Fas associated death domain (FADD) and pro-caspase-8, and activation of caspase 8 occurs, which is the initial reaction of the caspase cascade. There are known two signal transduction paths downstream to the activation (Non-Patent Document 1). The first is a path in which caspase-8 directly activates executioner type caspase-3, -6 and -7, thereby inducing apoptosis, while the other is a path in which active type caspase-8 cleaves Bid, which belongs to the Bcl-2 family, and the cleaved Bid is transported to mitochondria to enhance membrane permeability, thereby indirectly activating the executioner type caspase-3, -6 and -7. As a result of the transport of Bid to mitochondria, cytochrome c, AIF or Smac/DIBLO, which activates the caspase cascade through the activation of Apaf-1, is released from the mitochondria to activate the executioner type caspase-3, -6 and -7, whereby apoptosis occurs.

Meanwhile, it has been found by the studies on TRAIL signaling that there exist cancer cells which are resistant to TRAIL-induced apoptosis, and to the present, it has been reported that abnormality in various proteins which regulate the TRAIL receptors and the apoptosis downstream thereof, such as reduced expression of a receptor containing a death domain (TRAILR1 or TRAILR2), high expression of a decoy receptor, mutation and reduced expression of caspase-8, enhanced expression of c-FLIP (cellular FADD-like interleukin-1β-converting enzyme inhibitory protein) which inhibits the activation of caspase-8 in DISC, high expression of Bcl-2 and Bcl-XL which inhibit the release of cytochrome c and the activation of Apaf-1, enhanced expression of the IAP (inhibitor of apoptosis proteins) family (XIAP, survivin, or the like) which inhibits caspase, and the like; and the proliferation of MAPK, NFκB or the like, activation of anti-apoptosis signal, as well as glycosyltransferases GALNT14 and GALNT3 which glycosylate TRAILR1 and TRAILR2, and fucosyltransferases FUT6 and FUT3 which fucosylate TRAILR1 and TRAILR2, are involved with the TRAIL-resistance (Non-Patent Documents 2 to 5).

However, nothing is known so far about a biomarker which is capable of determining conveniently and highly accurately as to whether or not a cancer cell is sensitive to TRAIL-induced apoptosis, in other words, as to whether or not a cancer patient is sensitive to a TRAIL signal activator; that is, a TRAIL signal activator sensitivity marker.

[Non-Patent Document 1] Duiker, E. W., et al., Eur. J. Cancer, 2006, 42:2233-40
[Non-Patent Document 2] Caroline M. M., et al., Drug Resist. Updat., 2004, 7:345-58

### [Non-Patent Document 3] Zhang, L. and Fang, B., Cancer Gene Ther., 2005, 12:228-37

### [Non-Patent Document 4] Pasquini, L., et al., Cancer Ther., 2006, 4:47-72

[Non-Patent Document 5] Wagner K. W., et al., Nat. Med., 2007, 13:1070-7

### SUMMARY OF THE INVENTION

Therefore, it is an object of the present invention to provide a method of diagnosing the sensitivity to TRAIL signal activator in a cancer patient by identifying TRAIL signal activator sensitivity markers in cancer and using the markers, thus providing tailor-made type medical service whereby a TRAIL signal activator is administered to a cancer patient who is TRAIL signal activator sensitive. It is another object of the invention to provide a method of augmenting the sensitivity to TRAIL signal activator by identifying factors related to the sensitivity/insensitivity to TRAIL signal activator, and targeting the factors. It is still another object of the invention to provide a method of screening a substance having an anticancer effect by using the regulation of expression/function of the factors related to the sensitivity/insensitivity to a TRAIL signal activator, as an index.

In order to achieve the above-mentioned objects, the present inventors carried out extensive investigations, and as a result, they succeeded for the first time in identifying various TRAIL signal activator sensitivity markers, and established a diagnosis method which can determine with high accuracy the sensitivity to a TRAIL signal activator in a cancer patient using such markers. Furthermore, the present inventors found that the sensitivity to TRAIL signal activator in cancer cells can be changed by regulating the expression or function of such markers (hereinafter, may also be referred to as “TRAIL signal activator sensitivity-related factor”), and demonstrated that the regulator of the markers is useful per se as a preventive/remedy agent for cancer.

The inventors carried out further investigation based on these findings, and thereby completed the invention.

That is, the present invention provides:

[1] a preventive or remedy agent for cancer in a TRAIL signal activator sensitive patient, the agent containing a TRAIL signal activator, in which the patient is screened on the basis of an index indicating a fluctuation in the expression or activity of a TRAIL signal activator sensitivity marker in a sample collected from a test subject;
[2] the agent as described in [1], in which patient who is sensitive to TRAIL signal activator is selected on the basis of an index including conditions where (i) the expression or activity of AIM1 is not promoted and (ii) the expression or activity of at least 2 selected from the group consisting STK17B, LOC93349, CASP8, SP110, NOD27, and RHOBTB3 is promoted, in sample collected from a test subject;
[3] the agent as described in [1], in which patient who is sensitive to TRAIL signal activator is selected on the basis of an index including conditions where the expression or activity of AIM1 is decreased, and the expression or activity of STK17B and/or LOC93349 and/or CASP8 is promoted, in sample collected from a test subject;
[4] the agent as described in [1], in which patient who is sensitive to TRAIL signal activator is selected on the basis of an index including condition where the expression or activity of STK17B is promoted, in sample collected from a test subject;
[5] the agent as described in [1], wherein the TRAIL signal activator sensitivity marker includes AIM1, STK17B, LOC93349 and CASP8, and in the case where:

(1) the expression level or activity of AIM1, STK17B, LOC93349 and CASP8 in two or more known TRAIL signal activator sensitive cancer cells and two or more known TRAIL signal activator insensitive cancer cells, is measured, and

(2)(i) from the relationship between the expression level or activity of AIM1 in breast cancer or colon cancer cells, among the TRAIL signal activator sensitive cancer cells and TRAIL signal activator insensitive cancer cells as measured in (1), the value of a relative expression level or relative activity which can be used to accurately determine the presence or absence of the sensitivity to a TRAIL signal activator in a cancer cell, is taken as the reference value; and

(ii) from the relationship between the expression level or activity of STK17B, LOC93349 or CASP8 in the TRAIL signal activator sensitive cancer cells and TRAIL signal activator insensitive cancer cells as measured in (1), and the TRAIL signal activator sensitivity, the value of a relative expression level or relative activity which can be used to accurately determine the TRAIL signal activator sensitivity of a cancer cell, is taken as the upper reference value, while the value of a relative expression level or relative activity which can be used to accurately determine the TRAIL signal activator insensitivity of a cancer cell, is taken as the lower reference value,

a test subject corresponding to the following (a) or (b) is screened as a TRAIL signal activator sensitive patient:

(a) in case the test subject is a patient suffering from breast cancer or colon cancer, the expression level or activity of AIM1 in a sample collected from the test subject is less than the reference value, and given that a +1 point is scored if the expression level or activity of STK17B, LOC93349 or CASP8 in the sample collected from the test subject is equal to or greater than the upper reference value; a zero point is scored if the amount of expression or activity is equal to or greater than the lower reference value and also less than the upper reference value; and a −1 point is scored if the expression level or activity is less than the lower reference value, the sum of the points for STK17B, LOC93349 and CASP8 is a positive value; and

(b) in case the test subject is a patient suffering from a cancer other than breast cancer and colon cancer, given that a +1 point is scored if the expression level or activity of STK17B, LOC93349 or CASP8 in the sample collected from the test subject is equal to or greater than the upper reference value; a zero point is scored if the expression level or activity is equal to or greater than the lower reference value and also less than the upper reference value; and a −1 point is scored if the expression level or activity is less than the lower reference value, the sum of the points for STK17B, LOC93349 and CASP8 is a positive value;

[6] the agent as described in [5], wherein the TRAIL signal activator sensitivity marker includes AIM1, STK17B, LOC93349 and CASP8, and in the case where:

(1) the expression level or activity of AIM1, STK17B, LOC93349 and CASP8 in 2 or more known TRAIL signal activator sensitive cancer cells and 2 or more known TRAIL signal activator insensitive cancer cells is measured,

(2)(i) using the expression level or activity of AIM1, STK17B, LOC93349 and CASP8 in one cell arbitrarily selected from TRAIL signal activator sensitive cancer cells and TRAIL signal activator insensitive cancer cells (reference cancer cell) as measured in (1) as 1.0, the relative expression level or relative activity of AIM1, STK17B, LOC93349 and CASP8 in other cancer cells is calculated,

(ii) the relationship between the relative expression level (two places of decimals) or relative activity (two places of decimals) of AIM1 in other cells as measured in (1) and the presence or absence of TRAIL signal activator sensitivity in said cells is compared, the highest relative expression level A or relative activity A in the TRAIL signal activator sensitive cancer cell group is selected, relative expression level B or relative activity B in the TRAIL signal activator insensitive cancer cell group, which is greater than the relative expression level A or relative activity A and the nearest thereto, and the value of relative expression level B or relative activity B less the second place of decimal is taken as the reference value,

(iii) the relationship between the relative expression level (two places of decimals) or relative activity (two places of decimals) of STK17B, LOC93349 and CASP8 in other cells as measured in (1) and the presence or absence of TRAIL signal activator sensitivity in said cells is compared, 80% value of the median value of the relative expression level or relative activity in the TRAIL signal activator sensitive cancer cell group is taken as the upper reference value, and 120% value of the median value of the relative expression level or relative activity in the TRAIL signal activator insensitive cancer cell group is taken as the lower reference value,

a test subject corresponding to the following (a) or (b) is screened as a TRAIL signal activator sensitive patient:

(a) in case the test subject is a patient suffering from breast cancer or colon cancer, the expression level or activity of AIM1 in a sample collected from the test subject is less than the reference value, and given that a +1 point is scored if the expression level or activity of STK17B, LOC93349 or CASP8 in the sample collected from the test subject is equal to or greater than the upper reference value; a zero point is scored if the amount of expression or activity is equal to or greater than the lower reference value and also less than the upper reference value; and a −1 point is scored if the expression level or activity is less than the lower reference value, the sum of the points for STK17B, LOC93349 and CASP8 is a positive value; and

(b) in case the test subject is a patient suffering from a cancer other than breast cancer and colon cancer, given that a +1 point is scored if the expression level or activity of STK17B, LOC93349 or CASP8 in the sample collected from the test subject is equal to or greater than the upper reference value; a zero point is scored if the expression level or activity is equal to or greater than the lower reference value and also less than the upper reference value; and a −1 point is scored if the expression level or activity is less than the lower reference value, the sum of the points for STK17B, LOC93349 and CASP8 is a positive value;

[7] the agent as described in [6], wherein the expression levels of AIM1, STK17B, LOC93349 and CASP8 are the expression levels of mRNAs encoding them, the relative expression level is the relative value to the expression level in COLO205 cell line, the reference value for AIM1 is 2.2, the upper reference value is 1.036 and the lower reference value is 0.514 for STK17B, the upper reference value is 0.666 and the lower reference value is 0.211 for LOC93349, and the upper reference value is 0.833 and the lower reference value is 0.519 for CASP8;
[8] the agent as described in [1], wherein the sample is cancer cells, cancer tissue, blood, serum, blood plasma or urine;
[9] the agent as described in any one of [1] to [8], wherein the TRAIL signal activator is TRAIL, a TRAILR1 agonist compound, or a TRAILR2 agonist compound;
[10] a method of diagnosing the sensitivity to a TRAIL signal activator in a patient suffering from cancer, the method comprising examining the expression or activity of STK17B, LOC93349 and CASP8 in a sample collected from the patient;
[11] the method as described in [10], wherein the method is a method of diagnosing the sensitivity to a TRAIL signal activator in a patient suffering from breast cancer or colon cancer, and the method comprises further examining the expression or activity of AIM1 in a sample collected from the patient;
[12] the method as described in [10], wherein the TRAIL signal activator is TRAIL, a TRAILR1 agonist compound or a TRAILR2 agonist compound;
[13] a diagnostic agent for the sensitivity to a TRAIL signal activator in a patient suffering from cancer, the diagnostic agent comprising a reagent which can detect the expression or activity of STK17B, LOC93349 and CASP8;
[14] the diagnostic agent as described in [13], wherein the diagnostic agent further comprises a reagent which can detect the expression or activity of AIM1, and is for a patient suffering from breast cancer or colon cancer;
[15] a preventive or remedy agent for cancer, comprising an AIM1 inhibitor;
[16] a method of screening a compound having an anticancer effect, or a salt thereof, the method comprising employing the inhibition of AIM1 as an index;
[17] a preventive or remedy agent for cancer, comprising an activator of STK17B, LOC93349, SP110, NOD27 or RHOBTB3;
[18] a method of screening a compound having an anticancer effect, or a salt thereof, the method comprising employing the activation of STK17B, LOC93349, SP110, NOD27 or RHOBTB3 as an index;

Furthermore, the present invention provides:

[19] a method of preventing or treating cancer, comprising administering an effective amount of a TRAIL signal activator to a TRAIL signal activator sensitive patient screened using, as an index, variation in the expression or activity of a TRAIL signal activator sensitive marker in samples taken from test subjects;
[20] a method of preventing or treating cancer, comprising administering an effective amount of an AIM1 inhibitor to a mammal;
[21] a method of preventing or treating cancer, comprising administering an effective amount of an STK17B, LOC93349, SP110, NOD27 or RHOBTB3 activator to a mammal;
[22] use of a TRAIL signal activator for the production of an agent for preventing or treating cancer in a TRAIL signal activator sensitive patient, wherein the patient is screened using, as an index, variation in the expression or activity of a TRAIL signal activator sensitive marker in samples taken from test subjects;
[23] use of an AIM1 inhibitor for the production of an agent for preventing or treating cancer; and
[24] use of an STK17B, LOC93349, SP110, NOD27 or RHOBTB3 activator for the production of an agent for preventing or treating cancer.

As an examination of the expression or activity of the TRAIL signal activator sensitivity markers of the invention in a cancer patient is carried out, rapid and convenient diagnosis of the sensitivity to TRAIL signal activator is made possible. Also, since the preventive/remedy agent for cancer of the invention, which contains a TRAIL signal activator, is selectively administered to a cancer patient who is sensitive to TRAIL signal activator, cancer can be prevented and/or remedied more effectively. Furthermore, since the regulator of the TRAIL signal activator sensitivity related factor of the invention can increase the sensitivity to TRAIL signal activator in a cancer patient, cancer can be effectively prevented and/or remedied, for example, by using the regulator in combination with a TRAIL signal activator.

### DETAILED DESCRIPTION OF THE INVENTION

### 1. Preventive/Remedy Agent for Cancer Containing Trail Signal Activator

The present invention provides a preventive or remedy agent for cancer for TRAIL signal activator sensitive patients, the agent comprising a TRAIL signal activator. Here, the “TRAIL signal activator” is not particularly limited as long as it is a substance which can activate the signal transduction from TRAIL through TRAILR1 or TRAILR2, finally to induce apoptosis in cells. However, the TRAIL signal activator is preferably a substance which can bind to TRAILR1 or TRAILR2 and transduce signal to FADD and pro-caspase-8, to trigger the activation of caspase-8, and may be exemplified by TRAIL, agonist compounds of TRAILR1 or TRAILR2, or the like. Details of the respective TRAIL signal activator will be described later.

The ‘sensitivity to TRAIL signal activator’ means that transduction of TRAIL signal in cancer cells is activated under the action of a TRAIL signal activator, so that apoptosis is induced in cancer cells to an extent that is effective in the preventive and/or remedy for cancer.

In the specification, the term ‘patient’ refers to those suffering from cancer, or those having a risk of developing cancer in the future, as cells are in the process of malignant alteration. In addition, ‘patient’ is not only referring to human, but also includes other mammals (e.g., simian, bovine, horse, canine, feline, sheep, goat, hamster, guinea pig, rabbit, rat, mouse, etc.), but preferably human. The type of cancer is not particularly limited, but examples of cancers include for example, breast cancer (e.g., invasive breast cancer, noninvasive breast cancer, inflammatory breast cancer, etc.), prostate cancer (e.g., hormone-dependent prostate cancer, hormone-independent prostate cancer, etc.), pancreas cancer (e.g., pancreas cancer, etc.), stomach cancer (e.g., papillary adenocarcinoma, mucous gland carcinoma, adenosquamous carcinoma, etc.), lung cancer (e.g., non-small cell lung cancer, small cell lung cancer, malignant mesothelioma, etc.), colon cancer (e.g., gastrointestinal stromal tumor, etc.), rectal cancer (e.g., gastrointestinal stromal tumor, etc.), large intestine cancer (e.g., familial colon cancer, hereditary nonpolyposis colon cancer, gastrointestinal stromal tumor, etc.), small intestine cancer (e.g., non-Hodgkin's lymphoma, gastrointestinal stromal tumor, etc.), esophagus cancer, duodenal cancer, tongue cancer, pharynx cancer (e.g., nasopharyngeal cancer, oropharynx cancer, hypophrynx cancer etc.), salivary gland cancer, brain tumor (e.g., pineal astrocytoma, pilocytic astrocytoma, diffuse astrocytoma, anaplastic astrocytoma, etc.), neurilemmonma, liver cancer (e.g., primary hepatic cancer, extrahepatic bile duct cancer, etc.), kidney cancer (e.g., renal cell cancer, transitional cell cancer of the renal pelvis and ureter, etc.), bile duct cancer, endometrial cancer, uterine cervix cancer, ovary cancer (e.g., epithelial ovarian cancer, extragonadal germ cell tumor, ovarian germ cell tumor, ovarian low-malignant potential tumor, etc.), bladder cancer, urethral cancer, skin cancer (e.g., intraocular (eye) melanoma, Merkel cell carcinoma, etc.), angioma, malignant lymphoma, malignant melanoma, thyroid cancer (e.g., medullary cancer, etc.), parathyroid cancer, nasal cancer, paranasal cancer, bone tumor, (e.g., osteosarcoma, Ewing's tumor, uterine sarcoma, soft tissue sarcoma, etc.), hemangiofibroma, retinal sarcoma, penis cancer, testicular tumor, solid tumor in children (e.g., Wilm's tumor, childhood kidney tumor, etc.), Kaposis's sarcoma, AIDS-associated (related) Kaposis's sarcoma, tumor of maxillary sinus, fibrous histiocytoma, leiomyosarcoma, rhabdomyosarcoma, leukemia (e.g., acute myeloid leukemia, acute lymphoblastic leukemia, etc.), and the like, preferably breast cancer, large intestine cancer, lung cancer, stomach cancer, pancreas cancer, prostate cancer, ovary cancer, blood cancer.

The TRAIL signal activator sensitive patient who serves as the subject of administration of the preventive/remedy agent for cancer of the invention comprising a TRAIL signal activator, is characterized in being screened by employing the presence or absence of any fluctuation in the expression or activity of one or more TRAIL signal activator sensitivity marker in a sample collected from a test subject, as an index. Heretofore, since TRAIL signal activator sensitive biomarkers have not been known, it is impossible to use the TRAIL signal activator for defined subjects of administration. Therefore, the invention also provides, for the first time, TRAIL signal activator sensitivity markers.

Examples of ‘TRAIL signal activator sensitivity marker’ in the invention include AIM1, STK17B, LOC93349, CASP8, SP110, NOD27, and RHOBTB3. AIM1, STK17B, LOC93349, CASP8, SP110, NOD27, and RHOBTB3 are each a generic symbol established by NCBI (National Center for Biotechnology Information). However, when simply described as ‘AIM1’, ‘STK17B’, ‘LOC93349’, ‘CASP8’, ‘SP110’, ‘NOD27’, and ‘RHOBTB3’ in the specification, the symbol may indicate a gene, a protein encoded by the gene, or may indicate both inclusively, depending on the context.

AIM1 gene is a gene containing the same or substantially the same base sequence as the base sequence represented by SEQ ID NO: 1, which is known to be related to tumor suppression in a malignant melanoma (Proc. Natl. Acad. Sci. USA, 94: 3229-34, 1997). It has been suggested that the gene has 12 membrane-spanning domains (βγ-crystalline motif), and is localized in cell membrane.

STK17B gene is a gene containing the same or substantially the same base sequence as the base sequence represented by SEQ ID NO: 3, which encodes serine/threonine kinase which is suggested to be related to apoptosis signal transduction (J. Biol. Chem., 273 (44): 29066-71, 1998).

LOC93349 gene is a gene containing the same or substantially the same base sequence as the base sequence represented by SEQ ID NO: 5, which encodes a protein having unknown function (Nat. Genet., 36 (1): 40-5, 2004).

CASP8 gene is a gene containing the same or substantially the same base sequence as the base sequence represented by SEQ ID NO: 7, which encodes caspase-8 located in the top of the caspase cascade (Duiker E. W. et al., Eur. J. Cancer, 2006, 42: 2233-40).

SP110 gene is a gene containing the same or substantially the same base sequence as the base sequence represented by SEQ ID NO: 9, which encodes a protein which is suggested to be a constituent component of nuclear body and functions as transcriptional coactivator of a nuclear hormone receptor (Mol. Cell. Biol., 20 (16): 6138-46, 2000).

NOD27 gene is a gene containing the same or substantially the same base sequence as the base sequence represented by SEQ ID NO: 11, which encodes a protein having unknown function containing a domain having homology to Apaf-1 (nucleotide binding polymeric type domain) (Biochem. Biophys. Res. Commun. 302 (3): 575-580, 2003).

RHOBTB3 gene is a gene containing the same or substantially the same base sequence as the base sequence represented by SEQ ID NO: 13, and encodes proteins which are members of the subfamily of Rho GTPase. However, this gene is upregulated by several cancer cell lines, and thus it is suggested that the proteins are involved in carcinogenesis (Gene, 298 (2):147-57, 2002).

Here, the “gene containing substantially the same base sequence as the base sequence represented by “SEQ ID NO: n (n=1, 3, 5, 7, 9, 11 or 13)” means a naturally occurring human gene which is not completely identical to the base sequence represented by SEQ ID NO: n, but has the same function, such as a splicing variant of a human gene encoding a human cDNA comprising the base sequence represented by SEQ ID NO: n, an allelic variant (for example, allele frequency being less than 1%), a gene polymorphism (for example, minor allele frequency of not less than 1%), or the like. For example, CASP8 is known to have four splicing variants, and the base sequence of variant A (Refseq Accession No. NM_{—}001228.4), which is the longest among them, is represented by SEQ ID NO: 7, while there are also variant B registered as Refseq Accession No. NM_{—}033355.2, variant C registered as Refseq Accession No. NM_{—}033356.2, and variant E registered as Refseq Accession No. NM_{—}033358.2. Also, SP110 is known to have three splicing variants, and the base sequence of variant C (Refseq Accession No. NM 80424.1), which is the longest among them, is represented by SEQ ID NO: 9, while there are also variant A registered as Refseq Accession No. NM_{—}004509.2, and variant B registered as Refseq Accession No. NM 004510.2.

The foregoing variant or polymorphic gene usually contains a base sequence having at least about 90% homology, preferably at least about 95% homology, more preferably at least about 97% homology, particularly preferably at least about 98% homology, and most preferably at least about 99% homology. Furthermore, homology of the base sequences according to the present specification can be calculated using a homology calculation algorithm, NCBI BLAST (National Center for Biotechnology Information Basic Local Alignment Search Tool), under the following conditions (expected value=10; gap allowed; filtering ═ON; match score=1; mismatch score=−3). As other algorithms for determining the homology of a base sequence, for example, the algorithm described in Karlin et al., Proc. Natl. Acad. Sci. USA, 90:5873-5877 (1993) [this algorithm has been incorporated into NBLAST and XBLAST programs (version 2.0) (Altschul et al., Nucleic Acids Res., 25:3389-3402 (1997))]; the algorithm described in Needleman et al., J. Mol. Biol., 48:444-453 (1970) [this algorithm has been incorporated into the GAP program in the GCG software package]; the algorithm described in Myers and Miller, CABIOS, 4:11-17 (1988) [this algorithm has been incorporated into the ALIGN program (version 2.0), a part of the CGC sequence alignment software package]; the algorithm described in Pearson et al., Proc. Natl. Acad. Sci. USA, 85:2444-2448 (1988) [this algorithm has been incorporated into the FASTA program in the GCG software package]; and the like may be mentioned, and these can also be likewise used favorably.

As discussed above, the preventive/remedy agent for cancer of the invention containing a TRAIL signal activator can also be widely applied to non-human mammals. Therefore, it is apparent that the TRAIL signal activator sensitivity marker genes according to the invention include the human genes as well as orthologues thereof for other mammals. In this case, the homology between the orthologues and human genes is not particularly limited, and higher homology would be preferable. For example, the homology may be at least about 50%, preferably at least about 60%, more preferably at least about 70%, even more preferably at least about 80%, and particularly preferably at least about 90%. The sequence information for the orthologues of other mammals can be obtained by performing a search from databases for the genome and/or cDNA of mammals other than human being, using BLAST or FASTA, by querying the base sequence itself represented by SEQ ID NO: n or the accession number in public databases (for example, the Refseq Accession Number of AIM1 is NM_{—}001624.2, that of STK17B is NM_{—}002446.2, that of LOC93349 is NM_{—}138402.4, that of CASP8 is NM_{—}001228.4, that of SP110 is NM_{—}080242.1, that of NOD27 is NM_{—}032206.3, and that of RHOBTB3 is NM_{—}014899.3), or by accessing the information of Mammalian Orthology for the data hit by performing a search in, for example, the Mouse Genome Informatics (http://www.informatics.jax.org/) provided by the Jackson Laboratory, using the accession number or gene symbol/gene name as the keyword. For example, in the case of AIM1 gene, a mouse orthologue registered as Refseq Accession No. BC059272.1 in GenBank, a chimpanzee orthologue registered as Refseq Accession No. XM_{—}518660.2 in GenBank, and a dog orthologue registered as Refseq Accession No. XM_{—}532248.2 in GenBank are known.

The “expression (level)” of the TRAIL signal activator sensitivity marker includes both (1) the expression (level) of mRNAs of AIM1, STK17B, LOC93349, CASP8, SP110, NOD27 or RHOBTB3 genes, and (2) the expression (level) of proteins from those mRNAs. On the other hand, the “activity” of the TRAIL signal activator sensitivity marker means the physiological activity of AIM1, STK17B, LOC93349, CASP8, SP110, NOD27 or RHOBTB3 proteins, and for example, the kinase (self-phosphorylation and protein phosphorylation) activity in the case of STK17B, and the protease activity in the case CASP8 may be mentioned.

The expression of the TRAIL signal activator sensitivity marker can be examined by measuring the amount of the mRNA or protein of the marker contained in a sample collected from a test subject. Also, the activity of a TRAIL signal activator sensitivity marker can be examined by measuring the physiological activity of the marker protein contained in the sample, using methods known per se with regard to the respective proteins.

The sample as used herein is collected from the mammal described above as the test subject, and the sample is not particularly limited as long as it contains a gene product (for example, mRNA, protein, etc.) as the object of detection. For example, in addition to cancer cells or cancer tissue samples obtained by biopsy, there may be mentioned body fluids such as whole blood, cell fractions from blood (may include circulating tumor cells (CTC)), blood plasma, serum, lymph fluid, cerebrospinal fluid, semen, urine, sweat, saliva, joint fluid and the like, or fractions thereof, mucosa, and the like. When considering the fact that the TRAIL signal activator sensitivity markers have been extracted from the comparison of expression in various TRAIL signal activator sensitive and insensitive cancer cells, it is preferable to use cancer cells or cancer tissues; however, since the process of collection can be carried out rapidly and conveniently, and is less invasive to animals, it is also preferable to use blood or fractions thereof.

According to the invention, it is desirable to examine the expression or activity of at least one selected from the aforementioned seven TRAIL signal activator sensitivity markers. Preferably, a method of using any two or more, more preferably three or more, and even more preferably four or more, among the seven species, as the object of measurement, may be mentioned.

Particularly preferably, a method of using three markers, namely, STK17B, LOC93349 and CASP8, may be mentioned, and in the case where the test subject is a patient suffering from breast cancer or colon cancer, it is preferable to further use AIM1 as the object of measurement.

Expression of the TRAIL signal activator sensitivity marker genes in a sample collected from a test subject mammal can be examined by preparing an RNA (for example, whole RNA, mRNA) fraction from the sample, and detecting transcription products of the marker genes contained in the fraction. The preparation of the RNA fraction can be carried out using a known technique such as a guanidine-CsCl ultracentrifugation method, an AGPC method or the like, but high purity whole RNA can be prepared rapidly and conveniently from a trace amount of sample, using a commercially available RNA extraction kit (for example, RNeasy Mini Kit manufactured by QIAGEN, etc.). As the means for detecting the transcription products of the TRAIL signal activator sensitivity marker genes in the RNA fraction, for example, a method of using hybridization (Northern blotting, dot blotting, DNA chip analysis, etc.), a method of using PCR(RT-PCR, competitive PCR, real time PCR, etc.), or the like may be mentioned. From the viewpoint that the fluctuation in expression of TRAIL signal activator sensitivity marker genes can be detected from a trace amount of sample rapidly and conveniently with good quantitative capability, a quantitative PCR method such as competitive PCR, real time PCR or the like is preferred, while from the viewpoint that the fluctuation in the expression of a plurality of marker genes can be collectively detected, and the quantitative capability can be improved by the selection of the detection method, DNA chip analysis is preferred.

In the case of performing Northern blot or dot blot hybridization, detection of a TRAIL signal activator sensitivity marker gene can be performed using a nucleic acid (probe) which can be hybridized with the transcription product of the gene. Such nucleic acid may be exemplified by the transcription product of a TRAIL signal activator sensitivity marker gene, that is, a nucleic acid which can be hybridized, under highly stringent conditions, with a nucleic acid (sense strand=coding strand) having (i) a base sequence represented by SEQ ID NO: n (n=1, 3, 5, 7, 9, 11 or 13), or (ii) the base sequence of an orthologue of a human gene containing the base sequence of (i) above in an other mammal. The “highly stringent conditions” refer to the conditions under which a nucleic acid having a base sequence having at least about 80% complementarity, preferably at least about 90% complementarity, and more preferably at least about 95% complementarity, in the region overlapping with a nucleic acid having each of the base sequence represented by SEQ ID NO: n, and for example, there may be mentioned a hybridization reaction at 45° C. in 6×SSC (sodium chloride/sodium citrate) followed by washing once or more at 65° C. in 0.2×SSC/0.1% SDS after, or the like. Those ordinarily skilled in the art can easily adjust the conditions to desired stringency by appropriately altering the salt concentration in the hybridization solution, the temperature of the hybridization reaction, the probe concentration, the length of the probe, the number of mismatches, the duration of the hybridization reaction, the salt concentration of the washing solution, the temperature of the washing process, or the like. The nucleic acid may be a DNA or an RNA, or may also be a DNA/RNA chimera. Preferably, the nucleic acid may be a DNA.

The nucleic acid used as the probe may be double-stranded or single-stranded. In the case of being double-stranded, the nucleic acid may be a double-stranded DNA, a double-stranded RNA or a hybrid of DNA:RNA. In the case of a single-stranded nucleic acid, an antisense strand can be used. The length of the nucleic acid is not particularly limited as long as the nucleic acid can be specifically hybridized with a target nucleic acid, and for example, the length is at least about 15 bases, and preferably at least about 30 bases. The nucleic acid is preferably labeled by a labeling agent so as to enable detection/quantification of the target nucleic acid. As the labeling agent, for example, a radioisotope, an enzyme, a fluorescent material, a luminescent material or the like is used. The radioisotope may be exemplified by [¹²⁵I], [¹³¹I], [³H], [¹⁴C], [³²P], [³³P], [³⁵S] or the like. The enzyme is preferably an enzyme which is stable and has high specific activity, and for example, β-galatosidase, β-glucosidase, alkaline phosphatase, peroxidase, malate dehydrogenase or the like is used. The fluorescent material may be exemplified by fluorescamine, fluorescent isothiocyanate, a cyanine fluorescent dye or the like. The luminescent material may be exemplified by luminol, a luminol derivative, luciferin, lucigenin or the like. Furthermore, biotin-(strept)avidin may also be used for the binding between a probe and a labeling agent.

In the case of Northern hybridization, an RNA fraction prepared as described above is separated by gel electrophoresis, subsequently transferred to a membrane made of nitrocellulose, nylon, polyvinylidene fluoride or the like, and hybridized under the “highly stringent conditions” described above in a hybridization buffer solution containing a labeled probe prepared as described above, and then the amount of the label bound to the membrane is measured for each band by an appropriate method, whereby the expression level of each of the TRAIL signal activator sensitivity marker genes can be measured. In the case of dot blotting, a membrane spotted with an RNA fraction is subjected to a hybridization reaction (performed for each of the TRAIL signal activator sensitivity marker genes), and the amount of label of the spot is measured, whereby the expression level of each of the marker genes can be measured.

In the case of DNA chip analysis, for example, a cDNA to which an appropriate promoter such as T7 promoter has been introduced is synthesized from an RNA fraction prepared as described above by a reverse transcription reaction, and a cRNA is further synthesized using an RNA polymerase (here, a labeled cRNA is obtained using a mononucleotide labeled with biotin or the like, as a substrate). This labeled cRNA is subjected to a hybridization reaction by contacting the cRNA with a chip on which the probes are immobilized, and the amount of the label bound to each of the immobilized probes is measured, whereby the expression level of each of the TRAIL signal activator sensitivity marker genes can be measured. This method is advantageous in terms of rapidity and convenience, as the number of TRAIL signal activator sensitivity marker genes to be detected (therefore, probes being immobilized) increases.

According to another preferred embodiment, as a method of measuring the expression level of the TRAIL signal activator sensitivity marker genes, a quantitative PCR method is used. Quantitative PCR may be exemplified by competitive PCR or real time PCR.

As for the oligonucleotide set used as the primers in PCR, there is no particular limitation as long as an oligonucleotide set can be specifically hybridized with the sense strand (coding strand) and the antisense strand (non-coding strand), respectively, of each transcription product of the TRAIL signal activator sensitivity marker genes, and can amplify the DNA fragments lying between them. For example, there may be mentioned an oligo-DNA set in which each oligo-DNA has a length of about 15 to about 100 bases, preferably about 15 to about 50 bases, and which is designed to amplify DNA fragments of about 100 by to several kbp. More particularly, there may be mentioned a nucleic acid which can be hybridized, under highly stringent conditions, with a nucleic acid (sense strand) having (i) a base sequence represented by SEQ ID NO: n, or (ii) the base sequence of an orthologue of a human gene comprising the base sequence of (i) above in an other mammal, and a nucleic acid which can be hybridized, under highly stringent conditions, with a nucleic acid having a base sequence that is complementary to the base sequence of (i) or (ii) above (antisense strand). Here, the “highly stringent conditions” means the same as described above.

Competitive RT-PCR means a method of calculating the amount of a desired DNA by adding a known amount of different template nucleic acid which can be amplified by a primer set capable of amplifying the desired DNA, as a competitor to the reaction solution, thus to trigger an amplification reaction in a competitive manner, and comparing the amounts of amplification products. Therefore, in the case of competitive RT-PCR, in addition to the primer set described above, there is used a known amount of a competitor nucleic acid which can be amplified by the primer set, and after amplification, can be distinguished (for example, having a different amplification size, having a different electrophoresis pattern for the restriction enzyme treated fragments, etc.) from the amplification product of a target nucleic acid (that is, the transcription product of a TRAIL signal activator sensitivity marker gene). Since the target nucleic acid and the competitor nucleic acid undergo amplification in a competitive manner by taking the primers away from each other, the ratio of the amounts of amplification products reflects the ratio of the amounts of templates. The competitor nucleic acid may be a DNA or an RNA. If the competitor nucleic acid is a DNA, a cDNA may be synthesized through a reverse transcription reaction from an RNA fraction prepared as described above, and then PCR may be performed in the co-presence of the above-described primer set and a competitor. If the competitor nucleic acid is an RNA, a reverse transcription reaction may be performed by adding a competitor to the RNA fraction, and then PCR may be performed by adding the above-described primer set. In the latter case, since the efficiency of the reverse transcription reaction is also taken into account, the absolute amount of the original mRNA can be estimated.

Meanwhile, real time PCR is a method of monitoring the amount of amplification in real time by using a fluorescent reagent, and requires an apparatus having a thermal cycler and a fluorospectrophotometer integrated together. Such an apparatus is commercially available. There are a few methods depending on the fluorescent reagent used, and for example, an intercalator method, a TaqMan™ probe method, a Molecular Beacon method or the like may be mentioned. In any case, cDNA is synthesized by reverse transcription reaction from an RNA fraction prepared as described above, and then the primer set described above and a fluorescent reagent (probe), such as a reagent which emits fluorescence by binding to double-stranded DNA such as SYBR Green I, ethidium bromide or the like (intercalator); a nucleic acid which can be used as the probe (provided that the nucleic acid is hybridized with a target nucleic acid in the amplification region), which nucleic acid having its two terminals modified with a fluorescent material (for example, FAM, HEX, TET, FITC, etc.) and a quenching material (for example, TAMRA, DABCYL, etc.), respectively (TaqMan™ probe or Molecular Beacon probe); or the like, is added to the PCR reaction system. Since the intercalator binds to a synthesized double-stranded DNA and emits fluorescence when irradiated with an excitation light, the amount of the amplification product produced can be monitored by measuring the fluorescence intensity, and thereby the amount of the original template cDNA can be estimated. The TaqMan™ probe is an oligonucleotide having its two terminals modified with a fluorescent material and a quenching material, respectively, which oligonucleotide can be hybridized into the amplification region of a target nucleic acid. Although the probe is hybridized into the target nucleic acid during annealing, the probe does not emit fluorescence due to the presence of the quenching material, and emits fluorescence when the probe is degraded by the exonuclease activity of a DNA polymerase during the extension reaction, whereby the fluorescent material is detached. Therefore, the amount of the amplification product produced can be monitored by measuring the fluorescence intensity, and thereby the amount of the original template cDNA can be estimated. The Molecular Beacon probe is an oligonucleotide having its two terminals modified with a fluorescent material and quenching material, respectively, which probe can be hybridized into the amplification region of a target nucleic acid, and at the same time, can have a hairpin-type secondary structure. The probe does not emit fluorescence while having a hairpin structure, due to the presence of the quenching material, but emits fluorescence during annealing, as the probe is hybridized into the target nucleic acid, and the distance between the fluorescent material and the quenching material is widened. Therefore, the amount of the amplification product produced can be monitored by measuring the fluorescence intensity, and thereby the amount of the original template cDNA can be estimated. Since real time RT-PCR is capable of monitoring the amount of PCR amplification in real time, electrophoresis is unnecessary, and thus the expression of the TRAIL signal activator sensitivity marker genes can be analyzed more rapidly.

The expression levels of the TRAIL signal activator sensitivity marker genes thus measured are preferably compared with each other after correcting the levels of each cancer cell by using a housekeeping gene such as GAPDH, β-globin, PGK1 or the like, as an internal standard.

The expression of the TRAIL signal activator sensitivity marker proteins in a sample collected from a test subject mammal can be examined by preparing a protein fraction from the sample, and detecting the translation products of the marker genes (that is, marker protein) contained in the fraction. The detection of the marker proteins can be performed according to an immunological assay method (for example, ELISA, FIA, RIA, Western blotting, immunohistostaining, etc.), using antibodies to the respective proteins, or in the case of proteins exhibiting a measurable physiological activity, such as an enzyme or the like, the detection can be performed by measuring the physiological activity using known techniques for the respective marker proteins. Or else, the detection of marker proteins can also be performed using a mass analysis method such as MALDI-TOFMS or the like.

Furthermore, the antibody to each of the marker proteins can be obtained according to conventionally used polyclonal antibody or monoclonal antibody production techniques, using as a sensitizing antibody, a protein encoded by a gene containing the same or substantially the same base sequence as the base sequence represented by SEQ ID NO: n (n=1, 3, 5, 7, 9, 11 or 13), or an orthologue of a human gene containing the base sequence represented by the SEQ ID No. in an other mammal, or a partial peptide thereof, and more specifically, a protein containing the same or substantially the same amino acid sequence as the amino acid sequence represented by SEQ ID NO: n (n=2, 4, 6, 8, 10, 12 or 14) or a partial amino acid sequence thereof, or an orthologue of the protein in an other mammal, or a partial peptide thereof. Here, the ‘substantially the same amino acid sequence’ means an amino acid sequence encoded by the above-described ‘substantially the same base sequence’.

In applying the individual immunological assay methods to the examination of the TRAIL signal activator sensitivity marker proteins, it is not necessary to establish particular conditions or carry out particular operations. An appropriate measurement system for the TRAIL signal activator sensitivity marker proteins can be constructed by giving technical considerations that are obvious to those skilled in the art, to the conditions and operations which are conventional in the respective methods. For details of these conventional technical means, reference may be made to review articles and monographs. For example, reference can be made to “Radioimmunoassay,” edited by Hiroshi Irie (Kodansha, published in 1974), “Radioimmunoassay, Supplemental,” edited by Hiroshi Irie (Kodansha, published in 1979), “Enzyme Immunoassay,” edited by Eiji Ishikawa et al. (Igaku Shoin, published in 1978), “Enzyme Immunoassay” (2^{nd} edition), edited by Eiji Ishikawa, et al. (Igaku Shoin, published in 1982), “Enzyme Immunoassay” (3^{rd} edition) edited by Eiji Ishikawa, et al. (Igaku Shoin, published in 1987), “Methods In Enzymology”, Vol. 70 (Immunochemical Techniques (Part A)), ibid., Vol. 73 (Immunochemical Techniques (Part B)), ibid., Vol. 74 (Immunochemical Techniques (Part C)), ibid., Vol. 84 (Immunochemical Techniques (Part D: Selected Immunoassays), ibid., Vol. 92 (Immunochemical Techniques (Part E: Monoclonal Antibodies and General Immunoassay Methods)), ibid., Vol. 121 (Immunochemical Techniques (Part I: Hybridoma Technology and Monoclonal Antibodies)) (published by Academic Press), “Enzyme Antibody Method” edited by Keiichi Watanabe, et al. (Gakusai, published in 1992), and the like.

The activity of the TRAIL signal activator sensitivity marker proteins in a sample collected by a test subject mammal can be measured using methods known per se in accordance with the physiological activity of the respective marker proteins.

For example, the activity of STK17B can be measured by detecting the phosphorylation of a substrate protein or peptide. Since STK17B has a self-phosphorylation activity, when the phosphorylation of STK17B itself is detected, it is not essential to add other substrates to the reaction system. However, if desired, for example, myosin light chain kinase, which is a physiological substrate protein of STK17B, or its fragments containing the target phosphorylation sites, or various known synthetic peptides as the substrates for serine/threonine kinase assay, can also be used. The phosphorylation reaction is conducted by dissolving the sample in an appropriate buffer solution [for example, phosphate buffer solution, Tris-HCl buffer solution (pH about 4 to about 10, preferably pH about 6 to about 8), etc.], further adding a phosphate group donor (for example, ATP, etc.) and if necessary, a substrate protein (peptide), and incubating the system typically at about 20 to about 40° C., preferably at about 30 to about 37° C., for about 30 minutes to several hours. The measurement of phosphorylation can be performed by, for example, conducting a reaction using a labeled phosphate group donor (for example, [γ-³²P]ATP, etc.), and detecting the amount of label in STK17B and/or the other substrate protein (peptide). Alternatively, the reaction solution is contacted with a material which is capable of adsorbing a protein (peptide) (for example, DE81 membrane, cellulose membrane, PVDF membrane, etc.), and the amount of the label bound to the material can be measured. Furthermore, phosphorylation property of the substrate can also be detected using a change in the total charge or a change in the molecular weight as an index. In addition, if an antibody specific to the phosphorylation substrate is available, the phosphorylation of the substrate protein (peptide) can be detected using an antigen-antibody reaction.

For the activity of CASP8, known synthetic peptides can be used as the substrate for a caspase-8 activity assay.

The “fluctuation” in the expression or activity of the TRAIL signal activator sensitivity markers in the sample measured as described above, can be evaluated, for example, through a comparison with reference values (cut-off values) calculated in advance by the following technique.

First, the expression level or activity of each of the TRAIL signal activator sensitivity markers in two or more known TRAIL signal activator sensitive cancer cells and two or more known TRAIL signal activator insensitive cancer cells (learning cancer cells), is measured by the method as described above. The cancer cells used herein are not particularly limited, but they are preferably derived from a mammal of the same species as the test subject. The type of the cancer tissue is not particularly limited, and cells derived from any of the above-described cancers can be used. The sensitivity/insensitivity of cancer cells to a TRAIL signal activator can be determined by culturing the cancer cells in the presence of a TRAIL signal activator (for example, TRAIL, anti-TRAILR1 agonist antibody, anti-TRAILR2 agonist antibody, etc.) at various concentrations, and defining those cancer cells which give a certain growth inhibitory effect, to be sensitive. The certain growth inhibitory effect may be exemplified by an IC50 of 100 nM or less, or the like, but without limitation, a person having ordinary skill in the art can arbitrarily set another adequate standard. As the learning cancer cells, it is preferable to test preferably 5 or more, and more preferably 10 or more of TRAIL signal activator sensitive and insensitive cancer cells.

The “known TRAIL signal activator sensitive cancer cells” refer to cancer cells known to be sensitive to TRAIL signal activator, irrespective of whether the cancer cells per se are known or otherwise.

The “known TRAIL signal activator insensitive cancer cells” refer to cancer cells known to be insensitive to TRAIL signal activator, irrespective of whether the cancer cells per se are known or otherwise.

When whether the cancer cells to be used are sensitive or insensitive to TRAIL signal activator is not known, such can be confirmed by the method of, for example, Example 1 to be mentioned below.

Subsequently, among the learning cancer cells, those arbitrarily selected cancer cells are defined as reference cancer cells. The reference cancer cells may be either sensitive or insensitive to the TRAIL signal activator. Also, the cancer cells may be cancer cells belonging to any tissue. The relative expression level or relative activity in other cancer cells is calculated with respect to the expression level or activity of the respective TRAIL signal activator sensitivity markers in the reference cancer cells.

One of the methods of setting the reference value (cut-off value) for the expression level (activity) of a TRAIL signal activator sensitivity marker (Method 1) according to the invention, is a method of taking a value of the relative expression level or relative activity which enables accurately determining the presence or absence of the sensitivity of the learning cancer cells to the TRAIL signal activator from the relationship between the relative expression level or relative activity of the marker and the sensitivity to TRAIL signal activator in the respective cancer cells, as the reference value for the marker. Here, the term “accurately determining” means that the sensitivity/insensitivity to the TRAIL signal activator in at least 60%, preferably at least 70%, and more preferably at least 80%, of cancer cells among the entire learning cancer cells can be accurately determined. The method may accurately determine both the sensitivity and insensitivity, or the method may accurately determine any one of them.

When the relative expression level or relative activity of a TRAIL signal activator sensitivity marker in a sample collected from a test subject, with respect to the expression level or activity of the TRAIL signal activator sensitivity marker in the known cancer cells, attains a value which falls on the side of being sensitive relative to the reference value determined as described above, the test subject is selected as a TRAIL signal activator sensitive patient. On the other hand, when the test subject attains a value which falls on the side of being insensitive relative to the reference value, the test subject is considered as a TRAIL signal activator insensitive patient, and is excluded from the subject of administration of the TRAIL signal activator. Whether a value equal to or greater than the reference value is sensitive or insensitive, varies depending on the TRAIL signal activator sensitivity marker, but in the case where the test subject is a patient of breast cancer or colon cancer, for AIM1, a value equal to or greater than the reference value is determined to be TRAIL signal activator insensitive. Meanwhile, for STK17B, LOC93349, CASP8, SP110, NOD27 and RHOBTB3, regardless of the type of cancer, when one or more, preferably two or more values are equal to or greater than the reference value, the test subject can be determined to be TRAIL signal activator sensitive, and when one or more, preferably two or more values are less than the reference value, the test subject can be determined to be TRAIL signal activator insensitive.

The reference value of AIM1 is, for example, obtained by comparing the relationship between the relative expression level (two places of decimals) or relative activity (two places of decimals) of AIM1 in the above-mentioned cancer cells and the presence or absence of TRAIL signal activator sensitivity in said cells, selecting the highest relative expression level A (1.93 of colon cancer DLD1 in Tables 2 and 4) or relative activity A in the TRAIL signal activator sensitive cancer cell group, selecting relative expression level B (2.27 of Zr75-1 of breast cancer in Tables 2 and 4) or relative activity B in the TRAIL signal activator insensitive cancer cell group, which is greater than the relative expression level A or relative activity A and the nearest thereto, and eliminating the second place of decimal from the value of relative expression level B or relative activity B (2.2 in Tables 2 and 4).

The reference values of STK17B, LOC93349, CASP8, SP110, NOD27 and RHOBTB3 are obtained by comparing the relationship between the relative expression level (two places of decimals) or relative activity (two places of decimals) of STK17B, LOC93349, CASP8, SP110, NOD27 and RHOBTB3 in the above-mentioned other cancer cells and the presence or absence of TRAIL signal activator sensitivity, and set to 80%-120% value of the median value of the relative expression levels or relative activity in the TRAIL signal activator sensitive cancer cell group, preferably the median value. In a more preferable embodiment, the reference value of STK17B, LOC93349, CASP8, SP110, NOD27 or RHOBTB3 is taken as the median value of each relative expression level (two places of decimals), and when the values of two or more relative expression levels are not less than the reference value, the cell line is ranked as TRAIL signal activator sensitive, and when the values of two or more relative expression levels are less than the reference value, the cell line is ranked as TRAIL signal activator insensitive.

As another method of setting the reference value (cut-off value) for the expression level (activity) of a TRAIL signal activator sensitivity marker (Method 2) according to the invention, there may be mentioned a method of taking a value of the relative expression level or relative activity which enables accurately determining the sensitivity of cancer cells to the TRAIL signal activator from the relationship between the relative expression level or relative activity of the marker in the respective cancer cells and the sensitivity to the TRAIL signal activator, as the upper reference value for the marker, while taking the value of the relative expression level or relative activity which enables accurately determining the insensitivity of cancer cells to the TRAIL signal activator, as the lower reference value for the marker. Here, the term “sensitivity to the TRAIL signal activator is accurately determined” means that the ratio of sensitive cancer cells having a measured value equal to or greater than the upper reference value, with respect to the entire cancer cells, is at least 60%, preferably at least 70%, and more preferably at least 80%. The term “insensitivity to the TRAIL signal activator is accurately determined” means that the ratio of insensitive cancer cells having a measured value less than the lower reference value in the entire cancer cells is at least 60%, preferably at least 70%, and more preferably at least 80%.

If the relative expression level or relative activity of a TRAIL signal activator sensitivity marker in a sample collected from a test subject, with respect to the expression level or activity of the TRAIL signal activator sensitivity marker in the reference cancer cells, is equal to or greater than the upper reference value determined as described above, the test subject is selected as a TRAIL signal activator sensitive patient. On the other hand, if the relative expression level or relative activity is less than the lower reference value, the test subject is considered as a TRAIL signal activator insensitive patient, and can be excluded from the subject administration of the TRAIL signal activator. Also, in the case where the relative expression level or relative activity is equal to or greater than the lower reference value, and less than the upper reference value, it can be said to be indeterminable.

As discussed in the above, in a preferred embodiment of the invention, a TRAIL signal activator sensitive patient is selected on the basis of the measurement values of two or more, more preferably three or more, and even more preferably four or more markers selected from the above-mentioned seven TRAIL signal activator sensitivity markers. In this case, for each of the markers, the reference value (cut-off value) can be set by selecting any of the Method 1 and Method 2 described above. Also, when the same reference values are initially provided for a plurality of markers, a value which enables accurately determining the sensitivity/insensitivity to the TRAIL signal activator in the learning cancer cells may be set as a unified reference value for those markers.

Although there are cases where the determination varies with the markers when using two or more markers, the sensitivity/insensitivity to the TRAIL signal activator can be determined by, for example, scoring the determination for the respective markers, and determining in accordance with the comprehensive scores. For example, with regard to the markers for which a single reference value is set, a method can be employed, in which a point is added if the measured value falls on the side of being sensitive from the reference value, while a point is either not added or subtracted if the measured value falls on the side of being insensitive. On the other hand, with regard to the markers for which an upper reference value and a lower reference value are set, a method can be employed, in which a point is added if the measured value is equal to or greater than the upper reference value; a zero point is given if the measured value is equal to or greater than the lower reference value and less than the upper reference value; and a point is subtracted if the measured value is less than the lower reference value.

Furthermore, the points may also be weighted according to the respective markers. The point of a marker which contributes more to the determination of sensitivity/insensitivity, can be increased compared to the points of other markers.

In a preferred embodiment of the invention, a TRAIL signal activator sensitive patient is selected according to the following criteria of determination, using AIM1, STK17B, LOC93349 and CASP8 as the TRAIL signal activator sensitivity markers.

(i) For AIM1, the reference value (the reference value which enables accurately determining the insensitivity to the TRAIL signal activator) is set according to the Method 1 described above.

(ii) For STK17B, LOC93349 and CASP8, the upper reference value and the lower reference value are set according to the Method 2 described above.

(iii) A test subject corresponding to the following (a) or (b) is screened as a TRAIL signal activator sensitive patient.

(a) If the test subject is a patient suffering from breast cancer or colon cancer, the level of expression or activity of AIM1 in a sample collected from the test subject is less than the reference value, and given that a +1 point is scored if the level of expression or activity of STK17B, LOC93349 or CASP8 in the sample collected from the test subject is equal to or greater than the upper reference value; a zero point is scored if the level of expression or activity is equal to or greater than the lower reference value and also less than the upper reference value; and a −1 point is scored if the level of expression or activity is less than the lower reference value, the sum of the points for STK17B, LOC93349 and CASP8 is a positive value; and

(b) in case the test subject is a patient suffering from a cancer other than breast cancer and colon cancer, given that a +1 point is scored if the level of expression or activity of STK17B, LOC93349 or CASP8 in the sample collected from the test subject is equal to or greater than the upper reference value; a zero point is scored if the level of expression or activity is equal to or greater than the lower reference value and also less than the upper reference value; and a −1 point is scored if the level of expression or activity is less than the lower reference value, the sum of the points for STK17B, LOC93349 and CASP8 is a positive value.

The reference value for AIM1, and the reference values for STIK17B, LOC93349 and CASP8 vary depending on the type of the reference cancer cells selected in regard to the learning cancer cells, the setting of the level of probability of correct inspection in the learning cancer cells and the like.

For example, in a more preferable embodiment of the present invention, TRAIL signal activator sensitive markers are AIM1, STK17B, LOC93349 and CASP8,

(1) the expression level or activity of AIM1, STK17B, LOC93349 and CASP8 in two or more known TRAIL signal activator sensitive cancer cells and two or more known TRAIL signal activator insensitive cancer cells is measured,

(2)(i) using the expression level or activity of AIM1, STK17B, LOC93349 and CASP8 in one cell arbitrarily selected from TRAIL signal activator sensitive cancer cells and TRAIL signal activator insensitive cancer cells (reference cancer cell) as measured in (1) as 1.0, the relative expression level or relative activity of AIM1, STK17B, LOC93349 and CASP8 in other cancer cells is calculated,

(ii) the relationship between the relative expression level (two places of decimals) or relative activity (two places of decimals) of AIM1 in other cells as measured in (1) and the presence or absence of TRAIL signal activator sensitivity in said cells is compared, the highest relative expression level A (1.93 of colon cancer DLD1 in Tables 2 and 4) or relative activity A in the TRAIL signal activator sensitive cancer cell group is selected, relative expression level B (2.27 of breast cancer Zr75-1 in Tables 2 and 4) or relative activity B in the TRAIL signal activator insensitive cancer cell group, which is greater than the relative expression level A or relative activity A and the nearest thereto, and the value of relative expression level B or relative activity B less the second place of decimal (2.2 in Tables 2 and 4) is taken as the reference value,

(iii) the relationship between the relative expression level (two places of decimals) or relative activity (two places of decimals) of STK17B, LOC93349 and CASP8 in other cells as measured in (1) and the presence or absence of TRAIL signal activator sensitivity in said cells is compared, 80% value of the median value of the relative expression level or relative activity in the TRAIL signal activator sensitive cancer cell group is taken as the upper reference value, and 120% value of the median value of the relative expression level or relative activity in the TRAIL signal activator insensitive cancer cell group is taken as the lower reference value,

a test subject corresponding to the following (a) or (b) is screened as a TRAIL signal activator sensitive patient:

(a) in case the test subject is a patient suffering from breast cancer or colon cancer, the expression level or activity of AIM1 in a sample collected from the test subject is less than the reference value, and given that a +1 point is scored if the expression level or activity of STK17B, LOC93349 or CASP8 in the sample collected from the test subject is equal to or greater than the upper reference value; a zero point is scored if the expression level or activity is equal to or greater than the lower reference value and also less than the upper reference value; and a −1 point is scored if the expression level or activity is less than the lower reference value, the sum of the points for STK17B, LOC93349 and CASP8 is a positive value; and

(b) in case the test subject is a patient suffering from a cancer other than breast cancer and colon cancer, given that a +1 point is scored if the expression level or activity of STK17B, LOC93349 or CASP8 in the sample collected from the test subject is equal to or greater than the upper reference value; a zero point is scored if the expression level or activity is equal to or greater than the lower reference value and also less than the upper reference value; and a −1 point is scored if the expression level or activity is less than the lower reference value, the sum of the points for STK17B, LOC93349 and CASP8 is a positive value.

To be specific, as shown in EXAMPLE 4(2) below, the expression levels of AIM1, STK17B, LOC93349 and CASP8 are the expression levels of mRNAs encoding them, the relative expression level is the relative value to the expression level in COLO205 cell line, the reference value for AIM1 is 2.2, the upper reference value is 1.036 and the lower reference value is 0.514 for STK17B, the upper reference value is 0.666 and the lower reference value is 0.211 for LOC93349, and the upper reference value is 0.833 and the lower reference value is 0.519 for CASP8.

In addition, the question of to what extent the rate of correct diagnosis would be set in the learning cancer cells, or the like. However, for example, when the reference cells are selected to be COLO205 cell line (Cancer Res., 38:1345 (1978); commercially available from Dainippon Sumitomo Pharma Co., Ltd.), which is a human colon adenocarcinoma cell line, as will be described in Example 4(1) below, and the expression of the TRAIL signal activator sensitivity markers is evaluated using the level of expression of mRNA as an index, the reference value for AIM1 is set to 2.2, while the upper reference value and the lower reference value for STK17B, LOC93349 and CASP8 are set to 0.7 and 0.5, respectively, whereby a TRAIL signal activator sensitive patient can also be selected with extremely high accuracy.

As discussed in the above, the TRAIL signal activator contained in the preventive or remedy agent of the invention is preferably TRAIL, an agonist compound of TRAILR1 or TRAILR2, or the like.

TRAIL used in the present invention is the protein comprising the same or substantially the same amino acid sequence as the amino acid sequence represented by SEQ ID NO: 16. TRAIL of the present invention may be a naturally occurring TRAIL protein derived from cells [for example, hepatocytes, splenocytes, nerve cells, glial cells, β cells of pancreas, bone marrow cells, mesangial cells, Langerhans' cells, epidermic cells, epithelial cells, goblet cells, endothelial cells, smooth muscle cells, fibroblasts, fibrocytes, myocytes, fat cells, immune cells (e.g., macrophages, T cells, B cells, natural killer cells, mast cells, neutrophils, basophils, eosinophils, monocytes), megakaryocytes, synovial cells, chondrocytes, bone cells, osteoblasts, osteoclasts, mammary cells, or interstitial cells; or the corresponding precursor cells thereof, stem cells, cancer cells, etc.] of human and other warm-blooded animals (e.g., simian, bovine, horse, swine, sheep, goat, rabbit, mouse, rat, guinea pig, hamster, fowl, etc.); or any tissue or organ where such cells are present [for example, brain or each part of brain (e.g., olfactory bulb, amygdaloid nucleus, basal ganglia, hippocampus, thalamus, hypothalamus, cerebral cortex, medulla oblongata, cerebellum), spinal cord, hypophysis, stomach, pancreas, kidney, liver, gonad, thyroid, gall-bladder, bone marrow, adrenal gland, skin, muscle, lung, gastrointestinal tract (e.g., large intestine and small intestine), blood vessel, heart, thymus, spleen, submandibular gland, peripheral blood, prostate, testis, ovary, placenta, uterus, bone, joint, adipose tissue (e.g., brown adipose tissue, white adipose tissue), skeletal muscle, etc.]. TRAIL may also be a protein synthesized chemically, or biochemically by a cell-free protein synthesis system, as described hereinbelow. Alternatively, TRAIL of the present invention may be a recombinant protein produced from a transfectant in which the acid comprising a base sequence encoding the above-mentioned amino acid sequence is transfected.

The amino acid sequence comprising substantially the same amino acid sequence as that represented by SEQ ID NO: 16 includes amino acid sequences having at least about 50% homology, preferably at least about 60% homology, more preferably at least about 70% homology, even more preferably at least about 80% homology, particularly preferably at least about 90% homology and most preferably at least about 95% homology, to the amino acid sequence shown by SEQ ID NO: 16. Herein, the ‘Homology’ means a ratio (%) of the same amino acid and similar amino acid residue to the total overlapped amino acid residue, in the best alignment when two amino acid sequences are aligned with the use of a mathematical algorithm commonly known in the technical field (preferably, the algorithm is obtained by consider allowing gaps on one or both side of the sequence for the best alignment). The term ‘similar amino acid’ refers to an amino acid similar in its physiochemical properties, and the examples include amino acids classified in a same group such as aromatic amino acid (Phe, Trp, Tyr), alifhatic amino acid (Ala, Leu, Ile, Val), polar amino acid (Gln, Asn), basic amino acid (Lys, Arg, H is), acidic amino acid (Glu, Asp), amino acid including a hydroxyl group (Ser, Thr), amino acid having a short side chain (Gly, Ala, Ser, Thr, Met), and the like. A substitution by such similar amino acid is expected to give no change in the phenotype of protein (thus is a conservative amino acid substitution). A specific example of the conservative amino acid substitution is well-known in the technical field, and is disclosed in various documents (for example, refer Bowie et al, Science, 247: 1306-1310 (1990)).

Homology of the amino acid sequences in the present specification can be measured under the following conditions (an expectation value=10; gaps are allowed; matrix=BLOSUM62; filtering=OFF) using a homology scoring algorithm NCBI BLAST (National Center for Biotechnology Information Basic Local Alignment Search Tool). Other algorithms for determining homology of the amino acid sequence may be also exemplified by the above mentioned homology scoring algorithms of the base sequences.

The protein comprising substantially the same amino acid sequence as the amino acid sequence represented by SEQ ID NO: 16 is such protein comprising substantially the same amino acid sequence as the amino acid sequence represented by SEQ ID NO: 16, and having an activity substantially equivalent to the protein comprising the amino acid sequence represented by SEQ ID NO: 16. As the substantially equivalent activity described above, there are, for example, the binding activity to TRAILR1 and TRAILR2, and TRAIL signal transduction activity [for example, activity in activating caspase-8, activity in activating execution type caspase (caspase-3, -6, -7, -9, and the like), apoptosis induce activity, and the like]. The substantially equivalent is used to mean that the nature of these properties is equivalent in terms of quality (e.g., physiologically or pharmacologically). Thus, the activities described above are preferably equivalent (e.g., about 0.01 to 100 times, preferably about 0.1 to 10 times, more preferably 0.5 to 2 times), but differences in quantitative factors such as a level of these activities may be present and allowable. TRAIL activity can be assayed by publicly known methods.

Examples of TRAIL used in the present invention include proteins such as proteins having (1) the amino acid sequence represented by SEQ ID NO: 16, of which at least 1 or 2 [e.g., preferably about 1 to about 30, more preferably about 1 to about 10 and most preferably 1 to several (1 to 5)] amino acids are deleted; (2) the amino acid sequence represented by SEQ ID NO: 16, to which at least 1 or 2 [e.g., preferably about 1 to about 30, more preferably about 1 to about 10 and most preferably 1 to several (1 to 5)] amino acids are added; (3) the amino acid sequence represented by SEQ ID NO: 16, in which at least 1 or 2 [e.g., preferably about 1 to about 30, more preferably about 1 to about 10 and most preferably 1 to several (1 to 5)] amino acids are inserted; (4) the amino acid sequence represented by SEQ ID NO: 16, in which at least 1 or 2 [e.g., preferably about 1 to about 30, more preferably about 1 to about 10 and most preferably 1 to several (1 to 5)] amino acids are substituted by other amino acids; or (5) a combination of these amino acid sequences; and further include a protein comprising substantially the same amino acid sequence as the amino acid sequence represented by SEQ ID NO: 16.

Where the amino acid sequence is inserted, deleted or substituted as described above, the position of its insertion, deletion, or substitution is not particularly limited, as long as it does not deteriorate the activity of the protein.

In the present specification, the proteins specified by the amino acid sequence are represented in accordance with the conventional way of describing proteins, that is, the N-terminus (amino terminus) at the left hand and the C-terminus (carboxyl terminus) at the right hand. In TRAIL used in the present invention which includes the protein comprising the amino acid sequence represented by SEQ ID NO: 16, the C-terminus may be in any form of a carboxyl group (—COOH), carboxylate (—COO⁻), an amide (—CONH₂) and an ester (—COOR).

Herein, examples of the ester group shown by R include a C₁₋₆ alkyl group such as methyl, ethyl, n-propyl, isopropyl, n-butyl, etc.; a C₃₋₈ cycloalkyl group such as cyclopentyl, cyclohexyl, etc.; a C₆₋₁₂ aryl group such as phenyl, α-naphthyl, etc.; a C₇₋₁₄ aralkyl such as a phenyl-C₁₋₂ alkyl group, e.g., benzyl, phenethyl, etc.; an α-naphthyl-C₁₋₂ alkyl group such as α-naphthylmethyl, etc.; pivaloyloxymethyl and the like.

Where the protein used in the present invention contains a carboxyl group (or a carboxylate) at a position other than the C-terminus, the carboxyl group may be amidated or esterified and such an amide or ester is also included within the protein used in the present invention. Examples of the ester group in this case may be the C-terminal esters described above, etc.

Furthermore, examples of the protein used in the present invention include variants wherein the amino group at the N-terminal amino acid residues (e.g., methionine residue) is protected with a protecting group (e.g., a C₁₋₆ acyl group such as a C₁₋₆ alkanoyl group, e.g., formyl group, acetyl group, etc.); those wherein the N-terminal region is cleaved in vivo and the glutamyl group thus formed is pyroglutaminated; those wherein substituents (e.g., —OH, —SH, amino group, imidazole group, indole group, guanidino group, etc.) on the side chains of amino acids in the molecule are protected with suitable protecting groups (e.g., a C₁₋₆ acyl group such as a C₁₋₆ alkanoyl group, e.g., formyl group, acetyl group, etc.), or conjugated proteins such as glycoproteins having sugar chains; etc.

Specific examples of the protein used in the invention include human TRAIL comprising the amino acid sequence represented by SEQ ID NO: 16, ortholog thereof in other warm-blooded animals, and the like. The amino sequence of ortholog of other warm-blooded animals may be available by the same method as described for ortholog of TRAIL signal activator sensitivity marker genes.

The partial peptide of TRAIL used in the present invention may be any peptide as long as it is a partial peptide comprising the same or substantially the same partial amino acid sequence as the amino acid sequence represented by SEQ ID NO: 16, and having an activity substantially equivalent to the TRAIL used in the invention. Herein, the ‘activity substantially equivalent’ means the same as mentioned above. The ‘activity substantially equivalent’ can also be assayed in a same manner as mentioned above.

For example, there are specifically used peptides containing, e.g., at least 50, preferably at least 70, more preferably at least 100 amino acid sequence, in the constituent amino acid sequence of TRAIL used in the present invention, etc. The partial peptide used in the present invention may be peptides containing (1) the amino acid sequence, of which at least 1 or 2 [preferably about 1 to about 30, more preferably about 1 to about 10 and most preferably 1 to several (1 to 5)] amino acids may be deleted; (2) the amino acid sequence, to which at least 1 or 2 [preferably about 1 to about 30, more preferably about 1 to about 10 and most preferably 1 to several (1 to 5)] amino acids may be added; (3) the amino acid sequence, in which at least 1 or 2 [preferably about 1 to about 30, more preferably about 1 to about 10 and most preferably 1 to several (1 to 5)] amino acids may be inserted; or (4) the amino acid sequence, in which at least 1 or 2 [preferably about 1 to about 10, more preferably 1 to several (1 to 5)] amino acids may be substituted by other amino acids; or (5) a combination of these amino acid sequences.

In the partial peptide of TRAIL used in the present invention, the C-terminus may be in any form of a carboxyl group (—COOH), a carboxylate (—COO⁻), an amide (—CONH₂) or an ester (—COOR). Herein, as the ‘R’ in ester, same ones as in the TRAIL used in the present invention can be exemplified. Where the partial peptide contains a carboxyl group (or a carboxylate) at a position other than the C-terminus, the carboxyl group may be amidated or esterified and such an amide or ester is also included within the partial peptide of TRAIL used in the present invention. Examples of the ester group in this case may be the C-terminal esters described above, etc. Furthermore, the partial peptide includes those wherein the amino group at the N-terminal amino acid residues (e.g., methionine residue) is protected with a protecting group; those wherein the N-terminal region is cleaved in vivo and the glutamyl group thus formed is pyroglutaminated; those wherein a substituent on the side chain of an amino acid in the molecule is protected with a suitable protecting group, or conjugated peptides such as so-called glycopeptides having sugar chains; etc., as in the TRAIL described above.

As TRAIL, its partial peptide, or salts thereof used in the present invention, salts with physiologically acceptable acids (e.g., inorganic acids, organic acids, etc.) or bases (e.g., alkali metals, etc.), preferably physiologically acceptable acid addition salts can be used. Examples of such salts include salts with inorganic acids (e.g., hydrochloric acid, phosphoric acid, hydrobromic acid, sulfuric acid), salts with organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid) and the like.

TRAIL or salts thereof used in the present invention may be prepared by publicly known methods used to purify a protein from human or warm-blooded animal cells or tissues described above. Specifically, human or non-human mammalian tissues or cells are homogenized, extracted with acid or the like, and then the extract is treated by a combination of chromatography techniques such as reverse phase chromatography, ion exchange chromatography, and the like, thereby TRAIL or salts thereof used in the present invention can be prepared.

TRAIL, its partial peptide, or salts thereof used in the present invention (hereinafter may be inclusively abbreviated to ‘TRAILs’) can be manufactured by publicly known methods for peptide synthesis.

For the methods for peptide synthesis, for example, either solid phase synthesis or liquid phase synthesis may be used. The object protein can be produced by condensing partial peptide or amino acids that can construct the protein of the invention with the remaining part and, when the resulting product contains a protecting group, removing the protecting group. Here, the condensation and elimination of the protecting groups may be carried out in accordance with publicly known methods, for examples, the methods described in (1) and (2) below.

(1) M. Bodanszky and M. A. Ondetti: Peptide Synthesis, Interscience Publishers, New York (1966)

(2) Schroeder & Luebke: The Peptide, Academic Press, New York (1965)

To synthesize TRAILs of the invention, commercially available resins that are used for protein synthesis may be used. Examples of such resins include chloromethyl resin, hydroxymethyl resin, benzhydrylamine resin, aminomethyl resin, 4-benzyloxybenzyl alcohol resin, 4-methylbenzhydrylamine resin, PAM resin, 4-hydroxymethylmethylphenyl acetamidomethyl resin, polyacrylamide resin, 4-(2′,4′-dimethoxyphenyl-hydroxymethyl)phenoxy resin, 4-(2′,4′-dimethoxyphenyl-Fmoc-aminoethyl) phenoxy resin, etc. Using these resins, amino acids, in which α-amino groups and functional groups on the side chains are appropriately protected, are condensed on the resin in accordance with the sequence of the objective protein according to various condensation methods publicly known in the art. At the end of the reaction, the protein or partial peptide is excised from the resin and at the same time, the protecting groups are removed. Then, intramolecular disulfide bond-forming reaction is performed in a highly diluted solution to obtain the objective protein or partial peptide, or amides thereof.

For condensation of the protected amino acids described above, a variety of activation reagents for protein synthesis may be used, and carbodiimides are particularly employed. Examples of such carbodiimides include DCC, N,N′-diisopropylcarbodiimide, N-ethyl-N′-(3-dimethylaminopropyl)carbodiimide, etc. For activation by these reagents, the protected amino acids in combination with a racemization inhibitor (e.g., HOBt, HOOBt) are added directly to the resin, or the protected amino acids are previously activated in the form of anhydrides of the objective acids, as HOBt esters or HOOBt esters, followed by adding the thus activated protected amino acids to the resin.

Solvents suitable for use to activate the protected amino acids or condense with the resin may be appropriately chosen from solvents that are known to be usable for protein condensation reactions. Examples of such solvents are acid amides such as N,N-dimethylformamide, N,N-dimethylacetamide, N-methylpyrrolidone, etc.; halogenated hydrocarbons such as methylene chloride, chloroform, etc.; alcohols such as trifluoroethanol, etc.; sulfoxides such as dimethylsulfoxide, etc.; ethers such as pyridine, dioxane, tetrahydrofuran, etc.; nitriles such as acetonitrile, propionitrile, etc.; esters such as methyl acetate, ethyl acetate, etc.; and appropriate mixtures of these solvents. The reaction temperature is appropriately chosen from the range known to be applicable to protein binding reactions and is usually selected in the range of approximately −20° C. to 50° C. The activated amino acid derivatives are used generally in an excess of 1.5 to 4 times. The condensation is examined using the ninhydrin reaction; when the condensation is insufficient, the condensation can be completed by repeating the condensation reaction without removal of the protecting groups. When the condensation is yet insufficient even after repeating the reaction, unreacted amino acids are acetylated with acetic anhydride or acetylimidazole to avoid any possible effect on the subsequent reaction.

Protection of the functional groups that should not be involved in the reaction of the starting materials, protecting groups, elimination of the protecting groups, and activation of the functional groups involved in the reaction may be appropriately chosen from publicly known groups and publicly known means.

Examples of the protecting groups used to protect the starting amino groups include Z, Boc, t-pentyloxycarbonyl, isobornyloxycarbonyl, 4-methoxybenzyloxycarbonyl, Cl-Z, Br-Z, adamantyloxycarbonyl, trifluoroacetyl, phthaloyl, formyl, 2-nitrophenylsulphenyl, diphenylphosphinothioyl, Fmoc, etc.

A carboxyl group can be protected by, e.g., alkyl esterification (linear, branched or cyclic alkyl esterification of, e.g., methyl, ethyl, propyl, butyl, t-butyl, cyclopentyl, cyclohexyl, cycloheptyl, cyclooctyl, 2-adamantyl, etc.), aralkyl esterification (e.g., benzyl ester, 4-nitrobenzyl ester, 4-methoxybenzyl ester, 4-chlorobenzyl ester, benzhydryl ester, etc.), phenacyl esterification, benzyloxycarbonyl hydrazidation, t-butoxycarbonyl hydrazidation, trityl hydrazidation, or the like.

The hydroxyl group of serine can be protected through, for example, its esterification or etherification. Examples of groups appropriately used for the esterification include a lower (C₁₋₆) alkanoyl group, such as acetyl group, an aroyl group such as benzoyl group, and a group derived from carbonic acid such as benzyloxycarbonyl group, ethoxycarbonyl group, etc. Examples of a group appropriately used for the etherification include benzyl group, tetrahydropyranyl group, t-butyl group, etc.

Examples of groups for protecting the phenolic hydroxyl group of tyrosine include Bzl, Cl₂-Bzl, 2-nitrobenzyl, Br-Z, t-butyl, etc.

Examples of groups used to protect the imidazole moiety of histidine include Tos, 4-methoxy-2,3,6-trimethylbenzenesulfonyl, DNP, benzyloxymethyl, Bum, Boc, Trt, Fmoc, etc.

To eliminate (split off) the protecting groups, there are used catalytic reduction under hydrogen gas flow in the presence of a catalyst such as Pd-black or Pd-carbon; an acid treatment with anhydrous hydrogen fluoride, methanesulfonic acid, trifluoromethanesulfonic acid, trifluoroacetic acid, or a mixture solution of these acids; a treatment with a base such as diisopropylethylamine, triethylamine, piperidine or piperazine; reduction with sodium in liquid ammonia, etc. The elimination of the protecting group by the acid treatment described above is carried out generally at a temperature of approximately −20° C. to 40° C. In the acid treatment, it is efficient to add a cation scavenger such as anisole, phenol, thioanisole, m-cresol, p-cresol, dimethylsulfide, 1,4-butanedithiol, 1,2-ethanedithiol, etc. Furthermore, 2,4-dinitrophenyl group known as the protecting group for the imidazole of histidine is removed by a treatment with thiophenol. Formyl group used as the protecting group of the indole of tryptophan is eliminated by the aforesaid acid treatment in the presence of 1,2-ethanedithiol, 1,4-butanedithiol, etc. as well as by a treatment with an alkali such as a dilute sodium hydroxide solution, dilute ammonia, etc.

Examples of the activated carboxyl groups in the starting material include the corresponding acid anhydrides, azides, activated esters [esters with alcohols (e.g., pentachlorophenol, 2,4,5-trichlorophenol, 2,4-dinitrophenol, cyanomethyl alcohol, p-nitrophenol, HONB, N-hydroxysuccimide, N-hydroxyphthalimide, HOBt)]. As the amino acids in which the amino groups are activated in the starting material, the corresponding phosphoric amides are employed.

In another method for obtaining the amides of the desired protein or partial peptide, for example, the α-carboxyl group of the carboxy terminal amino acid is first protected by amidation; the peptide (protein) chain is then extended from the amino group side to a desired length. Subsequently, a protein or partial peptide, in which only the protecting group of the N-terminal α-amino group of the peptide chain has been eliminated, and a protein or partial peptide, in which only the protecting group of the C-terminal carboxyl group has been eliminated, are made. The two proteins or peptides are condensed in a mixture of the solvents described above. The details of the condensation reaction are the same as described above. After the protected protein or peptide obtained by the condensation is purified, all the protecting groups are eliminated by the method described above to give the desired crude protein or peptide. This crude protein or peptide is purified by various known purification means. Lyophilization of the major fraction gives the amide of the desired protein or peptide.

To prepare the esterified protein or peptide, for example, the α-carboxyl group of the carboxy terminal amino acid is condensed with a desired alcohol to prepare the amino acid ester, which is followed by procedures similar to the preparation of the amidated protein or peptide above to give the desired esterified protein or peptide.

The partial peptide of TRAIL or a salt thereof used in the present invention can be manufactured by cleaving TRAIL or a salt thereof obtained by any method of the above described or to be described later, with an appropriate peptidase.

The TRAILs used in the invention obtained in the above manner may be purified and isolated by conventional purification methods such as solvent extraction, distillation, column chromatography, liquid chromatography and recrystallization, and a combination of these.

When the protein or partial peptide obtained by the above methods is in a free form, the free form can be converted into an appropriate salt by a publicly known method or its modification; conversely when the protein is obtained in a salt form, it can be converted into a free form or other different salt form by a publicly known method or its modification.

TRAILs in the invention can also be manufactured by culturing a transformant to which an expression vector containing polynucleotide encoding TRAILs or its partial peptide is introduced to give TRAILs, and then by separating and purifying TRAILs from the obtained culture.

The nucleic acid encoding TRAILs or its partial peptide may be any nucleic acids, as long as it contains base sequence encoding an amino acid sequence of TRAIL or its partial amino acid sequence. The polynucleotide may be DNA or RNA, or DNA/RNA chimera, and preferably is DNA. In addition, the polynucleotide may be a double-strand, or single-strand. The double-strand may include a double-stranded DNA, a double-stranded RNA, and DNA:RNA hybrid.

DNA encoding TRAILs or its partial peptide can be exemplified by genomic DNA and cDNA derived from human and other warm-blooded animals (e.g., simian, bovine, horse, swine, sheep, goat, rabbit, mouse, rat, guinea swine, hamster, feline, fowl, etc.) cells [for example, hepatocytes, splenocytes, nerve cells, glial cells, β cells of pancreas, bone marrow cells, mesangial cells, Langerhans' cells, epidermic cells, epithelial cells, goblet cells, endothelial cells, smooth muscle cells, fibroblasts, fibrocytes, myocytes, fat cells, immune cells (e.g., macrophages, T cells, B cells, natural killer cells, mast cells, neutrophils, basophils, eosinophils, monocytes), megakaryocytes, synovial cells, chondrocytes, bone cells, osteoblasts, osteoclasts, mammary cells, or interstitial cells; or the corresponding precursor cells, stem cells, cancer cells, etc.]; or any tissues or organs where such cells are present [for example, brain or each part of brain (e.g., olfactory bulb, amygdaloid nucleus, basal ganglia, hippocampus, thalamus, hypothalamus, cerebral cortex, medulla oblongata, cerebellum), spinal cord, hypophysis, stomach, pancreas, kidney, liver, gonad, thyroid, gall-bladder, bone marrow, adrenal gland, skin, muscle, lung, gastrointestinal tract (e.g., large intestine and small intestine), blood vessel, heart, thymus, spleen, submandibular gland, peripheral blood, prostate, testis, ovary, placenta, uterus, bone, joint, adipose tissue (e.g., brown adipose tissue, white adipose tissue), skeletal muscle, etc.] or synthetic DNA. The genomic DNA and cDNA encoding TRAILs or its partial peptide can be directly amplified by Polymerase Chain Reaction (hereinafter, abbreviate to “PCR method”) and Reverse Transcriptase-PCR (hereinafter abbreviate as “RT-PCR method”) with the use of each genomic DNA fraction, and total RNA or mRNA fraction prepared from the above-described cells or tissues as a template. Further, the genomic DNA and cDNA encoding TRAILs or its partial peptide can be respectively cloned from genomic DNA library and cDNA library which are prepared by inserting the fragment of genomic DNA, and total RNA or mRNA prepared from the above-described cells or tissues into an appropriate vector, in accordance with a colony or plaque hybridization assay or PCR method. The vector used for the library may be any of bacteriophage, plasmid, cosmid, phagemid and the like.

Examples of the DNA encoding TRAIL may be any of DNA comprising the base sequence represented by SEQ ID NO: 15, and DNA comprising a base sequence hybridizable to the base sequence represented by SEQ ID NO: 15 under stringent conditions and encoding a protein or peptide which has the activity substantially equivalent to the protein comprising the amino acid sequence represented by SEQ ID NO: 16.

As the DNA that is hybridizable to the base sequence represented by SEQ ID NO: 15 under stringent conditions, there are employed, for example, DNAs comprising base sequences having at least about 60% homology, preferably at least about 70% homology, more preferably at least about 80% homology, and particularly preferably at least about 90% homology, to the base sequence represented by SEQ ID NO: 15.

Homology of the base sequences in the present specification, for example, can be measured under the following conditions (an expectation value=10; gaps are allowed; filtering=ON; match score=1; mismatch score=−3) using a homology scoring algorithm NCBI BLAST (National Center for Biotechnology Information Basic Local Alignment Search Tool).

The hybridization can be carried out by publicly known methods or by modifications thereof, for example, by the method described in Molecular Cloning, 2nd ed. (J. Sambrook et al., Cold Spring Harbor Lab. Press, 1989). A commercially available library can also be used according to the instructions of the attached manufacturer's protocol. The hybridization can be carried out preferably under stringent conditions.

The stringent conditions used herein are, for example, those in a sodium concentration at about 19 to 40 mM, preferably about 19 to 20 mM at a temperature of about 50 to 70° C., preferably about 60 to 65° C., (in particular, hybridization conditions in a sodium concentration at about 19 mM at a temperature of about 65° C. are most preferred). Those skilled in the art can simply regulate to a desired stringency by appropriately changing a salt concentration of hybridization solution, temperature of hybridization reaction, probe concentration, length of probe, number of mismatch, time for hybridization reaction, salt concentration of washing solution, temperature for washing, etc.

Preferable examples of the DNA encoding TRAIL include human TRAIL cDNA comprising the base sequence represented by SEQ ID NO: 15, its allelic variant, or ortholog thereof (for example, mouse, rat, guinea pig, hamster, rabbit, sheep, goat, swine, bovine, horse, bird, feline, canine, simian, chimpanzee, etc.) in other warm-blooded animals, and the like.

DNA encoding the partial peptide of TRAIL may be any DNA so long as it contains the base sequence encoding the same or substantially the same amino acid sequence as a part of the amino acid sequence represented by SEQ ID NO: 16. DNA may also be any of genomic DNA, cDNA derived from the cells and tissues described above, and synthetic DNA.

As the DNA encoding the partial peptide, there is specifically employed, for example, DNA containing (1) a partial base sequence of DNA comprising the base sequence represented by SEQ ID NO: 15; or (2) a base sequence hybridizable to the base sequence represented by SEQ ID NO: 15, under high stringent conditions and also encoding a peptide which has the activity substantially equivalent to the protein containing an amino acid sequence which is encoded by DNA.

As the DNA that is hybridizable to DNA comprising the base sequence represented by SEQ ID NO: 15 under stringent conditions, there are employed, for example, DNAs comprising base sequences having at least about 60% homology, preferably at least about 70% homology, more preferably at least about 80% homology, and particularly preferably at least about 90% homology, to the corresponding part in the base sequence.

For cloning of DNAs that encode TRAIL or its partial peptide, the DNA can be either amplified by PCR using synthetic DNA primers containing a part of the base sequence encoding the protein or peptide, or the DNA inserted into an appropriate expression vector can be hybridized with a labeled DNA fragment or synthetic DNA that encodes a part or entire region of TRAIAL. The hybridization can be carried out, for example, according to the method described in Molecular Cloning, 2nd (described above). Where the hybridization is carried out using commercially available library, the procedures may be conducted in accordance with the protocol described in the instructions attached to the library.

Substitution of the base sequence of DNA can be effected by publicly known methods such as the ODA-LAPCR method, the Gapped duplex method, the Kunkel method, etc., or its modification, using a publicly known kit available as Mutan™-super Express Km (Takara Bio) or Mutan™-K (Takara Bio), etc.

The cloned DNA can be used as it is, depending upon purpose or, if desired, after digestion with a restriction enzyme or after addition of a linker thereto. The DNA may contain ATG as a translation initiation codon at the 5′ end thereof and TAA, TGA or TAG as a translation termination codon at the 3′ end thereof. These translation initiation and termination codons may also be added by using an appropriate synthetic DNA adapter.

The protein or peptide can be manufactured by transforming a host with the expression vector containing the DNA encoding TRAIL or its partial peptide, thereafter culturing the obtained transformant.

The expression vector containing the DNA encoding TRAIL or its partial peptide can be manufactured, for example, by excising the desired DNA fragment from the DNA encoding TRAIL, and then ligating the DNA fragment with an appropriate expression vector downstream a promoter in the vector.

Examples of the expression vector include plasmids derived form E. coli (e.g., pBR322, pBR325, pUC12, pUC13), plasmids derived from Bacillus subtilis (e.g., pUB110, pTP5, pC194), plasmids derived from yeast (e.g., pSH19, pSH15), bacteriophages such as λ phage, etc., animal viruses such as retrovirus, vaccinia virus, baculovirus, etc. as well as pA1-11, pXT1, pRc/CMV, pRc/RSV, pcDNA I/Neo, etc.

The promoter may be any promoter if it matches well with a host to be used for gene expression.

For example, when animal cells are used as the host, examples of the promoter are SRα promoter, SV40 promoter, LTR promoter, CMV (cytomegalovirus) promoter, HSV-tK promoter, etc. Among them, it is preferable to use CMV promoter, SRα promoter, etc.

When bacteria of the genus Escherichia is used as a host, preferred examples of the promoter are trp promoter, lac promoter, recA promoter, λP_{L} promoter, lpp promoter, T7 promoter, etc.

When bacteria of the genus Bacillus is used as the host, preferred example of the promoter are SPO1 promoter, SPO2 promoter, penP promoter, etc.

When yeast is used as the host, preferred examples of the promoter are PHO5 promoter, PGK promoter, GAP promoter, ADH promoter, etc.

When insect cells are used as the host, preferred examples of the promoter are polyhedrin promoter, P10 promoter, etc.

In addition to the foregoing examples, the expression vector may further optionally contain an enhancer, a splicing signal, a poly A addition signal, a selection marker, SV40 replication origin (hereinafter sometimes abbreviated as SV40ori), etc. Examples of the selection marker include dihydrofolate reductase (hereinafter sometimes abbreviated as dhfr) gene [methotrexate (MTX) resistance], ampicillin resistant gene (hereinafter sometimes abbreviated as Amp^{r}), neomycin resistant gene (hereinafter sometimes abbreviated as Neo^{r}, G418 resistance), etc. In particular, when dhfr gene is used as the selection marker using dhfr gene-deficient Chinese hamster cells, selection can also be made on a thymidine free medium.

If necessary, a base sequence encoding a signal sequence (signal codon) that matches with a host may be added to the 5′ end of DNA encoding TRAIL or its partial peptide. The signal sequence that can be used are PhoA signal sequence, OmpA signal sequence, etc. when bacteria of the genus Escherichia is used as the host; α-amylase signal sequence, subtilisin signal sequence, etc. when bacteria of the genus Bacillus is used as the host; MFα signal sequence, SUC2 signal sequence, etc. when yeast is used as the host; and insulin signal sequence, α-interferon signal sequence, antibody molecule signal sequence, etc. when animal cells are used as the host, respectively.

Examples of the host, which may be employed, are bacteria belonging to the genus Escherichia, bacteria belonging to the genus Bacillus, yeast, insect cells, insects, animal cells, etc.

Examples of the bacteria belonging to the genus Escherichia include Escherichia coli K12 DH1 [Proc. Natl. Acad. Sci. USA, 60: 160 (1968)], JM103 [Nucleic Acids Res., 9: 309 (1981)], JA221 [J. Mol. Biol., 120: 517 (1978)], HB101 [J. Mol. Biol., 41: 459 (1969)], C600 [Genetics, 39: 440 (1954)], etc.

Examples of the bacteria belonging to the genus Bacillus include Bacillus subtilis MI114 [Gene, 24: 255 (1983)], 207-21 [J. Biochem., 95: 87 (1984)], etc.

Examples of yeast include Saccharomyces cereviseae AH22, AH22R⁻, NA87-11A, DKD-5D, 20B-12; Schizosaccharomyces pombe NCYC1913, NCYC2036; Pichia pastoris KM71, etc.

Examples of insect cells include, for the virus AcNPV, Spodoptera frugiperda cell (Sf cell), MG1 cell derived from mid-intestine of Trichoplusia ni, High Five™ cell derived from egg of Trichoplusia ni, cells derived from Mamestra brassicae, cells derived from Estigmena acrea, etc.; and for the virus BmNPV, Bombyx mori N cell (BmN cell), etc. is used. Examples of the Sf cell which can be used are Sf9 cell (ATCC CRL1711), Sf21 cell (both cells are described in Vaughn, J. L. et al., In Vivo, 13: 213-217 (1977)), etc.

As the insect, for example, a larva of Bombyx mori can be used [Maeda et al., Nature, 315, 592 (1985)].

Examples of animal cells include COS-7 and Vero cell derived from simian; CHO and dhfr gene-deficient CHO cell (CHO (dhfr⁻)) cell derived from Chinese hamster; L, AtT-20 and myeloma cell derived from mouse; GH3 cell derived from rat; FL, HEK293, HepG2, and HeLa cell derived from human, etc.

Transformation can be carried out, depending on type of the host, in accordance with publicly known methods.

Bacteria belonging to the genus Escherichia can be transformed, for example, by the method described in Proc. Natl. Acad. Sci. USA, 69: 2110 (1972), Gene, 17: 107 (1982), etc.

Bacteria belonging to the genus Bacillus can be transformed, for example, by the method described in Mol. Gen. Genet., 168: 111 (1979), etc.

Yeast can be transformed, for example, by the method described in Meth. Enzymol., 194: 182-187 (1991), Proc. Natl. Acad. Sci. USA, 75: 1929 (1978), etc.

Insect cells or insects can be transformed, for example, according to the method described in Biotechnology (N.Y.), 6: 47-55 (1988), etc.

Animal cells can be transformed, for example, according to the method described in Saibo Kogaku (Cell Engineering), extra issue 8, Shin Saibo Kogaku Jikken Protocol (New Cell Engineering Experimental Protocol), 263-267 (1995) (published by Shujunsha), Virology, 52: 456 (1973).

Culturing of the transformant can be carried out, depending on type of the host, in accordance with publicly known methods.

A preferred example of a medium to be used in culturing is a liquid medium, in the case of culturing the transformant of which the host is bacteria belonging to the genus Escherichia or the genus Bacillus. In addition, the medium preferably contains materials required for growth of the transformant such as carbon sources, nitrogen sources, inorganic materials, and the like. Examples of the carbon sources include glucose, dextrin, soluble starch, sucrose, etc.; examples of the nitrogen sources include inorganic or organic materials such as ammonium salts, nitrate salts, corn steep liquor, peptone, casein, meat extract, soybean cake, potato extract, etc.; and, examples of the inorganic materials are calcium chloride, sodium dihydrogenphosphate, magnesium chloride, etc. In addition, yeast extracts, vitamins, growth promoting factors etc. may also be added to the medium. Preferably, pH of the medium is adjusted to about 5 to about 8.

A preferred example of the medium for culturing the transformant of which the host is bacteria belonging to the genus Escherichia is M9 medium supplemented with glucose and Casamino acids [Miller, J. Exp. Mol. Genet., 431-433, Cold Spring Harbor Laboratory, New York, 1972]. If necessary, a chemical such as 3β-indolylacrylic acid can be added to the medium thereby to activate the promoter efficiently.

Where the bacteria belonging to the genus Escherichia are used as the host, the transformant is usually cultivated at about 15 to 43° C. for about 3 to 24 hours. If necessary, the culture may be aerated or agitated.

Where the bacteria belonging to the genus Bacillus are used as the host, the transformant is cultured generally at about 30 to 40° C. for about 6 to 24 hours. If necessary, the culture can be aerated or agitated.

Where yeast is used as the host, the transformant is cultivated, for example, in Burkholder's minimal medium [Bostian, K. L. et al., Proc. Natl. Acad. Sci. USA, 77: 4505 (1980)] or in SD medium supplemented with 0.5% Casamino acids [Bitter, G. A. et al., Proc. Natl. Acad. Sci. USA., 81: 5330 (1984)]. Preferably, pH of the medium is adjusted to about 5 to 8. In general, the transformant is cultivated at about 20 to 35° C. for about 24 to 72 hours. If necessary, the culture can be aerated or agitated.

Where insect cells or insects are used as the host, the transformant is cultivated in, for example, Grace's Insect Medium [Grace, T. C. C., Nature, 195: 788 (1962)] to which an appropriate additive such as immobilized 10% bovine serum is added. Preferably, pH of the medium is adjusted to about 6.2 to about 6.4. Normally, the transformant is cultivated at about 27° C. for about 3 days to about 5 days and, if necessary, the culture can be aerated or agitated.

Where animal cells are employed as the host, the transformant is cultured in, for example, MEM medium containing about 5 to 20% fetal bovine serum [Science, 122: 501 (1952)], DMEM medium [Virology, 8: 396 (1959)], RPMI 1640 medium [J. Am. Med. Assoc., 199: 519 (1967)], 199 medium [Proc. Soc. Bio. Med., 73: 1 (1950)], etc. Preferably, pH of the medium is adjusted to about 6 to about 8. The transformant is usually cultivated at about 30° C. to about 40° C. for about 15 to 60 hours and, if necessary, the culture can be aerated or agitated.

As described above, TRAILs can be produced inside or outside the transformant cells.

TRAILs can be separated and purified from the culture obtained by culturing the transformant in accordance with publicly known methods.

When TRAILs is extracted from the bacteria or cells, the bacteria or cell collected by a publicly known method is suspended in an appropriate buffer. The bacteria or cell is then disrupted by publicly known methods such as ultrasonication, a treatment with lysozyme and/or freeze-thaw cycling, followed by centrifugation, filtration, etc to produce crude extract of the soluble protein. The buffer used for the procedures may contain a protein modifier such as urea or guanidine hydrochloride, or a surfactant such as Triton X-100™, etc.

The separation and purification of TRAILs in thus obtained soluble fraction can be carried out in accordance with publicly known methods. Such publicly known methods for separation and purification include a method utilizing difference in solubility such as salting out, solvent precipitation, etc.; a method mainly utilizing difference in molecular weight such as dialysis, ultrafiltration, gel filtration, SDS-polyacrylamide gel electrophoresis, etc.; a method utilizing difference in electric charge such as ion exchange chromatography, etc.; a method utilizing difference in specific affinity such as affinity chromatography, etc.; a method utilizing difference in hydrophobicity such as reverse phase high performance liquid chromatography, etc.; a method utilizing difference in isoelectric point such as isoelectrofocusing electrophoresis; and the like. These methods may be appropriately combined to be used.

When TRAIL or its partial peptide is in a free form, the free form can be converted into the salt by publicly known methods or modifications thereof. On the other hand, when the protein or peptide is obtained in the form of a salt, it can be converted into the free form or in the form of a different salt by publicly known methods or modifications thereof.

TRAILs produced by the transformant can be treated, prior to or after the purification, with an appropriate protein-modifying enzyme so that the protein can be subjected to addition of an appropriate modification or removal of a partial polypeptide. Examples of the protein-modifying enzyme include trypsin, chymotrypsin, arginyl endopeptidase, protein kinase, glycosidase and the like.

The presence of the thus produced TRAILs can be determined by an enzyme immunoassay or western blotting using a specific antibody.

TRAIL or its partial peptide can be synthesized by the in vitro translation with the use of a cell-free protein translation system such as rabbit reticulocyte lysate, wheat germ lysate, colon bacillus lysate, etc., comprising RNA which corresponds to DNA encoding the protein as a template. TRAIL or its partial peptide can also be synthesized by a cell-free transcription/translation system comprising RNA polymerase with the use of DNA encoding TRAIL or its partial peptide as a template. For the cell-free protein (transcription /) translation system, commercially available ones and a method known per se can be employed. In particular, the Escherichia coli extract solution can be prepared according to a method disclosed in Pratt J. M. et al, “Transcription and Translation”, Hames B. D. and Higgins S. J. eds., IRL Press, Oxford 179-209 (1984). As the commercially available cell lysate, Escherichia coli derived lysates such as E. coli S30 extract system (manufactured by Promega) and RTS 500 Rapid Tranlation System (manufactured by Roche), etc.; rabbit reticulocyte derived lysates such as Rabbit Reticulocyte Lysate System (manufactured by Promega), etc.; and wheat germ derived lysates such as PROTEIOS™(manufactured by TOYOBO), etc., can be exemplified. Among them, the wheat germ lysate is preferably used. As the method of manufacturing wheat germ lysate, for example, methods disclosed in Johnston F. B. et al, Nature, 179: 160-161 (1957), Erickson A. H. et al, Meth. Enzymol., 96: 38-50 (1996), etc., can be employed.

As a system or device for the protein synthesis, a batch method [Pratt, J. M. et al, (1984) mentions above]; continuous cell-free protein synthesis system [Spirin A. S. et al, Science, 242: 1162-1164 (1988)] in which an amino acid, energy source, etc. is continuously supplied in a reaction system; dialysis (Kikawa, et al, No. 21 Molecular Biology Society of Japan, WID6)); a double layer method (PROTEIOS™ Wheat germ cell-free protein synthesis core kit instruction manual: manufactured by TOYOBO), and the like can be exemplified. In addition, a method which comprises supplying RNA template, amino acid, energy source, etc., into a synthesis reaction system according to necessity, and eliminating a synthesized or separated material according to necessity (Japanese Patent Laid-Open No. 2000-333673) can be used.

The examples of the agonist compound against TRAILR1 or TRAILR2 include TRAIL (AMG951 (Genentech/Amgen)), an agonist (signaling) antibody against TRAILR1 (e.g., antibody having amino acid sequence shown by SEQ ID NO: 24), an agonist (signaling) antibody against TRAILR2 (CS-1008 (Daiichi Sankyo Inc.), and AMG655 (Amgen)) (see Uta Schaefer et al, Frontiers in Bioscience 12, 3813-3824, May 1, 2007). In addition, the examples of the agonist compound of TRAILR1 or TRAILR2 include an agonist to the receptor selected by screening by the use of TRAIL and TRAILR1 or TRAILR2.

The agonist (signaling) antibody to TRAILR1 or TRAILR2 may be selected from the antibody for the receptor produced by general technique for preparing antibody, employing an acrivation of TRAIL signal transduction activity (for example, activity of caspase, apoptosis induction, and the like), as a marker. The antibody may be any of polyclonal antibody and monoclonal antibody. The isotype of the antibody is not particularly limited, but it is preferably IgG, IgM or IgA, particularly preferably IgG. The antibody is not particularly subject to limitation, as long as it has at least a complementality determining region (CDR) for specifically recognizing and binding to the target antigen; in addition to the whole antibody molecule, the antibody may, for example, be a fragment such as Fab, Fab′, or F(ab′)₂, a genetically engineered conjugate molecule such as scFv, scFv-Fc, minibody, or diabody, or a derivative thereof modified with a molecule having protein stabilizing action, such as polyethylene glycol (PEG), or the like, and the like. There may be a case that the fragmentation of the antibody causes increase of the agonist (signal) transduction activity more remarkably as compared with the case of the whole antibody.

In a preferred embodiment, since the agonist antibody to TRAILR1 or TRAILR2 is used as a pharmaceutical product administered to human being, the antibody (preferably, a monoclonal antibody) is an antibody having a reduced risk of exhibiting antigenicity when administered to human being, and is specifically exemplified by a complete human antibody, a humanized antibody, a mouse-human chimeric antibody, with a complete human antibody being particularly preferred. A humanized antibody and a chimeric antibody can be produced in a genetically engineered manner, according to conventional methods. It is also possible to produce a complete human antibody from a human-human (or mouse) hybridoma, but in order to provide a large quantity of antibody stably at low costs, it is preferable to carry out the production using a human antibody-producing mouse or a phage display method.

Screening using TRAIL and TRAILR1 or TRAILR2 (hereinafter, may be collectively referred to as TRAILR) can be performed, for example, as follows.

(1) For the case of contacting labeled TRAIL with TRAILR, and for the case of contacting labeled TRAIL and a test compound with TRAILR, the amounts of labeled TRAIL binding to TRAILR are measured and compared, and thereby a compound which competes with TRAIL for binding to TRAILR, or a salt thereof, is screened.

(2) For the case of contacting labeled TRAIL with a cell producing TRAILR, or a membrane fraction thereof, and for the case of contacting labeled TRAIL and a test compound with a cell producing TRAILR, or a membrane fraction thereof, the amounts of labeled TRAIL binding to the cell or membrane fraction are measured and compared, and thereby a compound which competes with TRAIL for binding to TRAILR, or a salt thereof, is screened.

(3) For the case of contacting labeled TRAIL with TRAILR which has been expressed on the cellular membrane by culturing a transformant containing a TRAILR-encoding DNA, and for the case of contacting labeled TRAIL and a test compound with TRAILR which has been expressed on the cellular membrane by culturing a transformant containing a TRAILR-encoding DNA, the amounts of labeled TRAIL binding to TRAILR are measured and compared, and thereby a compound which competes with TRAIL for binding to TRAILR, or a salt thereof, is screened.

(4) For the case of contacting TRAIL with a cell expression TRAILR on the cellular membrane, and for the case of contacting a test compound with a cell expressing TRAILR on the cellular membrane, the cell stimulating activity via TRAILR (for example, caspase activation, apoptosis induction, etc.) is measured and compared, and thereby a compound which activates TRAIL signaling by binding to TRAILR, or a salt thereof, is screened.

(5) For the case of contacting TRAIL with TRAILR which has been expressed on the cellular membrane by culturing a transformant containing a TRAILR-encoding DNA, and for the case of contacting a test compound with TRAILR which has been expressed on the cellular membrane by culturing a transformant containing a TRAILR-encoding DNA, the cell stimulating activity via TRAILR (for example, caspase activation, apoptosis induction, etc.) is measured and compared, and thereby a compound which activates TRAIL signaling by binding to TRAILR, or a salt thereof, is screened.

Furthermore, with regard to the methods (1 or 4) to (5) above, the TRAILR agonist compounds described above can be used in place of TRAIL.

Alternatively, when the methods (1) to (3) are carried out by directly detecting the binding of a test compound to TRAILR without using TRAIL, a compound which does not compete with TRAIL for activating signaling by binding to TRAILR can also be obtained. The corresponding methods can be carried out as follows.

(1a) In the case of contacting a test compound with TRAILR, the amount of the test compound binding to TRAILR is measured, and thereby a compound which binds to TRAILR, or a salt thereof, is screened.

(2a) In the case of contacting a test compound with a cell producing TRAILR or a membrane fraction thereof, the amount of the test compound binding to the cell or membrane fraction is measured, and thereby a compound which binds to TRAILR, or a salt thereof, is screened.

(3a) In the case of contacting a test compound with TRAILR which has been expressed on the cellular membrane by culturing a transformant containing a TRAILR-encoding DNA, the amount of the test compound binding to TRAILR is measured, and thereby a compound which binds to TRAILR, or a salt thereof, is screened.

Here, the binding amount of the test compound to the TRAILR can be investigated by labeling each test compound and then measuring the amount of label bound to TRAILR, or by employing the means such as surface plasmon resonance and the like without employing labeling.

Hereinafter, the screening method of the invention will be described in more detail.

First, TRAILR1 used for the screening method of the invention may be exemplified by a protein containing the same or substantially the same amino acid sequence as the amino acid sequence represented by SEQ ID NO:18, or a partial peptide thereof, and TRAILR2 may be exemplified by a protein containing the same or substantially the same amino acid sequence as the amino acid sequence represented by SEQ ID NO:20 or 22, or a partial peptide. These proteins and cells expressing the proteins can be obtained using the same methods as described above for TRAIL. Preferably, a cellular membrane fraction of an organ of a mammal producing TRAILR is used, but since a human-derived organ is not easily available, human-derived TRAILR which has been expressed in large quantities using a recombinant, or the like is suitable for the use in screening.

Production of TRAILR is preferably carried out by expressing a DNA encoding TRAILR (for example, SEQ ID NO:17, 19 or 21) in a mammalian cell or an insect cell. As the DNA fragment encoding the desired protein portion, a cDNA is used, but the DNA fragment is not limited thereto. For example, a gene fragment or a synthetic DNA may also be used. In order to introduce a TRAILR-encoding DNA fragment into a host animal (insect) cell, and express the DNA fragment efficiently, it is preferable to incorporate the DNA fragment into a location downstream to a SV40-derived promoter, a retroviral promoter, a metallothionein promoter, a human heat-shock promoter, a cytomegalovirus promoter, an SRα promoter, a polyhedrin promoter of nuclear polyhedrosis virus (NPV), which is a baculovirus using an insect as the host, or the like.

Therefore, the TRAILR used for the screening method of the invention may be TRAILR purified according to methods that are known per se, or may be used in the form of cells producing TRAILR, or a cellular membrane fraction thereof.

For the screening method described above, in the case of using cells producing TRAILR, the cells may be fixed with glutaraldehyde, formalin or the like. The fixation method can be carried out according to methods that are known per se.

The cell producing TRAILR refers to a host cell expressing TRAILR, and the host cell may be exemplified by yeast, insect cells, animal cells, or the like.

The cellular membrane fraction refers to a fraction which is obtained by methods that are known per se, after disrupting cells, and which contains a large quantity of cellular membrane. The cell disrupting method may be exemplified by a method of crushing cells with a Potter-Elvehjem type homogenizer, disruption by means of a Waring Blender or Polytron (manufactured by Kinematica), disruption by sonication, disruption by discharging cells through a narrow nozzle while pressurizing with a French press or the like, or the like. The fractionation of cellular membrane is mainly performed using a fractionation method using centrifugal force, such as a fractionation centrifugation method, a density gradient centrifugation or the like. For example, a cell disruption liquid is centrifuged at a low speed (500 rpm to 3000 rpm) for a short time (typically, about 1 to 10 minutes), and the supernatant is further centrifuged at a higher speed (15,000 rpm to 30,000 rpm) typically for 30 minutes to 2 hours. The obtained precipitate is used as the membrane fraction. The membrane fraction contains large quantities of expressed TRAILR, membrane components such as cell-derived phospholipids, membrane proteins and the like.

The TRAILR amount in cells producing TRAILR or cell membrane fraction thereof is preferable to be 10³ to 10⁸ molecules per cell, more preferably 10⁵ to 10⁷ per cell. Additionally, the more the expression level, the higher the TRAILR binding activity (specific activity) per cell membrane fraction, therefore it become possible not only to develop a sensitive screening system but to measure a large amount of samples in the same lot.

In order to carry out the above (1) to (3) for screening compounds binding to TRAILR competitively with TRAIL, for instance, appropriate cell membrane fraction including TRAILR and labeled TRAIL are needed.

A cell membrane fraction including TRAILR is desirably a natural cell membrane fraction including TRAILR or recombinant cell membrane fraction including TRAILR which has the equivalent activity to the natural type. Here, ‘equivalent activity’ refers to equivalent TRAIL binding activity, an effect of signal transduction and the like.

For the labeled TRAIL, for example, those labeled with [³H], [³²P], [¹²⁵I] and [¹⁴C] in accordance with a usual method are employed.

Specifically, so as to screen a compound which binds to TRAILR competing for TRAIL, first, TRAILR preparation is prepared by suspending cells producing TRAILR or membrane fractions in a buffer appropriate for screening. Any buffer can be used so long as it does not interfere with the binding between the protein of TRAIL and TRAILR, such buffers including a phosphate buffer or a Tris-HCl buffer, having pH of approximately 4 to 10 (preferably pH of approximately 6 to 8), etc. For the purpose of minimizing non-specific binding, a surfactant such as CHAPS, Tween-80™ (manufactured by Kao-Atlas Inc.), digitonin, deoxycholate, etc. may be added to the buffer. Besides, for the purpose of reducing the degradation of the TRAILR by a protease, a protease inhibitor such as PMSF, leupeptin, E-64 (manufactured by Peptide Institute, Inc.), pepstatin, etc. may also be added. A predetermined quantity (5,000 to 500,000 cpm) of labeled TRAIL is added to 0.01 to 10 ml of the TRAILR suspension together with a test compound at 10⁻⁴ M to 10⁻¹⁰ M. To find the amount of non-specific binding (NSB), a reaction tube containing a large excess of an unlabeled ligand is also provided. The reaction is carried out at 0 to 50° C., preferably 4 to 37° C. for 3 to 30 hours, preferably 20 to 24 hours. After completion of the reaction, the reaction mixture is filtrated through a glass fiber filter paper or the like and washed with a suitable volume of the same buffer. The residual radioactivity remaining on the glass fiber filter paper is then measured by means of a liquid scintillation counter or a gamma-counter. With the count (B₀-NSB) after subtracting the amount of non-specific binding (NSB) from the count (B₀) without an antagonistic substance (a test compound) being taken as 100%, a test compound giving a specific binding value (B-NSB) of not more than 50% of the count, which is a compound capable of binding by competing for TRAILR can be selected as a candidate agonist compound.

In order to carry out the methods (4) and (5) for screening the compound activating a TRAIL signal by binding to TRAILR, for example, cell-stimulating activity (for example, caspase activity, apoptosis induction etc.) via TRAILR can be assayed by publicly known methods or a commercially available measuring kit.

Specifically, first, cells producing TRAILR are cultivated in a multi-well plate or the like. For screening, fresh medium or appropriate buffer atoxic to the cells is provided in advance, and a test compound or the like is added to incubate for a predetermined time. Thereafter, cells are extracted or supernatant is recovered, and thus given product is quantified according to each method. When assaying the production of substance serving as an indication of cell-stimulating activity is difficult due to a degradative enzyme contained in the cells, the assay may be performed by adding an inhibitor for the degradative enzyme.

In order to perform screening by measuring the apoptosis inducing activity, appropriate cells expressing TRAILR on the cell membrane are need. The cells expressed with TRAILR are desirably cell lines producing natural TRAILR, cell lines expressing the aforementioned recombinant TRAILR, or the like.

Examples of the test compound include peptide, proteins, nonpeptidic compounds, synthetic compounds, fermentation products, cell extracts, vegetable extracts, animal tissue extracts, etc. and these compounds may be novel compounds or publicly known compounds.

A screening kit for the compound binding to TRAILR competitively with TRAIL or a salt thereof includes TRAILR, cells producing TRAILR, or cell membrane fraction thereof, and the like.

Examples of the screening kit of the invention include followings.

### (I) Reagents for Screening

### (1) Assay Buffer and Wash Buffer

Hanks' Balanced Salt Solution (manufactured by Gibco) supplemented with 0.05% of bovine serum albumin (manufactured by Sigma).

The buffers may be sterilized by filtration through a filter with a 0.45 μm pore size and stored at 4° C., or may be prepared at use.

### (2) TRAILR Preparation

CHO cells expressing TRAILR are subcultured at 5×10⁵ cells/well on a 12-well plate at 37° C. under 5% CO₂ and 95% air for 2 days.

### (3) Labeled TRAIL

TRAIL (includes analog), which is labeled with commercially available [³H], [³²P], [¹²⁵I], [¹⁴C] and the like and is in the form of an aqueous solution, is stored at 4° C. or −20° C., and is diluted to 1 μM by adding assay buffer at use.

### (4) TRAIL Standard Solution

TRAIL (includes analog) is dissolved in PBS containing 0.1% of bovine serum albumin (manufactured by Sigma) to make the volume 1 mM and then stored at −20° C.

### (II) Procedures for Assay

(1) The CHO cells expressing TRAILR, cultivated in a 12-well tissue culture plate, are washed twice with 1 ml of the assay buffer, followed by addition of 490 μl of the assay buffer to each well.
(2) After 5 μl of a test compound solution of 10⁻³ to 10⁻¹⁰ M is added, 5 μl of the labeled TRAIL is added thereto followed by reacting at room temperature for an hour. In order to find the amount of the non-specific binding, 5 μl of the TRAIL of 10⁻³ M is added to thereto in place of the test compound.
(3) The reaction solution is removed, and the plate is washed three times with 1 ml each of the wash buffer. The labeled TRAIL bound to the cells or the plate is dissolved in 0.2N NaOH-1% SDS and the solution is mixed with 4 ml of liquid scintillator A (manufactured by Wako Pure Chemical Industries, Ltd.).
(4) Radioactivity is measured with a liquid scintillation counter (manufactured by Beckman) and Percent Maximum Binding (PMB) is calculated in accordance with the following equation.

PMB=[(B−NSB)/(B₀−NSB)]×100

### PMB: Percent Maximum Binding

B: value when the sample is added

### NSB: Non-specific Binding

B₀: maximum binding amount

In the case of employing the apoptosis induction through the cell stimulating activity as an index, for example, examination can be carried out by detecting membrane blebbing, reduction in cell size, chromatin condensation, DNA fragmentation or the like, by means of morphological observation using an optical microscope, a phase contrast microscope, a fluorescent microscope or the like. For example, cells are cultured in a multi-well plate, cells detached from the plate or cells which have undergone membrane blebbing are counted under a microscope, and the ratio (%) of dead cells among the entire cells within a field of vision is calculated. Observation is made in several fields of vision, and the average value obtained therefrom is taken as the cell death induction rate. Furthermore, the cell death induction rate can also be deduced by staining dead cells using a dye such as Trypan Blue, Erythrosin B, Negrosin, Eosin Y, fluorescein diacetate, Acridine Orange, ethidium bromide or the like, and counting the stained dead cells under a microscope. Moreover, DNA may be stained using a fluorescent dye such as DAPI, Hoechst 33342 or the like, and cells which are undergoing chromatin condensation may be counted under a fluorescent microscope. Alternatively, dUTP labeled with biotin, a fluorescent dye or the like is added to the 3′-terminal of a fragmented DNA using Terminal Deoxynucleotidyl Transferase (TdT) (TUNEL method), and the number of intensely stained cells is counted using an optical microscope, a fluorescent microscope or the like, whereby the apoptosis rate can be induced.

The apoptosis induction ratio can be assayed by counting the number of cells reduced in size or fragmented by assessing the cell size distribution with the use of a particle-size measuring apparatus (e.g., Coulter multisizer, etc.). The dead-cell induction ratio can also be assayed by identifying the alive/dead cells by detecting the reduction in cell size and the fragmentation to apoptotic bodies by using flow cytometry (FACS).

Further, apoptosis can be biochemically detected by extracting chromosomal DNA from a cell using a conventional method, and carrying out a gel electrophoresis to measure the level of DNA fragmentation using a densitometer or the like. The dead-cell induction ratio can also be assayed by measuring the absorbance decrease in 570-630 nm using a microplate reader as 3-(4,5-dimethyl-thiazol-2-yl)-2,5-diphenyl tetrazolium bromide (MTT) is reduced to formazan by alive cells.

As a result of the above screening, when cell-stimulating activity (for example, caspase activity, apoptosis inducing activity, etc.) is increased in the presence of a test compound, the compound can be selected as a compound having cell-stimulating activity via an interaction between TRAIL and TRAILR, that is, as an agonist compound to TRAILR.

TRAIL signal activator provides therapeutic benefit to prevent and/or remedy cancers in TRAIL signal activator sensitive patient selected as described above, for example, breast cancer (e.g., invasive breast cancer, noninvasive breast cancer, inflammatory breast cancer, etc.), prostate cancer (e.g., hormone-dependent prostate cancer, hormone-independent prostate cancer, etc.), pancreas cancer (e.g., pancreas cancer, etc.), stomach cancer (e.g., papillary adenocarcinoma, mucous gland carcinoma, adenosquamous carcinoma, etc.), lung cancer (e.g., non-small cell lung cancer, small cell lung cancer, malignant mesothelioma, etc.), colon cancer (e.g., gastrointestinal stromal tumor, etc.), rectal cancer (e.g., gastrointestinal stromal tumor, etc.), large intestine cancer (e.g., familial colon cancer, hereditary nonpolyposis colon cancer, gastrointestinal stromal tumor, etc.), small intestine cancer (e.g., non-Hodgkin's lymphoma, gastrointestinal stromal tumor, etc.), esophagus cancer, duodenal cancer, tongue cancer, pharynx cancer (e.g., nasopharyngeal cancer, oropharynx cancer, hypophrynx cancer etc.), salivary gland cancer, brain tumor (e.g., pineal astrocytoma, pilocytic astrocytoma, diffuse astrocytoma, anaplastic astrocytoma, etc.), neurilemmonma, liver cancer (e.g., primary hepatic cancer, extrahepatic bile duct cancer, etc.), kidney cancer (e.g., renal cell cancer, transitional cell cancer of the renal pelvis and ureter, etc.), bile duct cancer, endometrial cancer, uterine cervix cancer, ovary cancer (e.g., epithelial ovarian cancer, extragonadal germ cell tumor, ovarian germ cell tumor, ovarian low-malignant potential tumor, etc.), bladder cancer, urethral cancer, skin cancer (e.g., intraocular (eye) melanoma, Merkel cell carcinoma, etc.), angioma, malignant lymphoma, malignant melanoma, thyroid cancer (e.g., medullary cancer, etc.), parathyroid cancer, nasal cancer, paranasal cancer, bone tumor, (e.g., osteosarcoma, Ewing's tumor, uterine sarcoma, soft tissue sarcoma, etc.), hemangiofibroma, retinal sarcoma, penis cancer, testicular tumor, solid tumor in children (e.g., Wilm's tumor, childhood kidney tumor, etc.), Kaposis's sarcoma, AIDS-associated (related) Kaposis's sarcoma, tumor of maxillary sinus, fibrous histiocytoma, leiomyosarcoma, rhabdomyosarcoma, leukemia (e.g., acute myeloid leukemia, acute lymphoblastic leukemia, etc.), and the like, preferably breast cancer, large intestine cancer, lung cancer, stomach cancer, pancreas cancer, prostate cancer, ovary cancer, blood cancer. Agents comprising TRAIL signal activator are low toxic, and can be administered as they are in the form of liquid preparation, or as pharmaceutical compositions of suitable preparations to human or mammals (e.g., mouse, rats, rabbits, sheep, swine, bovine, feline, canine, simian, etc.) orally or parenterally (e.g., intravascularly, subcutaneously, etc.).

The TRAIL signal activator may be administered in itself, or may be administered as an appropriate pharmaceutical composition. The pharmaceutical composition used for the administration may contain the TRAIL signal activator as well as a pharmacologically acceptable carrier, a diluent or excipient. Such a pharmaceutical composition is provided in the form of pharmaceutical preparations suitable for oral or parenteral administration.

Examples of the composition for parenteral administration are injectable preparations, suppositories, etc. The injectable preparations may include dosage forms such as intravenous, subcutaneous, intracutaneous and intramuscular injections, drip infusions, etc. These injectable preparations may be prepared by methods publicly known. For example, the injectable preparations may be prepared by dissolving, suspending or emulsifying the antibody of the present invention described above, the low molecular compound or the salts thereof in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mols) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injectable thus prepared is usually filled in an appropriate ampoule. The suppository used for rectal administration may be prepared by blending the antibody described above or the salts thereof with conventional bases for suppositories.

The composition for oral administration includes solid or liquid preparations, particularly, tablets (including dragees and film-coated tablets), pills, granules, powdery preparations, capsules (including soft capsules), syrup, emulsions, suspensions, etc. Such a composition is manufactured by publicly known methods and contains a carrier, a diluent or an excipient conventionally used in the field of pharmaceutical preparations. Examples of the carrier or excipient for tablets are lactose, starch, sucrose mangnesium stearate.

Advantageously, the pharmaceutical compositions for oral or parenteral use described above are prepared into pharmaceutical preparations with a unit dose formed to fit a dose of the active components. Such unit dose preparations include, for example, tablets, pills, capsules, injections (ampoules) and suppositories. The antibody or the low molecular compound is generally contained in 5 to 500 mg per dosage unit form, in 5 to 100 mg especially in the form of injection, and in 10 to 250 mg for the other forms.

The dose of the above medicine containing the TRAIL signal activator varies depending on effect, target disease, subject to be administered, conditions, route of administration etc. In oral administration to a normal adult (60 kg body weight), the daily dose of the TRAIL signal activator is generally about 0.1 to 100 mg, preferably about 1.0 to 50 mg, and more preferably about 1.0 to 20 mg. In parenteral administration, the dose of the TRAIL signal activator varies depending on target disease, subject to be administered, conditions, route of administration etc. In case of administering as an injection, it is advantageous to administer to a normal adult (60 kg body weight) the TRAIL signal activator intravenously in a daily dose of about 0.01 to 30 mg, preferably about 0.1 to 20 mg, and more preferably about 0.1 to 10 mg. For other animal species, the corresponding dose as converted per 60 kg body weight can be administered.

Each composition described above may further contain other active ingredients (for example, other anticarcinogenic agents) unless formulation causes any adverse effect on the TRAIL signal activator. Furthermore, the each composition may be used in combination with other agents, for example, anticarcinogenic agents.

The dose of the active ingredient (for example, other anticarcinogenic agents) varies depending on effect, target disease, subject to be administered, conditions, route of administration etc. In oral administration to a normal adult (60 kg body weight), the daily dose of the active ingredient is generally about 0.1 mg to about 100 mg, preferably about 1.0 to about 50 mg, and more preferably about 1.0 to about 20 mg. In parenteral administration, the single dose of the active ingredient also varies depending on target disease, subject to be administered, conditions, route of administration etc. In case of administering as an injection, it is advantageous to administer to a normal adult (60 kg body weight) the active ingredient intravenously in a daily dose of about 0.01 to about 30 mg, preferably about 0.1 to about 20 mg, and more preferably about 0.1 to about 10 mg. For other animal species, the corresponding dose as converted per 60 kg body weight can be administered.

### 2. Method of Diagnosing Sensitivity to TRAIL Signal Activator

The preventive/remedy agent for cancer of the invention containing a TRAIL signal activator is characterized in that the agent is administered to a TRAIL signal activator sensitive patient who is screened by using the expression or activity of TRAIL signal activator sensitivity markers in a sample collected from a cancer patient, as an index. Therefore, the invention provides a method of diagnosing the sensitivity of a patient to a TRAIL signal activator, using the TRAIL signal activator sensitivity markers of the invention.

Preferably, the invention provides a method of diagnosing the sensitivity of a cancer patient to a TRAIL signal activator, the method comprising examining the expression or activity of STK17B, LOC93349 and CASP8 in a sample collected from the patient. The type of cancer to which the diagnostic method is applicable, the sample to be collected from a patient, the method of measuring the level of expression or activity of the respective TRAIL signal activator sensitivity markers, the technique of determining the sensitivity/insensitivity to a TRAIL signal activator from the obtained measurement values, and the like are all the same as described with regard to the TRAIL signal activator-containing preventive/remedy agent for cancer.

As a preferred determination technique, there may be mentioned a method of setting an upper reference value and a lower reference value for each of the markers according to the Method 2 described above, and scoring three markers based on the same weighting. For example, given that a +1 point is scored if the level of expression or activity (of the learning cancer cells which is relative to the level of expression or activity of the reference cancer cells) of STK17B, LOC93349 or CASP8 in a sample collected from a test subject is equal to or greater than the upper reference value; a zero point is scored if the level of expression or activity is equal to or greater than the lower reference value and also less than the upper reference value; and a −1 point is scored if the level of expression or activity is less than the lower reference value, the sum of the points for each of the markers STK17B, LOC93349 and CASP8 is determined.

As a result, if the sum is a positive value (+1 to +3), the test subject is determined to be TRAIL signal activator sensitive. On the contrary, if the sum is a negative value (−1 to −3), the test subject is determined to be TRAIL signal activator insensitive. Also, if the sum is zero, the result can be said to be indeterminable.

If the test subject is a patient of breast cancer or colon cancer, it is more preferable to use AIM1, in addition to the three markers described above, as another TRAIL signal activator sensitivity marker. The diagnostic method of the invention is characterized in that the determination of insensitivity by means of AIM1 is given the priority to the comprehensive determination by means of the other three markers. That is, if the determination result obtained using AIM1 alone is insensitive (the level of expression or activity of AIM1 is equal to or greater than the reference value), regardless of whether the determination result obtained from the other three markers is sensitive or insensitive, the test subject is determined to be TRAIL signal activator insensitive. On the other hand, if the determination result obtained using AIM1 alone is indeterminable, that is, the level of expression or activity of AIM1 is less than the reference value, the comprehensive determination by means of the other three markers is given the priority, and if the result is sensitive, the subject is determined to be TRAIL signal activator sensitive.

Preferably, the reference value (cut-off value) for the determination of sensitivity/insensitivity to AIM1 is set according to the method described above.

### 3. Diagnostic Agent for TRAIL Signal Activator Sensitivity

The present invention also provides a diagnostic agent, which is suitable for carrying out the diagnostic method of the invention described above. The diagnostic agent for TRAIL signal activator sensitivity according to the invention includes a reagent capable of detecting a TRAIL signal activator sensitivity marker, preferably each the expression or the activity of STK17B, LOC93349, and CASP8. The reagent ingredient varies depending on the detection target. The ingredient is a nucleic acid capable of detecting mRNA of signal activator sensitivity marker gene when a detection target is the mRNA, the ingredient is an antibody to a protein of the marker when a detection target is the protein, and the ingredient is a reagent for activity measurement when detecting the activity of the protein.

Examples of the nucleic acid, which can detect a TRAIL signal activator sensitivity marker gene, particularly mRNA of STK17B, LOC93349 and CASP8, include the following (a) and/or (b):

(a) a nucleic acid hybridizable to the nucleic acid having (i) a base sequence represented by any of SEQ ID NO n (n=3, 5, 7 and 9) or (ii) a base sequence of ortholog of human genome having the above mentioned base sequence (i) in other mammals, under high stringent conditions;

(b) a nucleic acid hybridizable to the nucleic acid having a base sequence complimentary to the base sequence (i) or (ii) under high stringent conditions.

Here, “high stringent conditions” means the conditions for the nucleic acid having base sequences represented by SEQ ID NO n to be hybridizable to the nucleic acid which has a base sequence having in an overlapping region at least about 80% complementality, preferably at least about 90% complementality, and more preferably at least about 95% complementality. The high stringent conditions are, for example, those in 6×SSC (sodium chloride/sodium citrate) at 45° C. for a hybridization reaction and then in 0.2×SSC/0.1% SDS at 65° C. for performing one or more washing, etc.

The nucleic acid may exemplified by the nucleic acid for probe or the oligonucleotide for primer described in the section of preventive/remedy for cancer comprising TRAIL signal activator. The nucleic acid functioning as a probe capable of detecting the expression of TRAIL signal activator sensitivity marker gene can be obtained by:

amplifying a nucleic acid having desired length by PCR method with the use of cDNA or genomic DNA derived from mammalian (e.g., human, simian, mouse, rat, canine, bovine, horse, swine, sheep, goat, feline, rabbit, hamster, guinea swine, etc.) cells [for example, hepatocytes, splenocytes, nerve cells, glial cells, β cells of pancreas, bone marrow cells, mesangial cells, Langerhans' cells, epidermic cells, epithelial cells, goblet cells, endothelial cells, smooth muscle cells, fibroblasts, fibrocytes, myocytes, fat cells, immune cells (e.g., macrophages, T cells, B cells, natural killer cells, mast cells, neutrophils, basophils, eosinophils, monocytes), megakaryocytes, synovial cells, chondrocytes, bone cells, osteoblasts, osteoclasts, mammary cells, or interstitial cells; or the corresponding precursor cells, stem cells, cancer cells, etc.]; or any tissues where such cells are present [for example, brain or each part of brain (e.g., olfactory bulb, amygdaloid nucleus, basal ganglia, hippocampus, thalamus, hypothalamus, cerebral cortex, medulla oblongata, cerebellum), spinal cord, hypophysis, stomach, pancreas, kidney, liver, gonad, thyroid, gall-bladder, bone marrow, adrenal gland, skin, lung, gastrointestinal tract (e.g., large intestine and small intestine), blood vessel, heart, thymus, spleen, submandibular gland, peripheral blood, prostate, testis, ovary, placenta, uterus, bone, joint, adipose tissue, skeletal muscle, etc.], as a template; or

by cloning the TRAIL signal activator sensitivity marker gene or cDNA from the cDNA or genomic DNA library derived from the above-described cells and tissues in accordance with a colony or plaque hybridization, or the like, and if necessary using a restriction enzyme to obtain a fragment having appropriate length. The hybridization can be carried out, for example, according to the method described in Molecular Cloning, 2nd (described above). Alternatively, the nucleic acid can also be obtained chemically synthesizing a part or whole of the base sequence and/or its complementary sequence with the use of a commercially available DNA/RNA automatic synthesizer, on the basis of each base sequence information represented by SEQ ID NO: n (n=3, 5, 7, and 9). In addition, a chip on which the nucleic acid is solid-phased can be prepared by directly synthesizing the nucleic acid, in situ (on chip) on the solid phase such as silicon, glass, and the like.

A nucleic acid, which functions as a primer capable of amplifying a part of or all of the transcript product of TRAIL signal activator sensitivity marker gene, can be obtained by a chemical synthesis with the use of a part of the base sequence and a part of the complementary sequence thereof on the basis of each base sequence information represented by SEQ ID NO: n (n=3, 5, 7 and 9) using a commercially available automated DNA/RNA synthesizer.

The nucleic acid capable of detecting the expression of TRAIL signal activator sensitivity marker gene, can be provided as a solid in a dry form or in alcohol precipitation, or in a dissolved form in water or adequate buffer (e.g., TE buffer or the like). In case of being employed as a labeled probe, the nucleic acid can be preliminarily labeled with any of above-mentioned labeling substances, or can be provided separately to the labeling substance so as to be labeled at use.

Alternatively, the nucleic acid can be provided in an appropriately immobilized form as a solid phase. Examples of the solid phase include glass, silicon, plastic, nitrocellulose, nylon, polyvinylidene difluoride, and the like, but the invention is not limited thereto. For the immobilization, there may be employed a method of crosslinking the nucleic acid to which a functional group such as an amino group, an aldehyde group, an SH group, or a biotin group is preliminarily introduced with the solid phase also to which a functional group capable of reacting with the nucleic acid is introduced, through a covalent bond between two functional groups, or immobilizing the nucleic acid using an electrostatic binding, by subjecting a solid phase to a polycation coating, but the invention is not limited thereto.

A nucleic acid probe provided as an immobilized solid phase can be preferably exemplified by ArrayPlate™ provided by High Throughput Genomics, Inc. For ArrayPlate™, various nucleic acid probes are immobilized in the state of being placed in a regular manner on the bottom of each well in the 96-well plate (for example, 4×4 array). The hybridization reaction between the probe and the target nucleic acid can be carried out not on the surface of a solid phase but in a liquid phase by mediating, as a spacer, a nucleic acid having one end hybridizable to the probe and the other end hybridizable to the target nucleic acid, and thus a quantitative measurement of the target nucleic acid becomes possible. Accordingly, fluctuation in the expression of various TRAIL signal activator sensitivity marker genes can be detected at once in a single well, and when a sufficient quantitativeness is obtained, there is an advantage of giving a greater efficiency than real-time PCR which detects fluctuation in expression of each marker gene.

The nucleic acid included in each reagent is preferable to be constituted in a manner to allow the detection of the expression of TRAIL signal activator sensitivity marker gene by the same method (e.g., northern blotting, dot blotting, DNA array technique, quantitative RT-PCR, etc.).

The constitution of the reagent of the invention is exemplified by those in which the above-mentioned reagents are separately provided [for example, in the case where the nucleic acid functions as a labeled probe (particularly, in case of dot-blot assay) or a primer for PCR (particularly, real-time quantitative PCR), etc.]; those in which a nucleic acid capable of detecting the expression of TRAIL signal activator sensitivity marker gene is provided in a manner of being contained in the same reagent [for example, in the case where the nucleic acid functions as a probe for PCR (particularly, in the case of distinguishing each marker gene by a size or the like of the amplification product) or labeling (particularly, in the case of distinguishing each marker gene by a size of the transcription product by Northern blot assay), etc.]; those in which a nucleic acid capable of detecting the expression of different TRAIL signal activator sensitivity marker gene is provided in a manner of being immobilized on different areas in the same solid phase [for example, in the case of functioning as a probe for hybridization with labeled cRNA or the like, etc.]; or the like, but the invention is not limited thereto.

When the diagnostic reagent of the invention includes a reagent containing an antibody to TRAIL signal activator sensitivity marker protein as a component, examples of the antibody can be mentioned by an antibody to each marker protein, which can be obtained in the same manner to antiTRAILR antibody described in the section of preventive/remedy for cancer containing TRAIL signal activator. In this case, it is not considered whether the antibody is an agonist antibody or neutralizing antibody.

In the case where the diagnostic agent of the invention is for detecting the activity of TRAIL signal activator sensitivity marker protein, examples of the reagent include a reagent which is needed to measure the activity of each marker protein described in the section of preventive/remedy for cancer containing TRAIL signal activator.

In the case where the diagnostic agent of the invention is used for diagnosing TRAIL signal activator sensitivity in patients suffering from breast cancer or colon cancer, it is preferable to further include a reagent capable of detecting the expression or activity of AIM1, in addition to a reagent capable of detecting the expression or activity of STK17B, LOC93349 and CASP8.

The reagent constituting the diagnostic agent of the invention may further contain, in addition to the nucleic acid or antibody which enables detecting the expression of the TRAIL signal activator sensitivity markers, an other substance which is required in a reaction for detecting the expression of the markers, and does not have any adverse effect on the reaction when stored in a co-existing state. Alternatively, the reagent may be provided together with a separate reagent which contains a substance required in the reaction for detecting the expression of the TRAIL signal activator sensitivity markers. For example, if the reaction for detecting the expression of the TRAIL signal activator sensitivity markers is a PCR reaction, the other substance may be exemplified by a reaction buffer solution, dNTPs, heat-resistant DNA polymerase, or the like. In the case of using competitive PCR or real time PCR, a competitor nucleic acid, a fluorescent reagent (intercalator, fluorescent probe or the like as described above) or the like may be further contained. Also, if the reaction for detecting the expression of the TRAIL signal activator sensitivity markers is an antigen-antibody reaction, the other substance may be exemplified by a reaction buffer solution, a competitor antibody, a labeled secondary antibody (for example, in the case where the primary antibody is a rabbit anti-human TRAIL signal activator sensitivity marker antibody, a mouse anti-rabbit IgG labeled with peroxidase or alkaline phosphatase, etc.), a blocking solution, or the like.

### 4. Medicine Containing Regulator for TRAIL Signal Activator Sensitivity Related Factor

The TRAIL signal activator sensitivity marker of the invention is subject to a change in the sensitivity to the TRAIL signal activator if, for example, the expression of the markers in cancer cells is suppressed by using siRNA or the like (for example, when the expression or activity of AIM1 is inhibited, or when the expression or activity of STK17B, LOC93349, CASP8, SP110, NOD27 or RHOBTB3 is enhanced, the sensitivity to the TRAIL signal activator is increased). Therefore, these markers are not simple diagnostic markers for the sensitivity to a TRAIL signal activator, but are factors which determine the sensitivity/insensitivity to a TRAIL signal activator in the cancer cells of a patient (TRAIL signal activator sensitivity related factors). Therefore, the markers are promising candidate for remedy target, purported to enhance the efficacy of the TRAIL signal activator by increasing the sensitivity of a cancer patient to the TRAIL signal activator.

Accordingly, the present invention provides a way of preventing or treating cancer in animals either by (A) inhibiting the expression or activity of AIM1 or (B) enhancing the expression or activity of STK17B, LOC93349, CASP8, SP110, NOD27 or RHOBTB3, particularly provides a method of increasing the TRAIL signal activator sensitivity.

Specifically, the above described method (A) includes the administration of a substance inhibiting the expression or activity of AIM1 (referred to as “AIM1 inhibitor”) to mammals in need of prevention and/or treatment against cancer, preferably cancer less sensitive or insensitive to TRAIL signal activator. Accordingly, the invention also provides preventive or remedy for cancer containing the AIM1 inhibitor.

In the present invention, a “substance inhibiting the expression of AIM1” may act at any level such as AIM1 gene transcription, posttranscription adjustment, translation to proteins, and posttranslation modification. Therefore, substances inhibiting the expression of AIM1 include, for example, a substance inhibiting transcription of the AIM1 gene, substance inhibiting the processing from early transcription products to mRNA, a substance inhibiting transport of mRNA to cytoplasm, a substance promoting degradation of mRNA, a substance inhibiting translation from mRNA to protein, a substance inhibiting posttranslation modification of early AIM1 translation products, and the like. The substances are preferably used at any level, but in the sense of directly inhibiting the production of AIM1 protein, the substance inhibiting translation from mRNA to protein is preferably employed.

A substance which can specifically inhibit translation from mRNA of AIM1 to protein may be preferably mentioned by a nucleic acid comprising a base sequence complementary or substantially complementary to the mRNA base sequences, or a part thereof.

The ‘base sequence substantially complementary to the base sequence of AIM1 mRNA’ refers to the base sequence having complementarity in an extent of inhibiting the translation by binding to a target sequence of the mRNA, under physiological conditions in mammalian body. Specifically, it is, for example, the base sequence having at least about 80% homology, preferably at least about 90% homology, and more preferably at least about 95% homology to the base sequence completely complimentary to the mRNA base sequence (that is, base sequence of mRNA complementary strand) in an overlapping region.

Herein, ‘homology of the base sequences’ can be measured under the following conditions (an expectation value=10; gaps are allowed; filtering=ON; match score=1; mismatch score=−3) using a homology scoring algorithm NCBI BLAST (National Center for Biotechnology Information Basic Local Alignment Search Tool).

More specifically, the complementary or substantially complementary base sequence to the base sequence of AIM1 mRNA can be exemplified by base sequences complementary or substantially complementary to (a) the base sequence represented by SEQ ID NO: 1, or (b) base sequences which are (i) hybridizable to the complementary strand of the base sequence represented by SEQ ID NO:1 under high stringent conditions, and (ii) encodes the protein having substantially the same activity as the protein comprising the amino acid sequence represented by SEQ ID NO: 2. Herein, the term ‘substantially the same activity’ refers to the same meaning as mentioned before. The high stringent conditions are, for example, those in 6×SSC (sodium chloride/sodium citrate) at 45° C. for a hybridization reaction and then in 0.2×SSC/0.1% SDS at 65° C. for performing one or more washing, etc.

As described above, AIM1 mRNA is preferably human AIM1 mRNA comprising the base sequence represented by SEQ ID NO: 1 or the homolog thereof in other mammals, or further natural splicing variant or allele variant thereof.

The “part of the base sequence complimentary or substantially complimentary to the base sequence of AIM1 mRNA” is not particularly limited in the length and the position thereof as long as it is those capable of specifically linking to AIM1 mRNA and inhibiting the translation to a protein from the mRNA, but from the viewpoint of sequence specificity, it includes bases complimentary or substantially complimentary to the target sequence of at least 10 or more, preferably about 15 or more, and more preferably about 20 or more.

In specific, the nucleic acid comprising base sequences complimentary or substantially complimentary to the base sequence of AIM1 mRNA or a part thereof can be preferably exemplified by any of the following (a) to (c):

(a) antisense nucleic acid for AIM1 mRNA
(b) siRNA for AIM1 mRNA; and
(c) nucleic acid capable of producing siRNA for AIM1 mRNA.
(a) Antisense Nucleic Acid for AIM1 mRNA

The “antisense nucleic acid for AIM1 mRNA” in the invention is a nucleic acid comprising a base sequence complimentary or substantially complimentary to the base sequence of mRNA or a part thereof, which has a function of inhibiting the protein synthesis by binding with the target mRNA to form a specifically stable duplex.

Examples of the antisense nucleic acid include polydeoxyribonucleotides containing 2-deoxy-D-ribose, polyribonucleotides containing D-ribose, any other type of polynucleotides which are N-glycosides of a purine or pyrimidine base, or other polymers containing non-nucleotide backbones (e.g., commercially available protein nucleic acids and synthetic sequence-specific nucleic acid polymers) or other polymers containing nonstandard linkages (provided that the polymers contain nucleotides having such a configuration that allows base pairing or base stacking, as is found in DNA or RNA), etc. The antisense nucleic acid may be double-stranded DNA, single-stranded DNA, double-stranded RNA, single-stranded RNA, or a DNA:RNA hybrid, and may further include unmodified polynucleotides (or unmodified oligonucleotides), those with publicly known types of modifications, for example, those with labels known in the art, those with caps, methylated polynucleotides, those with substitution of one or more naturally occurring nucleotides by their analogue, those with intramolecular modifications of nucleotides such as those with uncharged linkages (e.g., methyl phosphonates, phosphotriesters, phosphoramidates, carbamates, etc.) and those with charged linkages or sulfur-containing linkages (e.g., phosphorothioates, phosphorodithioates, etc.), those having side chain groups such as proteins (e.g., nucleases, nuclease inhibitors, toxins, antibodies, signal peptides, poly-L-lysine, etc.), saccharides (e.g., monosaccharides, etc.), those with intercalation compounds (e.g., acridine, psoralen, etc.), those containing chelator compounds (e.g., metals, radioactive metals, boron, oxidative metals, etc.), those containing alkylating agents, those with modified linkages (e.g., α anomeric nucleic acids, etc.), and the like. Herein the terms “nucleoside”, “nucleotide”, and “nucleic acid” are used to refer to those containing not only the purine and pyrimidine bases, but also other heterocyclic bases, which have been modified. Such modified substances may include methylated purines and pyrimidines, acylated purines and pyrimidines, and other heterocyclic rings. Modified nucleosides and modified nucleotides may also include those of which the sugar moiety is modified, for example, one or more hydroxyl groups may be substituted with a halogen atom(s), an aliphatic group(s), etc., or may be converted into the functional groups such as ethers, amines, or the like.

As described above, the antisense nucleic acid may be DNA or RNA, or DNA/RNA chimera. When the antisense nucleic acid is DNA, the RNA:DNA hybrid formed by the target RNA and the antisense DNA is recognized by endogenous RNase H, thereby directing the selective degradation of the target RNA. Therefore, in the case of antisense DNA mediating the degradation with RNase H, the target sequence may be the sequence in an intron-part in early translation product of AIM1 gene, in addition to the sequence in mRNA. In the case of human, the intron sequence can be determined by, for example, comparing the genome sequence in the 6q21 region of chromosome 6 where the AIM1 gene is present with the human AIM1 cDNA base sequence represented by SEQ ID NO: 1, using a homology searching program such as BLAST or FASTA.

Target region of the antisense nucleic acid of the invention is not particularly limited in its length as long as the translation into a AIM1 protein is inhibited as a result of hybridization of the antisense nucleic acid, and it may be a whole sequence or a partial sequence of mRNA encoding the protein which can be exemplified by a short strand of about 10 bases and a long strand of mRNA or whole sequence of early transcription product. In consideration of a simple synthesis, antigenic problem, and transitional problem in cell, oligonucleotide comprising about 10 to 40 bases, particularly about 15 to 30 bases is preferable, but it is not limited thereto. In specific, in the AIM1 gene, the 5′ end hairpin loop, 5′ end 6-base-pair repeats, 5′ end untranslated region, translation initiation codon, protein coding region, ORF translation termination codon, 3′ end untranslated region, 3′ end palindrome region, and 3′ end hairpin loop, may be selected as preferred target regions of an antisense nucleic acid, though not limited by those.

Further, the antisense nucleic acid of the invention may the nucleic acid in which the translation into a protein by hybridizing with mRNA of AIM1 or with early transcription product is inhibited, and it may as well as be the nucleic acid capable of forming a triplex by binding with the double-stranded DNA which is the gene of AIM1 and inhibiting the transcription into RNA (anti-gene).

The nucleotide molecule constituting the antisense nucleic acid may be natural DNA or RNA, but may also include various chemical modifications in order to improve the stability (chemical and/or anti-enzyme) and specific activity (affinity with RNA). For example, to prevent digestion with a hydrolase such as nuclease, etc., the phosphoric acid residue (phosphate) of each nucleotide that constitutes the antisense nucleic acid may be substituted with chemically modified phosphoric acid residues, e.g., phosphorothioate (PS), methyl phosphonate, phosphorodithionate, etc. Also, a hydroxyl group at the 2′ position of the sugar (ribose) in each nucleotide may be replaced by —OR(R represents, e.g., CH₃(2′-O-Me), CH₂CH₂OCH₃ (2′-O-MOE), CH₂CH₂NHC(NH)NH₂, CH₂CONHCH₃, CH₂CH₂CN, etc.). Further, the base part (pyrimidine, purine) may be chemically modified, for example by the introduction of a methyl group or a cationic functional group to 5-position of the pyrimidine base or the replacement of a C2 carbonyl group with thiocarbonyl, etc.

The conformation of sugar in RNA is dominant in two of C2′-endo (S-form) and C3′-endo (N-form) and exists in equilibrium of those two in single-stranded RNA, but restricted in N-form in the case of a double strand. Accordingly, in order to provide a strong binding ability to the target RNA, BNA(LNA) (Imanishi, T. et al., Chem. Commun., 1653-9, 2002; Jepsen, J. S. et al., Oligonucleotides, 14, 130-46, 2004) and ENA (Morita, K. et al., Nucleosides Nucleotides Nucleic Acids, 22, 1619-21, 2003), which are RNA derivatives of which the sugar conformation is restricted into N-form by linking 2′-oxygen with 4′-carbon, are preferably employed.

The antisense oligonucleotide of the invention can be prepared by determining a target sequence of mRNA or early transcription product on the basis of a cDNA sequence of AIM1 or genomic DNA sequence, and synthesizing its complementary sequence with the use of a commercially available DNA/RNA automatic synthesizer (Applied Biosystem company, Beckman company, etc.). In addition, the aforementioned antisense nucleic acids including various modifications can be chemically synthesized by a technique known per se.

(b) siRNA for AIM1 mRNA

In the present specification, the nucleic acid comprising a base sequence complimentary or substantially complimentary to the base sequence of AIM1 mRNA or a part of the base sequence is also defined to include so-called siRNA which is double-stranded RNA comprising oligoRNA complementary to AIM1 mRNA and a strand complementary to the oligoRNA. The phenomenon of so-called RNA interference (RNAi) in which mRNA complementary to the RNA introduced is degraded by introducing short double-stranded RNA into a cell, is known to occur in nematode, an insect, plant, etc., but after it is confirmed that the phenomenon also occurs in animal cells [Nature, 411 (6836): 494-498 (2001)], it is widely used as an another technology of ribozyme. siRNA can be appropriately designed on the basis of base sequence information of target mRNA by using a commercially available software (e.g., RNAi Designer; Invitrogen corp.). In specific, siRNAs used in EXAMPLES shown later are preferably exemplified as siRNA of the invention, but not limited by those.

The ribonucleoside molecule constituting siRNA may also be modified in the same manner as in the antisense nucleic acid described above so as to improve the stability and specific activity. However, in the case of siRNA, introducing minimal modified nucleoside on which the RISC complex can be functioned is necessary as there is a case where RNAi activity is lost by replacing all ribonucleoside molecules in natural RNA to a modified type.

siRNA can be prepared according a process comprising synthesizing a sense strand and an antisense strand of target sequence on mRNA, respectively with the DNA/RNA automatic synthesizer, modifying in a suitable annealing-buffer solution at about 90 to 95° C. for about 1 minute, and annealing at about 30 to 70° C. for about 1 to 8 hours. In addition, siRNA can also be prepared by synthesizing short hairpin RNA (shRNA) which is the precursor of siRNA and cleaving the shRNA with the use of a dicer.

(c) Nucleic Acid Capable of Producing siRNA for AIM1 mRNA

In the present specification, the nucleic acid comprising a base sequence complimentary or substantially complimentary to the base sequence of AIM1 mRNA or a part of the base sequence is also defined to include a nucleic acid designed such that aforementioned siRNA for AIM1 mRNA can be produced in vivo. Such nucleic acid can be exemplified by aforementioned shRNA, an expression vector constructed in a manner to express the shRNA, or the like. The shRNA can be prepared by designing oligoRNA comprising a base sequence in which a sense strand and an antisense strand of the target sequence on mRNA are linked by a spacer sequence of a suitable length to form a loop-structure (for example, about 15 to 25 bases) intervened between the strands, so as to synthesize by a DNA/RNA automatic synthesizer. The expression vector comprising a shRNA expression cassette can be prepared by first preparing a double-stranded DNA encoding the aforementioned shRNA and then inserting into a suitable expression vector. As the shRNA expression vector, those having Pol III promoter such as U6 or H1 can be used. In this case, shRNA transcribed in an animal cell in which the expression vector had been introduced forms a loop by itself, and then is processed by an intercalated enzyme dicer or the like to form mature siRNA.

Other preferred example of the nucleic acid comprising a base sequence complimentary or substantially complimentary to the base sequence of AIM1 mRNA or a part of the base sequence includes a ribozyme capable of specifically cleaving the internal coding region of the mRNA. ‘ribozyme’ is narrowly-defined as RNA having enzymatic activity for cleaving nucleic acid, but the present specification also includes DNA as long as there is a sequence specific enzymatic activity for cleaving nucleic acid. Ribozyme with mostly high-generality includes self-splicing RNA which can be found in infectious RNA such as viroid, a virusoid, etc., and hammer-head type or hairpin type are known. The hammer-head type exhibits enzymatic activity at about 40 bases, and it is possible to specifically cleave only the target mRNA by arranging several bases (about 10 bases in total) of both ends adjacent to the part having a hammer-head structure, so as to be complimentary to the desired cleaving site in mRNA. This type of ribozyme has a further advantage that genomic DNA is never targeted as its substrate is only RNA. When AIM1 mRNA forms itself a double-stranded structure, the target sequence can be formed into a single-strand by using hybrid ribozyme coupled with RNA motif derived from viral nucleic acid which specifically binds to RNA helicase [Proc. Natl. Acad. Sci. USA, 98 (10): 5572-5577 (2001)]. Also, in a case where ribozyme is used in the form of an expression vector having DNA which encodes the ribozyme, the ribozyme can be hybrid ribozyme further coupled with the sequence of modified tRNA so as to promote transport to cytoplasm of a transcriptional product [Nucleic Acids Res., 29 (13): 2780-2788 (2001)].

The nucleic acid comprising a base sequence complimentary or substantially complimentary to the base sequence of AIM1 mRNA or a part of the base sequence may be provided in a specialized form such as liposomes, microspheres, or may be applied to gene therapy, or may be provided in combination with attached moieties. Such attached moieties include polycations such as polylysine that act as charge neutralizers of the phosphate backbone, or hydrophobic moieties such as lipids (e.g., phospholipids, cholesterols, etc.) that enhance the interaction with cell membranes or increase uptake of the nucleic acid. Preferred examples of the lipids to be attached are cholesterols or derivatives thereof (e.g., cholesteryl chloroformate, cholic acid, etc.). These moieties may be attached to the nucleic acid at the 3′ or 5′ ends thereof and may also be attached thereto through a base, sugar, or intramolecular nucleoside linkage. Other moieties may be capping groups specifically placed at the 3′ or 5′ ends of the nucleic acid to prevent degradation by nucleases such as exonuclease, RNase, etc. Such capping groups include, but are not limited to, hydroxyl protecting groups known in the art, including glycols such as polyethylene glycol, tetraethylene glycol, and the like.

The AIM1 protein expression inhibitory activity of the nucleic acid can be determined with the use of a transformant introduced with an AIM1 gene, an AIM1 gene expression system in vivo and in vitro, or an AIM1 protein translation system in vivo and in vitro.

The substance inhibiting the expression of AIM1 protein in the invention is not limited by the aforementioned nucleic acid comprising a base sequence complimentary or substantially complimentary to the base sequence of AIM1 mRNA or a part of the base sequence, and any other substances such as low-molecular compounds or antibodies may also be employed as long as it inhibits directly or indirectly the production of AIM1 protein. Such substances can be obtained by, for example, screening methods of the invention which will be described later.

The ‘substance inhibiting the activity of AIM1 protein’ in the invention may be any of those inhibiting mechanically produced AIM1 protein to exhibit its biological activity, and examples thereof include substances inhibiting the transport of AIM1 to cell membrane, substances inhibiting the folding of AIM1 protein, substances inhibiting the function of AIM1 protein in cell membrane, and the like.

In specific, the substance inhibiting the activity of AIM1 protein can be exemplified by neutralizing antibodies against AIM1 protein. The antibody may be any of polyclonal and monoclonal antibodies. These antibodies can be manufactured in accordance with the method of manufacturing antibody or antisera, known per se. The isotype of the antibody is not particularly limited, but it is preferably IgG, IgM, or IgA, particularly preferably IgG. The antibody is not particularly subjected to limitation, as long as it has at least a complementality determining region (CDR) for specifically recognizing and binding to the target antigen; and in addition to the whole antibody molecule, the antibody may, for example, be a fragment such as Fab, Fab′, or F(ab′)₂, a genetically engineered conjugate molecule such as scFv, scFv-Fc, minibody, or diabody, or a derivative thereof modified with a molecule having protein stabilizing action, such as polyethylene glycol (PEG), or the like.

In a preferred mode of embodiment, since the antibody to the AIM1 protein is used as a pharmaceutical product targeting humans as the subject of administration thereof, the antibody (preferably a monoclonal antibody) is an antibody whose risk of showing antigenicity when administered to a human has been reduced; to be specific, the antibody is a fully human antibody, a humanized antibody, a mouse-human chimeric antibody and the like, particularly preferably a fully human antibody. A humanized antibody and a chimeric antibody can be prepared by genetic engineering technology according to the usual method. Although a fully human antibody can also be produced from the above-described human-human (or -mouse) hybridoma, it is desirable to produce it using a human antibody-producing mouse or the phage display method in order to stably supply the antibody in large amounts at low costs.

Since AIM1 variants or nucleic acids encoding thereof which exhibit dominant negative effect against AIM1 can inhibit the activity of AIM1, they can be preferably used as the substance inhibiting the activity of AIM1.

In addition, since the aptamer against AIM1 can inhibit the activity or function of A1M, it can be preferably used as the substance inhibiting the activity of AIM1. The aptamer can be acquired according to the generally known method such as SELEX (systematic evolution of ligands by exponential enrichment) method (Annual Review of Medicine, Vol. 56, 555-583, 2005). The aptamer structure can be determined using a generally known method, and on the basis of this structure, the aptamer can be produced in accordance with the generally known method.

Since there is a possibility that the AIM1 protein localizes in cell membrane, although a substance with inferior intracellular transition property such as antibody can be preferably used, there is no doubt that the low-molecular compounds obeying Lipinski's Rule can be exemplified as other preferred substance for inhibiting the activity of AIM1 protein. Such compounds can be acquired by, for example, screening methods which will be described later.

The aforementioned method (B) specifically includes the administration of a substance enhancing the expression or activity of STK17B, LOC93349, CASP8, SP110, NOD27, or RHOBTB3 (referred to as the ‘activator for STK17B, LOC93349, CASP8, SP110, NOD27, or RHOBTB3’) to mammals in need of prevention and/or treatment against cancer, preferably cancer less sensitive or insensitive to TRAIL signal activator. Accordingly, the invention also provides a preventive or remedy for cancer, by comprising an agent activating STK17B, LOC93349, CASP8, SP110, NOD27, or RHOBTB3.

The ‘substance enhancing the expression of STK17B, LOC93349, CASP8, SP110, NOD27, or RHOBTB3’ in the invention refers to a substance increasing the level of STK17B, LOC93349, CASP8, SP110, NOD27, or RHOBTB3 in vivo, and examples thereof include protein of STK17B, LOC93349, CASP8, SP110, NOD27, or RHOBTB3 itself, its partial peptide, nucleic acids encoding the protein, and the like. In addition, examples of the ‘substance enhancing the activity of STK17B, LOC93349, CASP8, SP110, NOD27, or RHOBTB3’ include agonist antibodies for STK17B, LOC93349, CASP8, SP110, NOD27, or RHOBTB3, low-molecular compounds exhibiting an agonist activity against STK17B, LOC93349, CASP8, SP110, NOD27, or RHOBTB3, and the like.

The protein of STK17B, LOC93349, CASP8, SP110, NOD27, or RHOBTB3 can be separated from cells or tissues from which the proteins are produced by publicly known protein separation and purification methods such as solvent extraction, distillation, isoelectrofocusing electrophoresis, column chromatography, liquid chromatography, reverse phase chromatography, ion exchange chromatography, affinity chromatography, recrystallization, or a combination of these techniques. Alternatively, proteins of STK17B, LOC93349, CASP8, SP110, NOD27, or RHOBTB3 or its partial peptide, can be produced according to publicly known peptide synthesis methods such as solid-phase synthesis method or liquid-phase synthesis method.

That is, the partial peptide or amino acids that can construct the protein of STK17B, LOC93349, CASP8, SP110, NOD27, or RHOBTB3 are condensed with the remaining part. Where the product contains protecting groups, these protecting groups are removed to give the desired protein. Publicly known methods for condensation and elimination of the protecting groups are described in (1) and (2) below:

### (1) M. Bodanszky & M. A. Ondetti: Peptide Synthesis, Interscience Publishers, New York (1966)

### (2) Schroeder & Luebke: The Peptide, Academic Press, New York (1965).

The protein of STK17B, LOC93349, CASP8, SP110, NOD27, or RHOBTB3 can also be produced by culturing a transformant comprising a nucleic acid encoding the protein and then separating and purifying the protein from thus obtained cultured product.

DNA encoding the protein of STK17B, LOC93349, CASP8, SP110, NOD27, or RHOBTB3 or its partial peptide can be exemplified by genomic DNA and cDNA derived from any cells or tissues of mammals or synthetic DNA. The genomic DNA and cDNA encoding the protein of STK17B, LOC93349, CASP8, SP110, NOD27, or RHOBTB3 or its partial peptide can be directly amplified by Polymerase Chain Reaction or Reverse Transcriptase-PCR with the use of genomic DNA fraction, or total RNA or mRNA fraction prepared from the above-described cells or tissues each as a template. Further, the genomic DNA and cDNA encoding the protein of STK17B, LOC93349, CASP8, SP110, NOD27, or RHOBTB3 or its partial peptide can be respectively cloned from genomic DNA library or cDNA library which are prepared by inserting the fragment of genomic DNA, or total RNA or mRNA prepared from the above-described cells or tissues into an appropriate vector, in accordance with a colony or plaque hybridization assay or PCR method. The protein of STK17B, LOC93349, CASP8, SP110, NOD27, or RHOBTB3 or its partial peptide can also be synthesized by the in vitro translation with the use of a cell-free protein translation system comprising rabbit reticulocyte lysate, wheat germ lysate, colon bacillus lysate, etc., and RNA which corresponds to DNA encoding the protein of STK17B, LOC93349, CASP8, SP110, NOD27, or RHOBTB3 or its partial peptide as a template. Alternatively, the protein or its partial peptide can also be synthesized by a cell-free transcription/translation system comprising RNA polymerase with the use of DNA encoding protein of STK17B, LOC93349, CASP8, SP110, NOD27, or RHOBTB3 or its partial peptide as a template.

The agonist antibody to the protein of STK17B, LOC93349, CASP8, SP110, NOD27 or RHOBTB3 can be acquired by the same technique as for the agonist antibody to TRAILR mentioned above. The low-molecular compound exhibiting an agonist activity against the protein of STK17B, LOC93349, CASP8, SP110, NOD27 or RHOBTB3 can be acquired by, for example, screening methods of the invention which will be described later.

According to the invention, an AIM1 inhibitor or an agent activating TK17B, LOC93349, CASP8, SP110, NOD27 or RHOBTB3 prepared into pharmaceutical preparations according to the usual practice can be used as a preventive or remedy for cancer.

For example, the composition for oral administration includes solid or liquid preparations, particularly, tablets (including dragees and film-coated tablets), pills, granules, powdery preparations, capsules (including soft capsules), syrup, emulsions, suspensions, etc. Such a composition is manufactured by publicly known methods and contains a carrier, a diluent, or excipient commonly used in the field of pharmaceutical preparations. Examples of the carrier or excipient for tablets are lactose, starch, sucrose, magnesium stearate, etc.

Examples of the composition for parenteral administration are injectable preparations, suppositories, etc. The injectable preparations may include dosage forms such as intravenous, subcutaneous, intracutaneous, and intramuscular injections, drip infusions, intraarticular injections, etc. These injectable preparations may be prepared by methods publicly known, for example, by dissolving, suspending, or emulsifying the active ingredients above in a sterile aqueous or oily liquid commonly used for injections. As the aqueous liquid for injections, there are, for example, an isotonic solution containing physiological saline, glucose or other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mols) adduct of hydrogenated castor oil)], etc. As the oily liquid, there are employed, e.g., sesame oil, soybean oil, etc., which may be used in combination with a solubilizing agent such as benzyl benzoate, benzyl alcohol, etc. The injection thus prepared is usually filled in an appropriate ampoule. The suppository used for rectal administration may be prepared by blending the compound described above with common bases for suppositories.

The aforementioned antisense nucleic acid can be prepared into pharmaceutical preparations according to the publicly known method to be administered. In addition, for example, the antisense nucleic acid alone is administered directly, or the antisense nucleic acid is inserted into an appropriate vector such as retrovirus vector, adenovirus vector, adenovirus-associated virus vector, etc., so as to be then be administered orally or parenterally to human or a mammal (e.g., rat, rabbit, sheep, swine, bovine, feline, canine, simian, etc.) in a conventional manner. The antisense nucleic acid may also be administered as it stands, or may be prepared in pharmaceutical preparations together with a physiologically acceptable carrier such as auxiliary agent to assist its uptake, which are then administered by gene gun or through a catheter such as a catheter with a hydrogel. Alternatively, the antisense nucleic acid may be prepared into an aerosol, which is topically administered into the trachea as an inhaler. Further for the purposes of improving pharmacokinetics, prolonging a half-life, and improving intracellular uptake efficiency, the antisense nucleic acid described above is prepared into pharmaceutical preparations (injectable preparations) alone or together with a carrier such as liposome, etc. and the preparations may be administered intravenously, subcutaneously, or into a joint cavity or to carcinoma lesion, etc. The aforementioned double-stranded RNA (siRNA, shRNA), ribozyme, and the variant protein or nucleic acid encoding the protein giving a dominant negative effect against the protein used in the invention mentioned above can be prepared into pharmaceutical preparations in the same manner as for the antisense nucleic acid, which can be then administered.

The aforementioned antibody, aptamer, or the like can be administered as it stands or as an appropriate pharmaceutical composition. The pharmaceutical composition which can be used for the administration above is those containing a pharmacologically acceptable carrier, a diluent or excipient with the antibody or a salt thereof. Such composition is provided in the form of pharmaceutical preparations suitable for oral or parenteral administration (e.g., intravenous injection), preferably as inhalant.

The aforementioned compositions may contain other active ingredients (e.g., other anticancer agents, etc.) within the scope of not causing the undesired interaction due to the blending with the active ingredient. In addition the composition may be used in combination with other active ingredients (e.g., other anticancer agents, etc.), and a preferred example of the other active ingredients include an TRAIL signal activator. Since the AIM1 inhibitor and the activator for STK17B, LOC93349, CASP8, SP110, NOD27, or RHOBTB3 increase the sensitivity to TRAIL signal activator in patients, they can be used in combination to significantly increase the drug efficiency of TRAIL signal activator. In the case of using a TRAIL signal activator as the other active ingredient, same dosage as before is applied.

### 4. Screening of Regulator for Trail Signal Activator Sensitive-Related Factor

The present invention also provides a method of screening a compound or a salt thereof having an anticancer effect on the basis of (A) inhibition activity for AIM1 expression or activity or (B) activity for activating STK17B, LOC93349, CASP8, SP110, NOD27, or RHOBTB3, as a reference.

In the case of screening the compound or a salt thereof which inhibits the activity of AIM1 protein or enhances the activity of the protein of STK17B, LOC93349, CASP8, SP110, NOD27, or RHOBTB3, the screening method includes culturing cells capable of producing the AIM1 protein or the protein of STK17B, LOC93349, CASP8, SP110, NOD27, or RHOBTB3 under two conditions of with and without a test compound, and comparing activities of the proteins in two different conditions.

The cell capable of producing the AIM1 protein or the protein of STK17B, LOC93349, CASP8, SP110, NOD27, or RHOBTB3 used in the above screening method is not particularly limited as long as it is a human or other mammalian cell, or a biological specimen containing thereof (e.g., blood, tissue, organ, etc.), and preferred examples thereof include TRAIL signal activator-resistance cancers and the like. In the case of non-human animal-derived blood, tissue, organ, or the like, it may be isolated from an organism to be cultured, or a test compound may be administered to an organism per se and its biological specimen may be isolated after a certain period of time.

The cells capable of producing the AIM1 protein or the protein of STK17B, LOC93349, CASP8, SP110, NOD27, or RHOBTB3 or its partial peptide can be exemplified by various transformants prepared by well-known genetic engineering techniques. As the host, for example, a H4IIE C3 cell, a HepG2 cell, a HEK293 cell, a COST cell, a CHO cell, or the like can be preferably employed.

To be specific, the preparation can be carried out by ligating DNA (that is, DNA comprising a base sequence represented by SEQ ID NO: 1, 3, 5, 7, 9, 11, or 13, or a base sequence hybridizable to the base sequence represented by SEQ ID NO: 1, 3, 5, 7, 9, 11 under high stringent conditions and comprising the sequence encoding a polypeptide having the same activity as the protein comprising an amino acid sequence represented by SEQ ID NO: 2, 4, 6, 8, 10, 12, or 14) which encodes the AIM1 protein or the protein of STK17B, LOC93349, CASP8, SP110, NOD27, or RHOBTB3 or its partial peptide downstream of a promoter in an appropriate expression vector, and transfecting in a host animal cell.

DNAs that encode the AIM1 protein or the protein of STK17B, LOC93349, CASP8, SP110, NOD27, or RHOBTB3 or its partial peptide, can be cloned, for example, by hybridization or PCR technique from cDNA library or cDNA derived from cell/tissue which produce the AIM1 protein or the protein of STK17B, LOC93349, CASP8, SP110, NOD27, or RHOBTB3 with the use of appropriate oligonucleotides synthesized as a probe or primers on the bases of the base sequence represented by SEQ ID NO: 1, 3, 5, 7, 9, 11, or 13. The hybridization can be carried out, for example, according to the method described in Molecular Cloning, 2nd edition (J. Sambrook et al., Cold Spring Harbor Lab. Press, 1989). Where the hybridization is carried out using commercially available library, the procedures may be conducted in accordance with the protocol described in the attached instructions.

Substitution of the base sequence of DNA can be effected by publicly known methods such as the ODA-LA PCR method, the Gapped duplex method, the Kunkel method, etc., with the use of a publicly known kit such as Mutan™-super Express Km (Takara Bio) or Mutan™-K (Takara Bio), etc.

The cloned DNA can be used as it is, depending on the purpose or, if desired, after digestion with a restriction enzyme or after addition of a linker thereto. The DNA may contain ATG as a translation initiation codon at the 5′ end thereof and TAA, TGA, or TAG as a translation termination codon at the 3′ end thereof. These translation initiation and termination codons may also be added by using an appropriate synthetic DNA adapter.

Examples of the expression vector include animal cell expression plasmids (e.g., pA1-11, pXT1, pRc/CMV, pRc/RSV, pcDNA I/Neo, etc.); bacteriophages such as λ phage; animal virus vectors such as retrovirus, vaccinia virus, adenovirus; and the like. The promoter may be any promoter if it matches well with a host to be used for gene expression. Examples of the promoter include SRα promoter, SV40 promoter, LTR promoter, CMV (cytomegalovirus) promoter, RSV (Rous sarcoma virus) promoter, MoMuLV (Moloney murine leukemia virus) LTR, HSV-TK (simplex virus-thymidine kinase) promoter, and the like. Among them, it is preferable to use CMV promoter, SRα promoter, or the like.

In addition to the foregoing examples, the expression vector may further optionally contain an enhancer, a splicing signal, a poly A addition signal, a selection marker, SV40 replication origin (hereinafter sometimes abbreviated as SV40ori), etc. Examples of the selection marker include dihydrofolate reductase gene (hereinafter sometimes abbreviated as dhfr, methotrexate (MTX) resistance), ampicillin resistant gene (hereinafter sometimes abbreviated as amp^{r}), neomycin resistant gene (hereinafter sometimes abbreviated as Neo^{r}, G418 resistance), etc. In particular, when dhfr gene is used as the selection marker using dhfr gene-deficient Chinese hamster cells, the target gene can be selected on a thymidine free medium.

The host is being transformed with the expression vector comprising DNA which encodes the AIM1 protein or the protein of STK17B, LOC93349, CASP8, SP110, NOD27, or RHOBTB3, thereby producing the cell expressing the AIM1 protein or the protein of STK17B, LOC93349, CASP8, SP110, NOD27, or RHOBTB3.

Examples of animal cells as the host include HepG2 cell, HEK293 cell, HeLa cell, human FL cell, simian COS-7 cell, simian Vero cell, Chinese hamster ovary cell (hereinafter referred to as CHO cell), dhfr gene-deficient CHO cell (hereinafter simply referred to as CHO (dhfr⁻)) cell), mouse L cell, mouse AtT-20 cell, mouse myeloma cell, rat H4IIE-C3 cell, rat GH3 cell, and the like.

The transformation can be carried out according to, for example, PEG method, electroporation method, microinjection method, lipofection method, or the like. For example, the method described in Saibo Kogaku (Cell Engineering), extra issue 8, Shin Saibo Kogaku Jikken Protocol (New Cell Engineering Experimental Protocol), 263-267 (1995) (published by Shujunsha), or Virology, VOL. 52, 456 (1973) can be employed.

The thus obtained transformed cells or mammalian cells capable of producing native AIM1 protein or the protein of STK17B, LOC93349, CASP8, SP110, NOD27, or RHOBTB3, or tissues/organs comprising the cell can be cultured in, for example, Minimum Essential Medium (MEM) containing about 5 to 20% fetal bovine serum [Science, 122, 501 (1952)], Dulbecco's Modified Eagle's Medium (DMEM) [Virology, 8, 396 (1959)], RPMI 1640 medium [The Journal of the American Medical Association, 199, 519 (1967)], 199 medium [Proceeding of the Society for the Biological Medicine, 73, 1 (1950)], etc. Preferably, pH of the medium is adjusted to about 6 to about 8. The transformant is usually cultivated at about 30° C. to 40° C. and, if necessary, the culture can be aerated or agitated.

Examples of the test compound include peptides, proteins, antibody, non-peptide compounds, synthetic compounds, fermentation products, cell extracts, plant extracts, animal tissue extracts, plasma, and the like. These may be either a novel substance or well-known substance. The test compound may form salts, and examples of salts of the test compound include physiologically acceptable metal salts, ammonium salts, salts with organic bases, salts with inorganic acids, salts with organic acid, salts with basic or acidic amino acids, and the like. Suitable examples of the metal salts include alkali metal salts such as sodium salt, and potassium salt; alkali earth metal salts such as calcium salt, magnesium salt, and barium salt; and aluminum salt, and the like. Suitable examples of the salts with organic bases include salts with trimethylamine, triethylamine, pyridine, picoline, 2,6-lutidine, ethanolamine, diethanol amine, triethanol amine, cyclohexylamine, dicyclohexylamine, N,N′-dibenzylethylenediamine, and the like. Suitable examples of the salts with inorganic acids include salts with hydrochloric acid, hydrobromic acid, nitric acid, sulfuric acid, phosphoric acid, and the like. Suitable examples of the salts with organic acids include formic acid, acetic acid, trifluoroacetic acid, propionic acid, phthalic acid, fumaric acid, oxalic acid, tartaric acid, maleic acid, citric acid, succinic acid, malic acid, methanesulfonic acid, benzoic acid, benzenesulfonic acid, p-toluenesulfonic acid, and the like. Suitable examples of the salts with basic amino acids include salts with arginine, lysine, ornithine, and the like. Suitable examples of the salts with acidic amino acids include salts with asparagic acid, glutamic acid, and the like.

The test compound can be contacted with the above-described cell by for example, adding the test compound into the above-described medium and each buffer (e.g., HEPES buffer, phosphate buffer, phosphate buffered saline, tris-hydrochloride buffer, borate buffer, and acetate buffer, etc.), incubating cells for a certain period of time. The concentration of the test compound to be added differs depending on the kind of the compound (solubility, toxicity, and the like), but appropriately selected for example, in the range of approximately 0.1 nM to 100 nM. The incubation time may be exemplified by approximately 10 minutes to 24 hours.

Where cells producing AIM1 protein or STK17B, LOC93349, CASP8, SP110, NOD27 or RHOBTB3 protein, are provided in the form of individual non-human mammal, conditions of the mammalian is not particularly limited. The conditions for bleeding animal to be used are not particularly limited, but those bred under environment of higher than SPF grade are preferable. The test compound can be brought into contacted with the cell by administrating the test compound to the individual mammal. The administration route is not particularly limited, but may employ, for example, intravenous administration, intraarterial administration, subcutaneous administration, intradermal administration, intraperitoneal administration, oral administration, intraairway administration, rectal administration, and the like. The dose of the test compound to be administered is not particularly limited, but the test compound may be administrated for example, in a dose of about 0.5 to 20 mg/kg, 1 to 5 times/day, preferably about 1 to 3 times/day, for 1 to 14 day(s).

The measurement of the activities of AIM1 protein or STK17B, LOC93349, CASP8, SP110, NOD27 or RHOBTB3 protein in the screening method, can be carried out in accordance with the same method described in the each protein. The test sample for the activity measurement may be exemplified by a extract of culture, where cells producing AIM1 protein or STK17B, LOC93349, CASP8, SP110, NOD27 or RHOBTB3 protein, are provided in the form of cell culture, tissue culture, or organ culture; and a extracts of cells, tissues, or organs, separated from individual, or a homogenate of segment of these tissues, where the cells are provided in the form of individual non-human mammal containing the cells.

For example, in the above-described screening method, when the activity of AIM1 protein is inhibited, in the presence of a test compound, by at least about 20%, preferably at least about 30%, and more preferably at least about 50%, compared to the activity in the absence of the test compound, the test compound or a salt thereof can be selected as an inhibitor of AIM1 protein, and therefore, as a candidate for a substance having a TRAIL signal activator sensitivity improving effect and an anticancer effect. Also, when the activity of STK17B, LOC93349, CASP8, SP110, NOD27 or RHOBTB3 protein is enhanced, in the presence of a test compound, by at least about 20%, preferably at least 30%, and more preferably at least about 50%, compared to the activity in the absence of the test compound, the test compound or a salt thereof can be selected as an activator of STK17B, LOC93349, CASP8, SP110, NOD27 or RHOBTB3 protein, and therefore, as a candidate for a substance having a TRAIL signal activator sensitivity improving effect and an anticancer effect.

The invention also provides a method of screening a compound having an anticancer effect, or a salt thereof, characterized in that the expression of AIM1 protein, or STK17B, LOC93349, CASP8, SP110, NOD27 or RHOBTB3 protein in cells having an ability to produce the protein (gene) in the presence of a test compound, and the expression in the absence of the test compound are compared. The cells used in the present method, the type of the test compound, the mode of contacting the test compound with cells, and the like are the same as in the above-described method of using the activity of the AIM1 protein, or STK17B, LOC93349, CASP8, SP110, NOD27 or RHOBTB3 protein as an index.

The level of expression of AIM1 protein, or STK17B, LOC93349, CASP8, SP110, NOD27 or RHOBTB3 protein can be measured at the RNA levels, by detecting the mRNA of AIM1 or STK17B, LOC93349, CASP8, SP110, NOD27 or RHOBTB3 using a nucleic acid which can be hybridized with the DNA encoding the AIM1 protein, or the STK17B, LOC93349, CASP8, SP110, NOD27 or RHOBTB3 protein under stringent conditions, namely, nucleic acids which can be hybridized with base sequences represented by SEQ ID NO:1, 3, 5, 7, 9, 11 or 13, and base sequences complementary thereto, under stringent conditions (hereinafter, may be referred to as “nucleic acid for detection of the invention”). Alternatively, the level of expression can also be measured at the protein level, by detecting the AIM1 protein, or the STK17B, LOC93349, CASP8, SP110, NOD27 or RHOBTB3 protein using antibodies to these proteins (hereinafter, may be referred to as “antibody for detection of the invention”).

Accordingly, the present invention more specifically provides:

(a) a method of screening the compound having anticancer effect or a salt thereof, in which the method is characterized in that a cell capable of producing AIM1 protein or STK17B, LOC93349, CASP8, SP110, NOD27 or RHOBTB3 protein, is cultured in the presence of and absence of test compound, and the amount of mRNA encoding the protein obtained under two different conditions are measured and compared using an nucleic acid for detection; and

(b) a method of screening the compound having anticancer effect or a salt thereof, in which the method is characterized in that a cell capable of producing AIM1 protein or STK17B, LOC93349, CASP8, SP110, NOD27 or RHOBTB3 protein, is cultured in the presence of and absence of test compound, and the amount of the protein obtained under two different conditions are measured and compared using an antibody for detection of the invention.

For example, a quantification of mRNA or protein of AIM1, or, STK17B, LOC93349, CASP8, SP110, NOD27 or RHOBTB3 can be specifically carried out as the following:

(i) normal or abnormal model (for example, gallbladder cancer) non-human mammalian (for example, mouse, rat, rabbit, sheep, swine, bovine, feline, canine, simian, etc.) is administered with a medicine, and after a given period of time, blood, or particular tissues (for example, a cancer tissue) are obtained.

mRNA of AIM1, or STK17B, LOC93349, CASP8, SP110, NOD27 or RHOBTB3 contained in the obtained cell can be, for example, extracted from cells according to normally used method and quantified by using methods such as RT-PCR. The mRNA can also be quantified by Northern blot analysis known per se. On the other hand, AIM1, or STK17B, LOC93349, CASP8, SP110, NOD27 or RHOBTB3 protein can be quantified by a Western blot analysis or by a various immunoassay method described below.

(ii) A transformant into which a polynucleotide encoding the AIM1, or STK17B, LOC93349, CASP8, SP110, NOD27 or RHOBTB3 protein is introduced is prepared according to above-mentioned method, and AIM1, STK17B, LOC93349, CASP8, SP110, NOD27 or RHOBTB3 protein, or mRNA encoding the same contained in the transformant can be quantified and analyzed in the same manner as in the (i) above.

A screening of a substance which modifies the expression level of AIM1, or STK17B, LOC93349, CASP8, SP110, NOD27 or RHOBTB3 can be carried out by:

(i) administering a test compound to a normal or disease model non-human mammal at a given time before (before 30 minutes to before 24 hours, preferably before 30 minutes to before 12 hours, more preferably before 1 hour to before 6 hours) or after (after 30 minutes to after 3 days, preferably after 1 hour to after 2 days, more preferably after 1 hour to after 24 hours) a drug is given, or simultaneously with the drug etc., and quantifying and analyzing the amount of mRNA in the cell isolated from an animal, which encodes AIM1, or STK17B, LOC93349, CASP8, SP110, NOD27 or RHOBTB3 included in the cell, or the amount of protein of AIM1, or STK17B, LOC93349, CASP8, SP110, NOD27 or RHOBTB3, after an elapse of a given time from administration (after 30 minutes to after 3 days, preferably after 1 hour to after 2 days, more preferably after 1 hour to after 24 hours), and can also be preformed by:
(ii) adding a test compound to a medium or buffer when cultivating a transformant according to a conventional method, and, after incubation for a given period of time (1 to 7 days after, preferably 1 to 3 days after, more preferably 2 to 3 days after), and quantifying and analyzing the amount of mRNA in the transformant, which encodes AIM1, or STK17B, LOC93349, CASP8, SP110, NOD27 or RHOBTB3 included in the cell, or the amount of protein of AIM1, or STK17B, LOC93349, CASP8, SP110, NOD27 or RHOBTB3.

Quantification for the protein of the present invention in the screening method (b) above specifically includes:

(i) a method comprising competitively reacting the antibody of the present invention, a sample fluid, and a labeled form of the protein of AIM1, or STK17B, LOC93349, CASP8, SP110, NOD27 or RHOBTB3, and detecting the labeled protein that binds to the antibody, thereby to quantify the protein of AIM1, or STK17B, LOC93349, CASP8, SP110, NOD27 or RHOBTB3 in the sample fluid; and

(ii) a method comprising simultaneously or continuously reacting a sample fluid, the immobilized antibody of the present invention on a carrier, and a labeled form of another antibody of the present invention, and measuring the amount (activity) of the label on the immobilization carrier, thereby to quantify the protein of AIM1, or STK17B, LOC93349, CASP8, SP110, NOD27 or RHOBTB3 invention in the sample fluid.

In the quantification method of (ii) above, two species of antibodies are desirably the ones that each recognizes the different part in the protein of AIM1, or STK17B, LOC93349, CASP8, SP110, NOD27 or RHOBTB3. For example, when one antibody recognizes the N-terminal region of two pieces of the proteins, another antibody recognizing the C-terminal region is used.

Examples of labeling agents, which are employed for the measuring methods using labeling substances, include radioisotopes, enzymes, fluorescent substances, luminescent substances, and the like. Examples of radioisotopes are [¹²⁵I], [¹³¹I], [³H], [¹⁴C], [³²P], [³³P], [³⁵S], etc. Preferred examples of the enzymes are those that are stable and have a higher specific activity, which include (β-galactosidase, (β-glucosidase, alkaline phosphatase, peroxidase, malate dehydrogenase, etc. Examples of the fluorescent substances include fluorescamine, fluorescein isothiocyanate, cyanine fluorochrome, etc. Examples of the luminescent substances are luminol, a luminol derivative, luciferin, lucigenin, etc. Furthermore, a biotin-(strepto)avidin system may be used as well for binding an antibody or antigen to a labeling agent.

The quantification method of the protein of AIM1, STK17B, LOC93349, CASP8, SP110, NOD27, or RHOBTB3, using the antibody for detection of the invention is not particularly limited. Any quantification method may be used, so long as the amount of an antibody, antigen, or antibody-antigen complex corresponding to the amount of antigen in a test sample fluid can be detected by chemical or physical means and the amount of the antigen can be calculated from a standard curve prepared from standard solutions containing known amounts of the antigen. For such an assay method, for example, nephrometry, the competitive method, the immunometric method, the sandwich method, etc. are suitably used, and in terms of sensitivity and specificity, it is preferred to use the sandwich method described later.

In the immobilization of antigens or antibodies, physical adsorption may be used. Alternatively, chemical binding that is commonly used for immobilization/stabilization of proteins, enzymes, etc. may be used as well. Examples of the carrier include insoluble polysaccharides such as agarose, dextran, cellulose, etc.; synthetic resins such as polystyrene, polyacrylamide, silicone, etc.; or glass; and the like.

In the sandwich method, the immobilized antibody for detection of the invention is reacted with a test fluid (primary reaction), then with a labeled form of another antibody for detection of the invention (secondary reaction), and the amount or activity of the labeling agent on the immobilizing carrier is measured, whereby the amount of the protein of AIM1, STK17B, LOC93349, CASP8, SP110, NOD27, or RHOBTB3 used in the test fluid can be quantified. The order of the primary and secondary reactions may be reversed, or these reactions may be performed simultaneously or at staggered times. The methods of labeling and immobilization can be performed by the methods described above. In the immunoassay by the sandwich method, the antibody used for immobilized or labeled antibodies is not necessarily one species, and a mixture of two or more species of antibody may be used to increase the measurement sensitivity.

The antibody of the invention can also be used in measuring system other than the sandwich method such as in the competitive method, the immunometric method, nephrometry, etc.

In the competitive assay, the protein of AIM1, STK17B, LOC93349, CASP8, SP110, NOD27, or RHOBTB3 and a labeled form of the protein of AIM1, STK17B, LOC93349, CASP8, SP110, NOD27, or RHOBTB3 in a simple fluid are reacted competitively against an antibody, an unreacted labeled antigen (F) is separated from an antibody-bound labeled antigen (B) (B/F separation), and the labeled amount of B or F is determined, thereby to quantify the protein of AIM1, STK17B, LOC93349, CASP8, SP110, NOD27, or RHOBTB3 in the sample fluid. The present reaction method includes a liquid phase method in which the B/F separation is carried out by using a soluble antibody as an antibody and using a secondary antibody against polyethylene glycol or against the antibody (primary antibody); and a solid phase method in which a solid-phased antibody is used as a primary antibody (direct method) or a soluble antibody is used as a primary antibody and a solid-phased antibody is used as a secondary antibody (indirect method).

In the immunometric assay, the protein of AIM1, STK17B, LOC93349, CASP8, SP110, NOD27, or RHOBTB3 in a sample fluid and a solid phase protein of AIM1, STK17B, LOC93349, CASP8, SP110, NOD27, or RHOBTB3 are competitively reacted with a given amount of a labeled form of the antibody followed by separating the solid phase from the liquid phase; or the protein of AIM1, STK17B, LOC93349, CASP8, SP110, NOD27, or RHOBTB3 in a sample fluid and an excess amount of labeled form of the antibody are reacted, then a solid phase protein of AIM1, STK17B, LOC93349, CASP8, SP110, NOD27, or RHOBTB3 is added to bind an unreacted labeled form of the antibody to the solid phase and the solid phase is then separated from the liquid phase. Next, the labeled amount in any of the phases is measured to determine the antigen level in the test fluid.

In the nephrometry, the amount of insoluble sediment, which is produced as a result of the antigen-antibody reaction in a gel or in a solution, is measured. Even when the amount of the protein of AIM1, STK17B, LOC93349, CASP8, SP110, NOD27, or RHOBTB3 in a test fluid is small and only a small amount of the sediment is obtained, a laser nephrometry utilizing laser scattering can be suitably used.

For applying each of these immunological methods to the quantification method of the present invention, any particular conditions or procedures are not required. The measurement system for the protein of AIM1, STK17B, LOC93349, CASP8, SP110, NOD27, or RHOBTB3 can be constructed considering the technical skill of the person skilled in the art under usual conditions and procedures for each method. For the details of these general technical means, the aforementioned reviews and texts can be used as a reference.

In the above manner, the amount of the protein of AIM1, STK17B, LOC93349, CASP8, SP110, NOD27, or RHOBTB3 in a cell is quantified with a good sensitivity by using the antibody for detection of the invention.

For example, in the aforementioned screening methods of (a) and (b), when the expression (the amount of mRNA or protein) of AIM1 in the presence of a test compound is inhibited by at least about 20%, preferably at least 30%, and more preferably at least about 50%, as compared to the case where expression is made in the absence of a test product, the test compound or a salt thereof can be selected as an inhibitory agent for AIM1, such to be a substance having an effect on improving sensitivity to TRAIL signal activator and an anticancer effect. When the expression (the amount of mRNA or protein) of STK17B, LOC93349, CASP8, SP110, NOD27 or RHOBTB3 is increased, in the presence of a test compound, by at least about 20%, preferably at least about 30%, and more preferably at least about 50%, compared to the case in the absence of the test compound, the test compound or a salt thereof can be selected as an activator of STK17B, LOC93349, CASP8, SP110, NOD27 or RHOBTB3, and therefore, a candidate for a substance having a TRAIL signal activator sensitivity improving effect and an anticancer effect.

In the present specification, where bases, amino acids, etc. are denoted by their codes, they are based on conventional codes in accordance with the IUPAC-IUB Commission on Biochemical Nomenclature or by the common codes in the art, examples of which are shown below. For amino acids that may have the optical isomer, L form is presented unless otherwise indicated.

DNA: deoxyribonucleic acid

cDNA: complementary deoxyribonucleic acid

A: adenine

T: thymine

G: guanine

C: cytosine

RNA: ribonucleic acid

mRNA: messenger ribonucleic acid

dATP: deoxyadenosine triphosphate

dTTP: deoxythymidine triphosphate

dGTP: deoxyguanosine triphosphate

dCTP: deoxycytidine triphosphate

ATP: adenosine triphosphate

EDTA: ethylenediaminetetraacetic acid

SDS: sodium dodecyl sulfate

Gly: glycine

Ala: alanine

Val: valine

Leu: leucine

Ile: isoleucine

Ser: serine

Thr: threonine

Cys: cysteine

Met: methionine

Glu: glutamic acid

Asp: asparagic acid

Lys: lysine

Arg: arginine

His: histidine

Phe: phenylalanine

Tyr: tyrosine

Trp: tryptophan

Pro: proline

Asn: asparagine

Gln: glutamine

pGlu: pyroglutamic acid

Sec: selenocysteine

The sequence identification numbers in the sequence listing of the specification indicate the following sequences.

### [SEQ ID NO: 1]

This shows the base sequence of cDNA encoding AIM1.

### [SEQ ID NO: 2]

This shows the amino acid sequence of AIM1.

### [SEQ ID NO: 3]

This shows the base sequence of cDNA encoding STK17B.

### [SEQ ID NO: 4]

This shows the amino acid sequence of STK17B.

### [SEQ ID NO: 5]

This shows the base sequence of cDNA encoding LOC93349.

### [SEQ ID NO: 6]

This shows the amino acid sequence of LOC93349.

### [SEQ ID NO: 7]

This shows the base sequence of cDNA encoding CASP8.

### [SEQ ID NO: 8]

This shows the amino acid sequence of CASP8.

### [SEQ ID NO: 9]

This shows the base sequence of cDNA encoding SP110.

### [SEQ ID NO: 10]

This shows the amino acid sequence of SP110.

### [SEQ ID NO: 11]

This shows the base sequence of cDNA encoding NOD27.

### [SEQ ID NO: 12]

This shows the amino acid sequence of NOD27.

### [SEQ ID NO: 13]

This shows the base sequence of cDNA encoding RHOBTB3.

### [SEQ ID NO: 14]

This shows the amino acid sequence of RHOBTB3.

### [SEQ ID NO: 15]

This shows the base sequence of cDNA encoding TRAIL.

### [SEQ ID NO: 16]

This shows the amino acid sequence of TRAIL.

### [SEQ ID NO: 17]

This shows the base sequence of cDNA encoding TRAILR1.

### [SEQ ID NO: 18]

This shows the amino acid sequence of TRAILR1.

### [SEQ ID NO: 19]

This shows the base sequence of cDNA encoding TRAILR2 (Long form).

### [SEQ ID NO: 20]

This shows the amino acid sequence of TRAILR2 (Long form).

### [SEQ ID NO: 21]

This shows the base sequence of cDNA encoding TRAILR2 (Short form).

### [SEQ ID NO: 22]

This shows the amino acid sequence of TRAILR2 (Short form).

### [SEQ ID NO: 23]

This shows the base sequence of cDNA encoding TRAILR1 agonist antibody.

[SEQ ID NO: 24]

This shows the amino acid sequence of TRAILR1 agonist antibody.

The cell line TRAIL(NSO) 14G03 No. 39 P:14 Jul. 2, 2001 containing a cDNA encoding a TRAILR1 agonist antibody having the amino acid sequence shown by SEQ ID NO: 24 has been deposited since Jul. 30, 2001 at the American Type Culture Collection (ATCC) under deposition No. PTA-3570. ATCC is located at 10801 University Boulevard, Manassas, Va. 20110-2209, USA. The deposit at the ATCC is under the International Budapest Treaty for deposits of microorganisms that meets patent office requirements.

### EXAMPLES

Hereinafter, the present invention will be described in more detail with reference to EXAMPLES, but these are only the examples and not intended to limit the scope of the invention in any way.

### Example 1

### Investigation on Suppression of Proliferation of TRAILR1 Agonist Antibody in Human Cancer Cell Line

A TRAILR1 agonist antibody activates TRAIL signal, thus inducing apoptosis in cells. The sensitivity of TRAILR1 agonist antibody was investigated using the following cell lines. Human colon cancer cell lines COLO205, HCT15, DLD1, COLO201, COLO320DM; human lung cancer cell lines NCI-H460, NCI-H2122, NCI-H358, PC14, NCI-H1703, NCI-H23; human breast cancer cell lines Zr75-1, BT474; and gastric cancer cell line SNU-668, were suspended in 10 ml of RPMI1640 medium (Sigma) containing 10% fetal calf serum (JRH) and 50 μg/ml of penicillin/streptomycin (Invitrogen), and under a 5% carbon dioxide gas stream, the suspensions were cultured at 37° C. using a 10-cm culture plate. Likewise, human colon cancer cell line HCT116, human lung cancer cell line A549, and human breast cancer cell lines MCF7 and SKBr3 were suspended in 10 ml of Dulbecco's modified Eagle's medium (Sigma) containing 10% fetal calf serum (JRH) and 50 μg/ml of penicillin/streptomycin (Invitrogen), and under a 5% carbon dioxide gas stream, the suspensions were cultured at 37° C. using a 10-cm culture plate. Also, human colon cancer cell lines SW480, SW48, SW620; and human breast cancer cell lines MDA-MB-4355, MDA-MB-231, MDA-MB-436, MDA-MB-175VII and MDA-MB-468 were suspended in Leibovitz's L-15 medium (Invitrogen) containing 10% fetal calf serum (JRH) and 50 μg/ml of penicillin/streptomycin (Invitrogen), and under a natural air stream, the suspensions were cultured at 37° C. using a 10-cm culture plate. Human colon cancer cell lines WiDr and LS180 were suspended in 10 ml of Minimum Essential Medium (MEM, Invitrogen) containing 10% fetal calf serum (JRH) and 50 μg/ml of penicillin/streptomycin (Invitrogen), and under a 5% carbon dioxide gas stream, the suspensions were cultured at 37° C. using a 10-cm culture plate. For the 28 cell lines in total, the respective cells were harvested using trypsin/ETDA (Invitrogen), suspended in the respective culture solution to a concentration of 5.0×10³ cells/50 μl, and inoculated to each well of a 96-well plate in an amount of 50 μl each. At this time, the cell lines cultured in Leibovitz's L-15 medium were transferred to Dulbecco's modified Eagle's medium, and all were cultured at 37° C. under a 5% carbon dioxide stream. After 24 hours, 50 μl of TRAILR1 agonist antibody (scFv antibody encoded by the base sequence represented by SEQ ID NO:23) was added at various concentrations, obtained by dilution with the same culture solutions as those used for the respective cell cultures. After 48 hours, 10 μl of cell counting kit-8 (Dojin) was added, and after 3 hours, the absorbance at 450 nm was measured as an index to the number of live cells. The absorbance obtained under no addition of the TRAILR1 agonist antibody was taken as 100%, and the IC₅₀ was calculated. An IC₅₀ of 100 nM or less was defined to be TRAILR1 agonist antibody sensitive. The suppliers of these human cell lines, and the resistance and sensitivity of the cell lines are summarized in Table 1. As such, the TRAIL signal sensitivity varied greatly according to the cell lines.

TABLE 1
Purchased Place and Sensitivity of Learning Cell lines

| Kind of | | | IC50 | |
|---|---|---|---|---|
| Cancer | Cell Lines | Purchased Place | (nM) | Sensitivity* |
| Colon | COLO205 | DAINIPPON SUMITOMO PHARMA | 1.5 | S |
| cancer | HCT15 | American Type Culture | 2.9 | S |
| | | Collection | | |
| | HCT116 | American Type Culture | 3.5 | S |
| | | Collection | | |
| | DLD1 | American Type Culture | 8.3 | S |
| | | Collection | | |
| | SW480 | American Type Culture | 0.9 | S |
| | | Collection | | |
| | COLO201 | DAINIPPON SUMITOMO PHARMA | 29.9 | S |
| | SW48 | American Type Culture | 13.4 | S |
| | | Collection | | |
| | COLO320DM | DAINIPPON SUMITOMO PHARMA | >238 | R |
| | SW620 | American Type Culture | >238 | R |
| | | Collection | | |
| | WiDr | American Type Culture | >238 | R |
| | | Collection | | |
| | LS180 | DAINIPPON SUMITOMO PHARMA | >238 | R |
| Lung | NCI-H2122 | American Type Culture | 2.1 | S |
| Cancer | | Collection | | |
| | NCI-H460 | American Type Culture | 0.29 | S |
| | | Collection | | |
| | NCI-H358 | American Type Culture | 4.2 | S |
| | | Collection | | |
| | PC-14 | DAINIPPON SUMITOMO PHARMA | 26.5 | S |
| | NCI-H23 | American Type Culture | >238 | R |
| | | Collection | | |
| | A549 | American Type Culture | >238 | R |
| | | Collection | | |
| | NCI-H1703 | American Type Culture | >238 | R |
| | | Collection | | |
| Breast | MDA-MB-231 | American Type Culture | 28 | S |
| Cancer | | Collection | | |
| | MDA-MB-435S | American Type Culture | 8 | S |
| | | Collection | | |
| | MDA-MB-436 | American Type Culture | 2.1 | S |
| | | Collection | | |
| | MDA-MB- | American Type Culture | 19 | S |
| | 175VII | Collection | | |
| | MCF7 | American Type Culture | >238 | R |
| | | Collection | | |
| | BT474 | American Type Culture | >238 | R |
| | | Collection | | |
| | SKBr3 | American Type Culture | >238 | R |
| | | Collection | | |
| | MDA-MB-468 | American Type Culture | >238 | R |
| | | Collection | | |
| | Zr75-1 | American Type Culture | >238 | R |
| | | Collection | | |
| Stomach | SNU-668 | Korean Cell line Bank | 0.69 | S |
| Cancer | | | | |

SENSITIVITY*: those with IC50 value of 100 nM or less are ranked as sensitive strain (S) and those with equal or greater than that value are ranked as resistant strain (R)

### Example 2

### Examination on TRAIL Sensitive Testing Marker

### Exhaustive Analysis of Gene Expression

The following experiment was carried out in order to select a gene to be used for distinguishing the predicted effect against TRAIL-induced apoptosis. Total RNA was extracted from cancer cell lines, all of which expresses TRAILR1 on its cell membrane, of all 6 species including three human cancer cell lines each showing sensitivity (COLO205, NCI-H2122, and SNU668) and three human cancer cell lines each showing insensitivity (COLO320DM, SW620, and NCI-H23) to TRAILR1 agonist antibody which are described in Example 1, with the use of RNeasy Mini Kit (QIAGEN). The analysis of gene expression was carried out using the total RNA as a substance with the use of oligonucleotide microarray (Human Genome U133 plus 2.0; Affymetrix). The experimental procedure was followed as instructed in Expression analysis technical manual from Affymetrix. The experimental results thus obtained were analyzed using GeneSpring (Agilent Technology).

From genes decided as that detection call of each gene is Present on at least one line, genes approved to show expression fluctuation in sensitive strain and resistance strain were selected. The P-value related to the presence of expression fluctuation between two groups can be calculated for each gene by Student's t-test and Signal to Noise, (((mean value 1)−(mean value 2))/((SD1)+(SD2))), and as a result, the number of genes showing expression fluctuation of which the P-values from both the two assays are less than 0.005 was 272. With the use of one or plural number of these genes, it was thought as that the sensitivity of TRAIL-induced apoptosis can be predicted by subjecting the measurement on the expression level, the protein amount, or the activity.

### Example 3

### Practice of Quantitative PCR of Selector Gene

With regard to the genes further selected from genes showing fluctuation in expression described in Example 2 with reference to the gene expression data base provided from Gene Logic Inc., the gene expression level in cell lines were measured by subjecting a quantitative PCR reaction using FAM-labeled TaqMan probe to 28 strains of the human cancer cell line described in Example 1 (Table 1). According to the reaction, total RNA was prepared from the above-mentioned cell line, and a reverse transcription was carried out (TaqMan Reverse Transcription Reagents kit, Applied Biosystems Inc.) using this total RNA (400 ng) as a template and a random primer to prepare cDNA. For the formulation of the reaction solution for the quantitative PCR reaction, 1 μL of the above-mentioned cDNA as a template, 10 μL of TaqMan™ Universal PCR Master Mix (Applied Biosystems Inc.), and 2 μL of TaqMan™ Gene Expression Assay (Applied Biosystems Inc.) containing each primer and TaqMan probe as a set were included to prepare a 20 μL reaction solution in total. In the PCR reaction, after 2 minutes at 50° C. and 10 minutes at 95° C., a cycle of 15 seconds at 95° C. and 1 minute at 60° C. was repeated 40 times. As an internal standard, correction with the expression value of GAPDH gene was done and then each expression value of COLO205 cell line was set as 1 to determine the value of their gene expression level. With regard to the groups of TRAILR1 agonist antibody sensitive strain and TRAILR1 agonist antibody insensitive strain, Student's t-test was subjected using SAS Preclinical Package, Version 5.0 (SAS Institute Japan). As a result, significant values of p<0.01 with STK17B (SEQ ID NO: 1, 2) and LOC93349 (SEQ ID NO: 3, 4) and p<0.05 with CASP8 (SEQ ID NO: 5, 6), SP110 (SEQ ID NO: 7, 8), and NOD27 (SEQ ID NO: 9, 10) were obtained. In addition, AIM1 (SEQ ID NO: 11, 12) and RHOBTB3 (SEQ ID NO: 13, 14) were selected as a suitable type upon the sensitive/insensitive classification carried out using a determination tree procedure. With the use of one or plural number of these genes, it was thought as that the sensitivity of TRAIL-induced apoptosis can be predicted by subjecting the measurement on the expression level, the protein amount, or the activity.

### Example 4(1)

### Setting of Criteria for Sensitivity Prediction

From the genes described in Example 3, following criteria for sensitivity prediction were examined on the basis of the expression values in learning cell lines (Table 1). As a result, four genes of STK17B, LOC93349, CASP8, and AIM1 were selected and the criteria for sensitivity prediction were set as follows. As an internal standard, when correction with the expression value of GAPDH gene was done and then each expression value of COLO205 cell line was set as 1, (1) values of expression relative level of AIM1 in colon cancer and breast cancer of 2.2 or greater was ranked as insensitive without any conditions; (2) samples excluded from (1) were applied to a point system, and the values of expression relative level of SKT17B, LOC93349, and CASP8 of 0.7 or greater were ranked as ‘+1’ and 0.5 or less were ranked as ‘−1’, respectively. The expression value between 0.5 and 0.7 was ranked as ‘0’. Finally, when the sum of all points was positive, ranked as sensitive and when it was negative, ranked as insensitive. The value of ‘0’ was defined as ‘impossible to evaluate’. As a result of applying the criteria for sensitivity prediction, 26 strains out of 28 strains can be predicted in the learning samples described in Example 1, and the sensitivity of 25 strains out of these 26 strains can be accurately predicted (Table 2).

TABLE 2
Preliminary Results for Learning Cell lines

| | Cell | | | | | | | | Rate of |
|---|---|---|---|---|---|---|---|---|---|
| | Lines | Sensitivity | AIM1 | STK17B | LOC93349 | CASP8 | point | Prediction | correction |
| Colon | COLO205 | S | 1.00 | 1.00 | 1.00 | 1.00 | +3 | S | 10/10 |
| Cancer | HCT15 | S | 0.52 | 3.46 | 0.53 | 1.38 | +2 | S | |
| | HCT116 | S | 0.18 | 0.52 | 0.96 | 0.71 | +2 | S | |
| | DLD1 | S | 1.93 | 2.34 | 0.48 | 1.28 | +1 | S | |
| | SW480 | S | 0.03 | 0.66 | 0.23 | 0.78 | 0 | — | |
| | COLO201 | S | 1.86 | 1.44 | 1.14 | 1.26 | +3 | S | |
| | SW48 | S | 0.66 | 1.54 | 0.72 | 0.39 | +1 | S | |
| | COLO320DM | R | 0.26 | 0.32 | 0.00 | 0.15 | −3 | R | |
| | SW620 | R | 0.03 | 0.23 | 0.22 | 0.23 | −3 | R | |
| | WiDr | R | 2.97 | 1.03 | 1.33 | 1.60 | | R | |
| | LS180 | R | 2.44 | 0.61 | 0.63 | 0.66 | | R | |
| Lung | NCI-H2122 | S | 0.54 | 1.71 | 0.95 | 1.45 | +3 | S | 6/7 |
| Cancer | NCI-H460 | S | 0.07 | 0.89 | 1.03 | 1.08 | +3 | S | |
| | NCI-H358 | S | 2.23 | 1.44 | 1.10 | 1.23 | +3 | S | |
| | PC-14 | S | 0.97 | 0.72 | 0.29 | 0.15 | −1 | R | |
| | NCI-H23 | R | 0.02 | 0.23 | 0.00 | 0.10 | −3 | R | |
| | A549 | R | 0.03 | 0.31 | 0.14 | 0.45 | −3 | R | |
| | NCI-H1703 | R | 0.00 | 0.46 | 0.35 | 0.21 | −3 | R | |
| Breast | MDA-MB- | S | 0.42 | 1.15 | 0.61 | 0.58 | +1 | S | 8/8 |
| Cancer | 231 | | | | | | | | |
| | MDA-MB- | S | 1.56 | 1.14 | 1.66 | 1.11 | +3 | S | |
| | 435S | | | | | | | | |
| | MDA-MB- | S | 0.14 | 1.13 | 0.25 | 0.81 | +1 | S | |
| | 436 | | | | | | | | |
| | MDA-MB- | S | 1.93 | 5.78 | 1.89 | 3.15 | +3 | S | |
| | 175VII | | | | | | | | |
| | MCF7 | R | 1.11 | 0.84 | 0.44 | 0.51 | 0 | — | |
| | BT474 | R | 3.78 | 1.13 | 0.02 | 1.40 | | R | |
| | SKBr3 | R | 1.68 | 0.36 | 0.03 | 0.42 | −3 | R | |
| | MDA-MB- | R | 3.37 | 0.39 | 0.76 | 0.73 | | R | |
| | 468 | | | | | | | | |
| | Zr75-1 | R | 2.27 | 0.69 | 0.02 | 0.23 | | R | |
| Stomach | SNU-668 | S | 0.36 | 1.62 | 0.72 | 0.48 | +1 | S | 1/1 |
| Cancer | | | | | | | | | |

### Example 4(2)

### Setting of Criteria for Sensitivity Prediction

From the genes described in Example 3, following criteria for sensitivity prediction were examined on the basis of the expression values in learning cell lines (Table 1). As a result, four genes of STK17B, LOC93349, CASP8, and AIM1 were selected and the criteria for sensitivity prediction were set as follows. As an internal standard, when correction with the expression value of GAPDH gene was done and then each expression value of COLO205 cell line was set as 1, (1) values of expression relative level of AIM1 in colon cancer and breast cancer of 2.2 or greater were ranked as insensitive without any conditions; (2) samples excluded from (1) were applied to a point system, and the values of relative expression level of SKT17B of 1.036 or greater was ranked as ‘+1’ and 0.514 or less was ranked as ‘-1’, and the values of expression of between 1.036 and 0.514 was ranked as ‘0’.

The values of relative expression level of LOC93349 of 0.666 or greater was ranked as ‘+1’ and 0.211 or less was ranked as ‘−1’, and the values of expression of between 0.666 and 0.211 was ranked as ‘0’.

The values of relative expression level of CASP8 of 0.833 or greater was ranked as ‘+1’ and 0.519 or less was ranked as ‘−1’, and the values of expression of between 0.833 and 0.519 was ranked as ‘0’.

Finally, when the sum of all points was positive, ranked as sensitive and when it was negative, ranked as insensitive. The value of ‘0’ was defined as ‘impossible to evaluate’. As a result of applying the criteria for sensitivity prediction, 27 strains out of 28 strains could be predicted in the learning samples described in Example 1, and the sensitivity of 26 strains out of these 27 strains could be accurately predicted (Table 2′).

TABLE 2′
Preliminary Results for Learning Cell lines

| | Cell | | | | | | | | Rate of |
|---|---|---|---|---|---|---|---|---|---|
| | Lines | Sensitivity | AIM1 | STK17B | LOC93349 | CASP8 | point | Prediction | correction |
| Colon | COLO205 | S | 1.00 | 1.00 | 1.00 | 1.00 | +2 | S | 10/10 |
| Cancer | HCT15 | S | 0.52 | 3.46 | 0.53 | 1.38 | +2 | S | |
| | HCT116 | S | 0.18 | 0.52 | 0.96 | 0.71 | +1 | S | |
| | DLD1 | S | 1.93 | 2.34 | 0.48 | 1.28 | +2 | S | |
| | SW480 | S | 0.03 | 0.66 | 0.23 | 0.78 | 0 | — | |
| | COLO201 | S | 1.86 | 1.44 | 1.14 | 1.26 | +3 | S | |
| | SW48 | S | 0.66 | 1.54 | 0.72 | 0.39 | +1 | S | |
| | COLO320DM | R | 0.26 | 0.32 | 0.00 | 0.15 | −3 | R | |
| | SW620 | R | 0.03 | 0.23 | 0.22 | 0.23 | −2 | R | |
| | WiDr | R | 2.97 | 1.03 | 1.33 | 1.60 | | R | |
| | LS180 | R | 2.44 | 0.61 | 0.63 | 0.66 | | R | |
| Lung | NCI-H2122 | S | 0.54 | 1.71 | 0.95 | 1.45 | +3 | S | 6/7 |
| Cancer | NCI-H460 | S | 0.07 | 0.89 | 1.03 | 1.08 | +2 | S | |
| | NCI-H358 | S | 2.23 | 1.44 | 1.10 | 1.23 | +3 | S | |
| | PC-14 | S | 0.97 | 0.72 | 0.29 | 0.15 | −1 | R | |
| | NCI-H23 | R | 0.02 | 0.23 | 0.00 | 0.10 | −3 | R | |
| | A549 | R | 0.03 | 0.31 | 0.14 | 0.45 | −3 | R | |
| | NCI-H1703 | R | 0.00 | 0.46 | 0.35 | 0.21 | −2 | R | |
| Breast | MDA-MB- | S | 0.42 | 1.15 | 0.61 | 0.58 | +1 | S | 9/9 |
| Cancer | 231 | | | | | | | | |
| | MDA-MB- | S | 1.56 | 1.14 | 1.66 | 1.11 | +3 | S | |
| | 435S | | | | | | | | |
| | MDA-MB- | S | 0.14 | 1.13 | 0.25 | 0.81 | +1 | S | |
| | 436 | | | | | | | | |
| | MDA-MB- | S | 1.93 | 5.78 | 1.89 | 3.15 | +3 | S | |
| | 175VII | | | | | | | | |
| | MCF7 | R | 1.11 | 0.84 | 0.44 | 0.51 | −1 | R | |
| | BT474 | R | 3.78 | 1.13 | 0.02 | 1.40 | | R | |
| | SKBr3 | R | 1.68 | 0.36 | 0.03 | 0.42 | −3 | R | |
| | MDA-MB- | R | 3.37 | 0.39 | 0.76 | 0.73 | | R | |
| | 468 | | | | | | | | |
| | Zr75-1 | R | 2.27 | 0.69 | 0.02 | 0.23 | | R | |
| Stomach | SNU-668 | S | 0.36 | 1.62 | 0.72 | 0.48 | +1 | S | 1/1 |
| Cancer | | | | | | | | | |

### Example 4(3)

### Setting of Criteria for Sensitivity Prediction

From the genes described in Example 3, following criteria for sensitivity prediction were examined on the basis of the expression values in learning cell lines (Table 1). As a result, four genes of STK17B, LOC93349, CASP8, and AIM1 were selected and the criteria for sensitivity prediction were set as follows. As an internal standard, when correction with the expression value of GAPDH gene was done and then each expression value of COLO205 cell line was set as 1, (1) values of expression relative level of AIM1 in colon cancer and breast cancer of 2.2 or greater was ranked as TRAIL signal activator insensitive, without any conditions.

The reference value for STK17B, LOC93349 and CASP8 was the median value of each relative expression level (two places of decimals), and when two or more of respective relative expression levels were not less than the reference value, the cell line was ranked as TRAIL signal activator sensitive. When two or more of respective relative expression levels were less than the reference value, the cell line was ranked as TRAIL signal activator insensitive. The reference value (median value) for STK17B was 0.94, the reference value (median value) for LOC93349 was 0.57, and the reference value (median value) for CASP8 was 0.72. As a result of applying the criteria for sensitivity prediction, the sensitivity of 25 cell lines out of 28 cell lines can be accurately predicted (Table 2″).

TABLE 2″
Preliminary Results for Learning Cell lines

| | | | | | | | | Rate of |
|---|---|---|---|---|---|---|---|---|
| | Cell Lines | Sensitivity | AIM1 | STK17B | LOC93349 | CASP8 | Prediction | Correction |
| Colon | COLO205 | S | 1.00 | 1.00 | 1.00 | 1.00 | S | 9/11 |
| Cancer | HCT15 | S | 0.52 | 3.46 | 0.53 | 1.38 | S | |
| | HCT116 | S | 0.18 | 0.52 | 0.96 | 0.71 | R | |
| | DLD1 | S | 1.93 | 2.34 | 0.48 | 1.28 | S | |
| | SW480 | S | 0.03 | 0.66 | 0.23 | 0.78 | R | |
| | COLO201 | S | 1.86 | 1.44 | 1.14 | 1.26 | S | |
| | SW48 | S | 0.66 | 1.54 | 0.72 | 0.39 | S | |
| | COLO320DM | R | 0.26 | 0.32 | 0.00 | 0.15 | R | |
| | SW620 | R | 0.03 | 0.23 | 0.22 | 0.23 | R | |
| | WiDr | R | 2.97 | 1.03 | 1.33 | 1.60 | R | |
| | LS180 | R | 2.44 | 0.61 | 0.63 | 0.66 | R | |
| Lung | NCI-H2122 | S | 0.54 | 1.71 | 0.95 | 1.45 | S | 6/7 |
| Cancer | NCI-H460 | S | 0.07 | 0.89 | 1.03 | 1.08 | S | |
| | NCI-H358 | S | 2.23 | 1.44 | 1.10 | 1.23 | S | |
| | PC-14 | S | 0.97 | 0.72 | 0.29 | 0.15 | R | |
| | NCI-H23 | R | 0.02 | 0.23 | 0.00 | 0.10 | R | |
| | A549 | R | 0.03 | 0.31 | 0.14 | 0.45 | R | |
| | NCI-H1703 | R | 0.00 | 0.46 | 0.35 | 0.21 | R | |
| Breast | MDA-MB-231 | S | 0.42 | 1.15 | 0.61 | 0.58 | S | 9/9 |
| Cancer | MDA-MB- | S | 1.56 | 1.14 | 1.66 | 1.11 | S | |
| | 435S | | | | | | | |
| | MDA-MB-436 | S | 0.14 | 1.13 | 0.25 | 0.81 | S | |
| | MDA-MB- | S | 1.93 | 5.78 | 1.89 | 3.15 | S | |
| | 175VII | | | | | | | |
| | MCF7 | R | 1.11 | 0.84 | 0.44 | 0.51 | R | |
| | BT474 | R | 3.78 | 1.13 | 0.02 | 1.40 | R | |
| | SKBr3 | R | 1.68 | 0.36 | 0.03 | 0.42 | R | |
| | MDA-MB-468 | R | 3.37 | 0.39 | 0.76 | 0.73 | R | |
| | Zr75-1 | R | 2.27 | 0.69 | 0.02 | 0.23 | R | |
| Stomach | SNU-668 | S | 0.36 | 1.62 | 0.72 | 0.48 | S | 1/1 |
| Cancer | | | | | | | | |

### Example 5

### Measurement of Sensitivity of TRAILR1 Agonist Antibody in Cell Line for Verification

For cell line for verification, 14 strains of human cancer cell lines, that is, large intestine cell line; SW1116, SW1417, SW403, SW837, and SW948, lung cancer cell line; NCI-H838, NCI-H226, NCI-H520, NCI-H522, and NCI-H2347, and breast cancer cell line; T47D, BT549, MDA-MB-157, and MDA-MB-361, were used (place to obtain is shown in Table 3). As the medium, RPMI 1640 medium (SIGMA) was used for NCI-H838, NCI-H226, NCI-H520, NCI-H522, NCI-H2347, T47D, and BT549, and Leibovitz's L-15 medium (Invitrogen) was used for SW1116, SW1417, SW403, SW837, SW948, and MDA-MB-157 and MDA-MB-361. Thereto, 10% fetal bovine serum (JRH) and 50 μg/ml penicillin/streptomycin (Invitrogen) were added. Cell lines were cultured at 37° C., in the atmosphere of 5% CO₂ when RPMI 1640 medium was used, but in natural airflow when Leibovitz's L-15 medium was used.

Measurement of the TRAIL agonist antibody sensitivity of the cells for verification was carried out according to the method described in Example 1. Specifically, the respective cells were harvested using trypsin/ETDA (Invitrogen), suspended in 50 μl of the respective culture solutions at a concentration of 5.0×10³ cells/50 μl, and inoculated to each well of a 96-well plate in an amount of 50 μl each. At this time, the cell lines cultured in Leibovitz's L-15 medium were transferred to Dulbecco's modified Eagle's medium, and all were cultured at 37° C. under a 5% carbon dioxide stream. After 24 hours, 50 μl of TRAILR1 agonist antibody diluted with the same culture solutions as those used for the respective cell cultures, was added in three portions from 39.6 nM to a common ratio of 12-fold. After 48 hours, 10 μl of cell counting kit-8 (Dojin) was added for the WST-8 assay as in Example 1, and after 3 hours, the absorbance at 450 nm was measured as an index to the number of live cells. The absorbance obtained under no addition of the TRAILR1 agonist antibody was taken as 100%, and the ratio of live cells was examined. Also, CellTiter-Glo Luminescent Cell viability Assay (Promega) was performed at the same time, to measure the amount of ATP in the cells. The absorbance obtained under no addition of the TRAILR1 agonist antibody was taken as 100%, and the ratio of live cells was examined. From the above-described WST-8 or ATP assay, cell lines having cell growth inhibitory rates of 30% or greater when the concentration of the TRAILR1 agonist antibody was 39.6 nM, were determined to be TRAILR1 agonist antibody sensitive cell lines (Table 3).

TABLE 3
Purchased Place and TRAILR1 agonist antibody
sensitivity of Cell lines for Verification

| | | | | GI rate (%) |
|---|---|---|---|---|
| | Cell Lines | Purchased Place | Sensitivity | (39.6 nM) |
| Colon | SW1116 | DAINIPPON SUMITOMO PHARMA | S | 56.6 |
| Cancer | SW1417 | DAINIPPON SUMITOMO PHARMA | R | 6.2 |
| | SW403 | DAINIPPON SUMITOMO PHARMA | R | 1.8 |
| | SW837 | DAINIPPON SUMITOMO PHARMA | R | 3.9 |
| | SW948 | DAINIPPON SUMITOMO PHARMA | S | 99.2 |
| Lung | NCI-H838 | American Type Culture | S | 56.1 |
| Cancer | | Collection | | |
| | NCI-H226 | American Type Culture | R | 22.1 |
| | | Collection | | |
| | NCI-H520 | American Type Culture | R | 25.1 |
| | | Collection | | |
| | NCI-H522 | American Type Culture | R | 14.0 |
| | | Collection | | |
| | NCI-H2347 | American Type Culture | S | 34.5 |
| | | Collection | | |
| Breast | T47D | DAINIPPON SUMITOMO PHARMA | R | 3.4 |
| Cancer | BT-549 | DAINIPPON SUMITOMO PHARMA | R | 0.0 |
| | MDA-MB-157 | DAINIPPON SUMITOMO PHARMA | S | 38.3 |
| | MDA-MB-361 | DAINIPPON SUMITOMO PHARMA | R | 0.0 |

### Example 6(1)

### Verification of Criteria for Sensitivity

The criteria described in Example 4(1) were used to verify whether the sensitivity of the cells for verification described in Example 5 can be predicted. For the cells for verification described in Example 5, that is, 14 cell lines, cDNAs were prepared in the same manner as in Example 3, and relative expression levels of four genes, STK17B, LOC93349, CASP8, and AIM1, were determined by the quantitative PCR reaction. As the result of application of the criteria for predicting sensitivity, it was found that the sensitivity of TRAILR1 agonist antibody of 12 cell lines out of 14 cell lines can be predicted, and the predicted results are matching to the experimental results in 9 cell lines out of 12 cell lines, and thus the samples for verification can be predicted with 75% precision (Table 4).

As described above, it was indicated that prediction of sensitivity to TRAIL induction apoptosis is possible by comparing expression level of genes selected from genes described in Example 2.

TABLE 4
Prediction of TRAILR1 agonist antibody sensitivity in
samples for Verification

| | Cell | | | | | | | | Rate of |
|---|---|---|---|---|---|---|---|---|---|
| | Lines | Sensitivity | AIM1 | STK17B | LOC93349 | CASP8 | Point | Prediction | Correction |
| Colon | SW1116 | S | 1.12 | 0.38 | 0.31 | 0.56 | −2 | R | 3/4 |
| Cancer | SW1417 | R | 0.66 | 1.82 | 0.17 | 0.58 | 0 | — | |
| | SW403 | R | 2.24 | 1.81 | 0.66 | 1.13 | | R | |
| | SW837 | R | 2.23 | 3.41 | 0.83 | 1.29 | | R | |
| | SW948 | S | 1.89 | 3.12 | 0.18 | 2.79 | +1 | S | |
| Lung | NCI-H838 | S | 0.69 | 0.49 | 0.39 | 0.59 | −1 | R | 3/5 |
| Cancer | NCI-H226 | R | 0.01 | 0.21 | 1.01 | 0.10 | −1 | R | |
| | NCI-H520 | R | 1.52 | 0.30 | 0.52 | 0.11 | −2 | R | |
| | NCI-H522 | R | 0.00 | 0.00 | 0.01 | 0.10 | −3 | R | |
| | NCI-H2347 | S | 1.14 | 0.94 | 0.14 | 0.19 | −1 | R | |
| Breast | T47D | R | 5.78 | 0.92 | 1.18 | 0.38 | | R | 3/3 |
| Cancer | BT-549 | R | 0.52 | 1.24 | 0.08 | 0.45 | −1 | R | |
| | MDA-MB- | S | 0.00 | 6.08 | 1.11 | 1.95 | +3 | S | |
| | 157 | | | | | | | | |
| | MDA-MB- | R | 1.73 | 0.70 | 0.09 | 0.59 | 0 | — | |
| | 361 | | | | | | | | |

### Example 6(2)

### Verification of Criteria for Sensitivity

The criteria described in Example 4(2) were used to verify whether the sensitivity of the cells for verification described in Example 5 can be predicted. For the cells for verification described in Example 5, that is, 14 cell lines, cDNAs were prepared in the same manner as in Example 3, and relative expression levels of four genes, STK17B, LOC93349, CASP8, and AIM1, were determined by the quantitative PCR reaction. As the result of application of the criteria for predicting sensitivity, it was found that the sensitivity of TRAILR1 agonist antibody of 13 cell lines out of 14 cell lines can be predicted, and the predicted results are matching to the experimental results in 10 cell lines out of 13 cell lines, and thus the samples for verification can be predicted with 76.9% precision (Table 4′).

TABLE 4′
Prediction of TRAILR1 agonist antibody sensitivity
in samples for Verification

| | | | | | | | | | Rate of |
|---|---|---|---|---|---|---|---|---|---|
| | Cell Lines | Sensitivity | AIM1 | STK17B | LOC93349 | CASP8 | Point | Prediction | Correction |
| Colon | SW1116 | S | 1.12 | 0.38 | 0.31 | 0.56 | −1 | R | 3/4 |
| Cancer | SW1417 | R | 0.66 | 1.82 | 0.17 | 0.58 | 0 | — | |
| | SW403 | R | 2.24 | 1.81 | 0.66 | 1.13 | | R | |
| | SW837 | R | 2.23 | 3.41 | 0.83 | 1.29 | | R | |
| | SW948 | S | 1.89 | 3.12 | 0.18 | 2.79 | +1 | S | |
| Lung | NCI-H838 | S | 0.69 | 0.49 | 0.39 | 0.59 | −1 | R | 3/5 |
| Cancer | NCI-H226 | R | 0.01 | 0.21 | 1.01 | 0.10 | −1 | R | |
| | NCI-H520 | R | 1.52 | 0.30 | 0.52 | 0.11 | −2 | R | |
| | NCI-H522 | R | 0.00 | 0.00 | 0.01 | 0.10 | −3 | R | |
| | NCI-H2347 | S | 1.14 | 0.94 | 0.14 | 0.19 | −2 | R | |
| Breast | T47D | R | 5.78 | 0.92 | 1.18 | 0.38 | | R | 4/4 |
| Cancer | BT-549 | R | 0.52 | 1.24 | 0.08 | 0.45 | −1 | R | |
| | MDA-MB-157 | S | 0.00 | 6.08 | 1.11 | 1.95 | +3 | S | |
| | MDA-MB-361 | R | 1.73 | 0.70 | 0.09 | 0.59 | −1 | R | |

### Example 6(3)

### Verification of Criteria for Sensitivity

The criteria described in Example 4(3) were used to verify whether the sensitivity of the cells for verification described in Example 5 can be predicted. For the cells for verification described in Example 5, that is, 14 cell lines, cDNAs were prepared in the same manner as in Example 3, and relative expression levels of four genes, STK17B, LOC93349, CASP8, and AIM1, were determined by the quantitative PCR reaction. As the result of application of the criteria for predicting sensitivity, the predicted results are matching with 11 cell lines out of 14 cell lines in the experimental results (Table 4″).

TABLE 4″
Prediction of TRAILR1 agonist antibody sensitivity
in samples for Verification

| | | | | | | | | Rate of |
|---|---|---|---|---|---|---|---|---|
| | Cell Lines | Sensitivity | AIM1 | STK17B | LOC93349 | CASP8 | Prediction | Correction |
| Colon | SW1116 | S | 1.12 | 0.38 | 0.31 | 0.56 | R | 4/5 |
| Cancer | SW1417 | R | 0.66 | 1.82 | 0.17 | 0.58 | R | |
| | SW403 | R | 2.24 | 1.81 | 0.66 | 1.13 | R | |
| | SW837 | R | 2.23 | 3.41 | 0.83 | 1.29 | R | |
| | SW948 | S | 1.89 | 3.12 | 0.18 | 2.79 | S | |
| Lung | NCI-H838 | S | 0.69 | 0.49 | 0.39 | 0.59 | R | 3/5 |
| Cancer | NCI-H226 | R | 0.01 | 0.21 | 1.01 | 0.10 | R | |
| | NCI-H520 | R | 1.52 | 0.30 | 0.52 | 0.11 | R | |
| | NCI-H522 | R | 0.00 | 0.00 | 0.01 | 0.10 | R | |
| | NCI-H2347 | S | 1.14 | 0.94 | 0.14 | 0.19 | R | |
| Breast | T47D | R | 5.78 | 0.92 | 1.18 | 0.38 | R | 4/4 |
| Cancer | BT-549 | R | 0.52 | 1.24 | 0.08 | 0.45 | R | |
| | MDA-MB-157 | S | 0.00 | 6.08 | 1.11 | 1.95 | S | |
| | MDA-MB-361 | R | 1.73 | 0.70 | 0.09 | 0.59 | R | |

As described above, it was indicated that prediction of sensitivity to TRAIL induction apoptosis is possible by comparing expression level of genes selected from genes described in Example 2.

### Example 7

### Involvement of STK17B in TRAIL Signal

In order to determine whether STK17B gene selected as a marker sensitive to TRAILR1 agonist antibody is in relation with the sensitivity to TRAILR1 agonist antibody, siRNA for STK17B gene was transformed into a strain sensitive to TRAILR1 agonist antibody to confirm whether the sensitivity is decreased. As cell lines, lung cancer cell line; H460 and large intestine cell line; HCT116 and SW480, which are cell lines sensitive to TRAILR1 agonist antibody, were used, the cell lines were cultured by the method described in Example 1. Each cell was collected using trypsin/EdTA (Invitrogen), collected cell was suspended in each culture so as to be 2.5×10³/100 μl, and seeded into a 96-well plate to give 100 μl per each well. After 24-hour culturing, 289.5 μl of OPTI-MEM (Invitrogen), 7.5 μl of Lipofectamine RNAiMAX (Invitrogen), and 3 μl of siRNA (ON TARGETplus SMARTpool siRNA; Dharmacon, 50 μM) for STK17B gene, or 3 μl of Non Silencing siRNA (ON TARGETplus SMARTpool siRNA; and Dharmacon, 50 μM) for STK17B gene were mixed and left at rest at room temperature for 20 minutes. The suspension was added to each seeded cell line by 10 μl to transfer siRNA. For the measurement of apoptosis, Cell Death Detection ELISA (Roche) was used. The expression level of STK17B gene was measured by the method described in Example 3. For method of Western analysis, cell lines were washed with PBS, and then protein was collected by the use of RIPA Buffer (50 mM Tris-HCl (pH 7.6), 150 mM NaCl, 1% TritonX-100, Protease Inhibitor Cocktail Tablets). 5× Sample Buffer (125 mM Tris-HCl (pH 6.8), 50% glycerol, 5% SDS, 0.02% Bromophenol Blue) was added to the collected samples. Thereafter, the samples were subjected to denaturation with heat at 100° C. for 3 minutes, and then subjected to electrophoresis with 10% acrylamide gel (ATTO Corporation) (20 mA, 80 minutes). The electrophoresed protein was transferred from a gel to a PVDF membrane (ATTO Corporation) (200 mA, 60 minutes), and then blocking was subjected by the use of block ace (Dainippon Sumitomo Pharma Co., Ltd) at a room temperature for 60 minutes. A primary antibody was reacted with 3 μg/ml of anti STK17B antibody (Santa cruz biotechnology, inc) at room temperature for 60 minutes, and washed three times with TBST for 3 minutes per each time. A secondary antibody was reacted with anti goat IgG-HRP (1,000-fold dilution, Santa cruz biotechnology, inc), at a room temperature for 60 minutes, and washed three times with TBST for 3 minutes per each time. Thereafter, band to be targeted was detected by chemiluminescence (ECL Plus Western Blotting Detection System, GE Healthcare Bioscience).

To the cells 72 hours after transfecting siRNA, TRAILR1 agonist antibody (final concentration of 6 nM and 30 nM) was added, and assayed 3 hours after the addition. As a result, it was confirmed that RNA and protein of STK17B decreases by 90% or more in any cell line to which siRNA for STK17B gene had been transfected, of lung cancer cell line; H460, and large intestine cell line; HCT116 and SW480, apoptosis due to TRAILR1 agonist antibody decreases by 55.9% in H460, 40.2% in HCT116, 28.4% in SW480 in the case of the final concentration of 6 nM, while the apoptosis decreases by 44.1%, 29.1%, 25.9%, and sensitivity of the TRAILR1 agonist antibody decreases. From the results, it was found that STK17B protein plays an important role in the sensitivity to TRAILR1 agonist antibody and TRAIL signal, and thus sensitivity of TRAIL induction apoptosis is increased by promoting the expression of STK17B.

### Example 8

### Investigation of TRAIL Sensitivity in Human Cancer Cell Lines

Using the same method as in Example 1, the TRAIL sensitivity in the cell lines indicated in Table 1 (excluding MDA-MB-175 VII) was investigated. Cell lines having an IC₅₀ of 1 nM or less were determined to be TRAIL sensitive. Table 5 shows a summary of IC₅₀, resistance, sensitivity, and the difference between the TRAIL sensitivity and TRAILR1 agonist antibody sensitivity. The sensitivity to TRAILR1 agonist antibody and the sensitivity to TRAIL were identical in 24 cell lines among 27 cell lines. As such, it was found that the sensitivity to TRAILR1 agonist antibody and the sensitivity to TRAIL are similar in a plurality of carcinomas and cell lines.

TABLE 5
TRAIL sensitivity of various Cancer Cell lines

| | | | | Difference |
|---|---|---|---|---|
| | | | | from TRAILR1 |
| | | | | Agonist |
| Kind of | | | | Antibody |
| Cancer | Cell Lines | IC50 (nM) | Sensitivity*1 | Sensitivity*2 |
| Colon | COLO205 | 0.005 | S | ◯ |
| Cancer | HCT15 | 0.042 | S | ◯ |
| | HCT116 | 0.027 | S | ◯ |
| | DLD1 | 0.058 | S | ◯ |
| | SW480 | 0.075 | S | ◯ |
| | COLO201 | 0.12 | S | ◯ |
| | SW48 | 0.031 | S | ◯ |
| | COLO320DM | >26 | R | ◯ |
| | SW620 | 0.13 | S | X |
| | WiDr | 0.067 | S | X |
| | LS180 | 5.6 | R | ◯ |
| Lung | NCI-H2122 | 0.03 | S | ◯ |
| Cancer | NCI-H460 | 0.004 | S | ◯ |
| | NCI-H358 | 0.046 | S | ◯ |
| | PC-14 | 0.085 | S | ◯ |
| | NCI-H23 | >26 | R | ◯ |
| | A549 | 4.6 | R | ◯ |
| | NCI-H1703 | 0.11 | S | X |
| Breast | MDA-MB-231 | 0.83 | S | ◯ |
| Cancer | MDA-MB-435S | 0.13 | S | ◯ |
| | MDA-MB-436 | 0.27 | S | ◯ |
| | MCF7 | >26 | R | ◯ |
| | BT474 | >26 | R | ◯ |
| | SKBr3 | >26 | R | ◯ |
| | MDA-MB-468 | 7.4 | R | ◯ |
| | Zr75-1 | 7.6 | R | ◯ |
| Stomach | SNU-668 | 0.024 | S | ◯ |
| Cancer | | | | |

Sensitivity*: those with IC50 value of 1 nM or less are ranked as sensitive strain (S) and those with equal or greater than that value are ranked as resistant strain (R)
Difference from TRAILR1 agonist antibody sensitivity*2: those identical to the TRAILR1 agonist sensitivity are ranked as ◯, and those not identical are ranked as X.

### Example 9(1)

### Verification of Criteria for TRAIL Sensitivity

The criteria for predicting sensitivity described in Example 4(1) were used to verify whether TRAIL sensitivity of the cells described in Example 7 can be predicted. As a result, it was found that 25 cell lines out of 27 cell lines can be predicted, and TRAIL sensitivity in 21 cell lines out of 25 cell lines can be accurately predicted (Table 6). It was shown that the prediction of TRAIL sensitivity is possible by the use of the criteria for TRAIL sensitivity, as described above.

TABLE 6
Prediction Results for Learning Cell lines

| | | TRAIL | | Rate of |
|---|---|---|---|---|
| | Cell Lines | Sensitivity | Prediction | Correction |
| Colon | COLO205 | S | S | 8/10 |
| Cancer | HCT15 | S | S | |
| | HCT116 | S | S | |
| | DLD1 | S | S | |
| | SW480 | S | — | |
| | COLO201 | S | S | |
| | SW48 | S | S | |
| | COLO320DM | R | R | |
| | SW620 | S | R | |
| | WiDr | S | R | |
| | LS180 | R | R | |
| Lung | NCI-H2122 | S | S | 5/7 |
| Cancer | NCI-H460 | S | S | |
| | NCI-H358 | S | S | |
| | PC-14 | S | R | |
| | NCI-H23 | R | R | |
| | A549 | R | R | |
| | NCI-H1703 | S | R | |
| Breast | MDA-MB-231 | S | S | 7/7 |
| Cancer | MDA-MB- | S | S | |
| | 435S | S | S | |
| | MDA-MB-436 | R | — | |
| | MCF7 | R | R | |
| | BY474 | R | R | |
| | SKBr3 | R | R | |
| | MDA-MB-468 | R | R | |
| | Zr75-1 | | | |
| Stomach | SNU-668 | S | S | 1/1 |
| Cancer | | | | |

### Example 9(2)

### Verification of Criteria for TRAIL Sensitivity

The criteria for predicting sensitivity described in Example 4(2) were used to verify whether TRAIL sensitivity of the cells described in Example 7 can be predicted. As a result, it was found that 26 cell lines out of 27 cell lines can be predicted, and TRAIL sensitivity in 22 cell lines out of 26 cell lines can be accurately predicted (Table 6′). It was shown that the prediction of TRAIL sensitivity is possible by the use of the criteria for TRAIL sensitivity, as described above.

TABLE 6′
Prediction Results for Learning Cell lines

| | | TRAIL | | Rate of |
|---|---|---|---|---|
| | Cell Lines | Sensitivity | Prediction | Correction |
| Colon | COLO205 | S | S | 8/10 |
| Cancer | HCT15 | S | S | |
| | HCT116 | S | S | |
| | DLD1 | S | S | |
| | SW480 | S | — | |
| | COLO201 | S | S | |
| | SW48 | S | S | |
| | COLO320DM | R | R | |
| | SW620 | S | R | |
| | WiDr | S | R | |
| | LS180 | R | R | |
| Lung | NCI-H2122 | S | S | 5/7 |
| Cancer | NCI-H460 | S | S | |
| | NCI-H358 | S | S | |
| | PC-14 | S | R | |
| | NCI-H23 | R | R | |
| | A549 | R | R | |
| | NCI-H1703 | S | R | |
| Breast | MDA-MB-231 | S | S | 8/8 |
| Cancer | MDA-MB- | S | S | |
| | 435S | S | S | |
| | MDA-MB-436 | R | R | |
| | MCF7 | R | R | |
| | BY474 | R | R | |
| | SKBr3 | R | R | |
| | MDA-MB-468 | R | R | |
| | Zr75-1 | | | |
| Stomach | SNU-668 | S | S | 1/1 |
| Cancer | | | | |

### Example 9(3)

### Verification of Criteria for TRAIL Sensitivity

The criteria for predicting sensitivity described in Example 4(3) were used to verify whether TRAIL sensitivity of the cells described in Example 7 can be predicted. As a result, TRAIL sensitivity in 21 cell lines out of 27 cell lines can be accurately predicted (Table 6″). It was shown that the prediction of TRAIL sensitivity is possible by the use of the criteria for TRAIL sensitivity, as described above.

TABLE 6″
Prediction Results for Learning Cell lines

| | | TRAIL | | Rate of |
|---|---|---|---|---|
| | Cell Lines | Sensitivity | Prediction | Correction |
| Colon Cancer | COLO205 | S | S | 7/11 |
| | HCT15 | S | S | |
| | HCT116 | S | R | |
| | DLD1 | S | S | |
| | SW480 | S | R | |
| | COLO201 | S | S | |
| | SW48 | S | S | |
| | COLO320DM | R | R | |
| | SW620 | S | R | |
| | WiDr | S | R | |
| | LS180 | R | R | |
| Lung Cancer | NCI-H2122 | S | S | 5/7 |
| | NCI-H460 | S | S | |
| | NCI-H358 | S | S | |
| | PC-14 | S | R | |
| | NCI-H23 | R | R | |
| | A549 | R | R | |
| | NCI-H1703 | S | R | |
| Breast Cancer | MDA-MB-231 | S | S | 8/8 |
| | MDA-MB-435S | S | S | |
| | MDA-MB-436 | S | S | |
| | MCF7 | R | R | |
| | BT474 | R | R | |
| | SKBr3 | R | R | |
| | MDA-MB-468 | R | R | |
| | Zr75-1 | R | R | |
| Stomach Cancer | SNU-668 | S | S | 1/1 |

### INDUSTRIAL APPLICABILITY

According to the invention, a rapid and simple test on TRAIL signal activator sensitivity becomes possible. In addition, since the preventive/remedy for cancer of the invention is selectively administered to the patient sensitive to TRAIL signal activator, cancers can be effectively prevented and/or treated. Further, since the regulator for TRAIL signal activator sensitivity-related factor of the invention can increase the TRAIL signal activator sensitivity in patients, cancers can be effectively prevented and/or treated, for example, by using in combination with TRAIL signal activator.

### Sequence Listing Free Text

### [SEQ ID NO: 23]

Humanized anti-TRAILR1 scFV antibody

### [SEQ ID NO: 24]

Humanized anti-TRAILR1 scFV antibody

This application is based on a patent application No. 2007-268433 filed in Japan, the contents of which are incorporated in full herein by this reference.

1. (canceled)

2. The method according to claim 19, wherein the TRAIL signal activator sensitive patient is selected on the basis of an index including conditions where (i) the expression or activity of AIM1 is not promoted and (ii) the expression or activity of at least 2 selected from the group consisting of STK17B, LOC93349, CASP8, SP110, NOD27, and RHOBTB3 is promoted, in a sample collected from a test subject.

3. The method according to claim 19, wherein the TRAIL signal activator sensitive patient is selected on the basis of an index including conditions where the expression or activity of AIM1 is decreased, and the expression or activity of STK17B and/or LOC93349 and/or CASP8 is promoted, in sample collected from a test subject.

4. The method according to claim 19, wherein the TRAIL signal activator sensitive patient is selected on the basis of an index including condition where the expression or activity of STK17B is promoted, in sample collected from a test subject.

5. The method according to claim 19, wherein the TRAIL signal activator sensitivity marker includes AIM1, STK17B, LOC93349 and CASP8, and in the case where:
(1) the expression level or activity of AIM1, STK17B, LOC93349 and CASP8 in two or more known TRAIL signal activator sensitive cancer cells and two or more known TRAIL signal activator insensitive cancer cells, is measured, and
(2)(i) from the relationship between the expression level or activity of AIM1 in breast cancer or colon cancer cells, among the TRAIL signal activator sensitive cancer cells and TRAIL signal activator insensitive cancer cells as measured in (1), the value of a relative expression level or relative activity which can be used to accurately determine the presence or absence of the sensitivity to a TRAIL signal activator in a cancer cell, is taken as the reference value; and
(ii) from the relationship between the expression level or activity of STK17B, LOC93349 or CASP8 in the TRAIL signal activator sensitive cancer cells and TRAIL signal activator insensitive cancer cells as measured in (1), and the TRAIL signal activator sensitivity, the value of a relative expression level or relative activity which can be used to accurately determine the TRAIL signal activator sensitivity of a cancer cell, is taken as the upper reference value, while the value of a relative expression level or relative activity which can be used to accurately determine the TRAIL signal activator insensitivity of a cancer cell, is taken as the lower reference value,
a test subject corresponding to the following (a) or (b) is screened as a TRAIL signal activator sensitive patient:
(a) in case the test subject is a patient suffering from breast cancer or colon cancer, the expression level or activity of AIM1 in a sample collected from the test subject is less than the reference value, and given that a +1 point is scored if the expression level or activity of STK17B, LOC93349 or CASP8 in the sample collected from the test subject is equal to or greater than the upper reference value; a zero point is scored if the amount of expression or activity is equal to or greater than the lower reference value and also less than the upper reference value; and a −1 point is scored if the expression level or activity is less than the lower reference value, the sum of the points for STK17B, LOC93349 and CASP8 is a positive value; and
(b) in case the test subject is a patient suffering from a cancer other than breast cancer and colon cancer, given that a +1 point is scored if the expression level or activity of STK17B, LOC93349 or CASP8 in the sample collected from the test subject is equal to or greater than the upper reference value; a zero point is scored if the expression level or activity is equal to or greater than the lower reference value and also less than the upper reference value; and a −1 point is scored if the expression level or activity is less than the lower reference value, the sum of the points for STK17B, LOC93349 and CASP8 is a positive value.

6. The method according to claim 5, wherein the TRAIL signal activator sensitivity marker includes AIM1, STK17B, LOC93349 and CASP8, and in the case where:
(1) the expression level or activity of AIM1, STK17B, LOC93349 and CASP8 in 2 or more known TRAIL signal activator sensitive cancer cells and 2 or more known TRAIL signal activator insensitive cancer cells is measured,
(2)(i) using the expression level or activity of AIM1, STK17B, LOC93349 and CASP8 in one cell arbitrarily selected from TRAIL signal activator sensitive cancer cells and TRAIL signal activator insensitive cancer cells (reference cancer cell) as measured in (1) as 1.0, the relative expression level or relative activity of AIM1, STK17B, LOC93349 and CASP8 in other cancer cells is calculated,
(ii) the relationship between the relative expression level (two places of decimals) or relative activity (two places of decimals) of AIM1 in other cells as measured in (1) and the presence or absence of TRAIL signal activator sensitivity in said cells is compared, the highest relative expression level A or relative activity A in the TRAIL signal activator sensitive cancer cell group is selected, relative expression level B or relative activity B in the TRAIL signal activator insensitive cancer cell group, which is greater than the relative expression level A or relative activity A and the nearest thereto, and the value of relative expression level B or relative activity B less the second place of decimal is taken as the reference value,
(iii) the relationship between the relative expression level (two places of decimals) or relative activity (two places of decimals) of STK17B, LOC93349 and CASP8 in other cells as measured in (1) and the presence or absence of TRAIL signal activator sensitivity in said cells is compared, 80% value of the median value of the relative expression level or relative activity in the TRAIL signal activator sensitive cancer cell group is taken as the upper reference value, and 120% value of the median value of the relative expression level or relative activity in the TRAIL signal activator insensitive cancer cell group is taken as the lower reference value,
a test subject corresponding to the following (a) or (b) is screened as a TRAIL signal activator sensitive patient:
(a) in case the test subject is a patient suffering from breast cancer or colon cancer, the expression level or activity of AIM1 in a sample collected from the test subject is less than the reference value, and given that a +1 point is scored if the expression level or activity of STK17B, LOC93349 or CASP8 in the sample collected from the test subject is equal to or greater than the upper reference value; a zero point is scored if the amount of expression or activity is equal to or greater than the lower reference value and also less than the upper reference value; and a −1 point is scored if the expression level or activity is less than the lower reference value, the sum of the points for STK17B, LOC93349 and CASP8 is a positive value; and
(b) in case the test subject is a patient suffering from a cancer other than breast cancer and colon cancer, given that a +1 point is scored if the expression level or activity of STK17B, LOC93349 or CASP8 in the sample collected from the test subject is equal to or greater than the upper reference value; a zero point is scored if the expression level or activity is equal to or greater than the lower reference value and also less than the upper reference value; and a −1 point is scored if the expression level or activity is less than the lower reference value, the sum of the points for STK17B, LOC93349 and CASP8 is a positive value.

7. The method according to claim 6, wherein the expression levels of AIM1, STK17B, LOC93349 and CASP8 are the expression levels of mRNAs encoding them, the relative expression level is the relative value to the expression level in COLO205 cell line, the reference value for AIM1 is 2.2, the upper reference value is 1.036 and the lower reference value is 0.514 for STK17B, the upper reference value is 0.666 and the lower reference value is 0.211 for LOC93349, and the upper reference value is 0.833 and the lower reference value is 0.519 for CASP8.

8. The method according to claim 19, wherein the sample is cancer cells, cancer tissue, blood, serum, blood plasma or urine.

9. The method according to claim 19, wherein the TRAIL signal activator is TRAIL, a TRAILR1 agonist compound, or a TRAILR2 agonist compound.

10. A method of diagnosing the sensitivity to a TRAIL signal activator in a patient suffering from cancer, the method comprising examining the expression or activity of STK17B, LOC93349 and CASP8 in a sample collected from the patient.

11. The method according to claim 10, wherein the method is a method of diagnosing the sensitivity to a TRAIL signal activator in a patient suffering from breast cancer or colon cancer, and the method comprises further examining the expression or activity of AIM1 in a sample collected from the patient.

12. The method according to claim 10, wherein the TRAIL signal activator is TRAIL, a TRAILR1 agonist compound or a TRAILR2 agonist compound.

13-15. (canceled)

16. A method of screening a compound having an anticancer effect, or a salt thereof, the method comprising employing the inhibition of AIM1 as an index.

17. (canceled)

18. A method of screening a compound having an anticancer effect, or a salt thereof, the method comprising employing the activation of STK17B, LOC93349, SP110, NOD27 or RHOBTB3 as an index.

19. A method of preventing or treating cancer, comprising administering an effective amount of a TRAIL signal activator to a TRAIL signal activator sensitive patient screened using, as an index, fluctuation in the expression or activity of a TRAIL signal activator sensitive marker in samples taken from test subjects.

20. A method of preventing or treating cancer, comprising administering an effective amount of an AIM1 inhibitor to a mammal.

21. A method of preventing or treating cancer, comprising administering an effective amount of an STK17B, LOC93349, SP110, NOD27 or RHOBTB3 activator to a mammal.

22-24. (canceled)


SEQUENCE LISTING (24 sequences)

SEQ ID NO: 1 - DNA, 7553 bp, Homo sapiens
  CDS (488)..(5656)
agatgccaccaccactgccaagcagctgcattcctcgccgggaaattcctccaggcaagagaacgcagagacgcccgcccgcagtccgggggaggacgcttcaccaggtgctggccacgaacaggaggctttcctgggtgtgaggggtgcgccagggtcgcccacccaggagcggcccgcgggaggactaggcgaggcccctaacggagcccccagtgtgtgtgccgaagaaggctccctggggccccgcaacgcccgcagccagccccccaagggcgcgtctgatttgccaggtgagcctccggccgagggcgcagcgcacacggccagctccgcgcaggcagactgcacagcccgccccaagggtcacgcccaccctgctaaggtgctaactttggacatctacttgagtaagactgagggggcacaagtggacgagccggtcgtgattactcccagagcggaagattgcggtgactgggacgacatggagaagaggtccagcggccgtaggtcggggaggcggagggggtcgcagaaatccaccgactcccccggcgcggacgccgagctccctgagagcgctgccagggacgacgcggtgttcgacgacgaggtggcgccaaacgcggccagcgataacgcctcggcggaaaagaaagtgaaatctccgcgggcagccctcgacgggggcgttgcctccgctgcgagcccagagtccaagcccagccccggtaccaaagggcagctccgaggggagtcggaccggagcaaacagccacccccggcttcgtcccccacgaagaggaagggcaggagccgtgccctcgaggccgtgcccgccccgcccgccagcggcccccgggctcccgccaaggagtccccacccaagagggtgcccgatcccagcccagtcaccaagggcactgcggccgagagcggggaggaggcggcgcgggccatcccccgcgagctcccggtcaagagcagctcgctgctgccggagatcaagcccgagcacaagaggggcccgctccccaaccacttcaacggccgggcagagggaggtcgaagcagagagctgggcagagcggccggagcgcctggagcttctgacgccgacggcttgaagcccaggaaccatttcggcgtgggcaggtcgacagtgaccactaaagtgaccctccctgccaagcccaaacatgtggaactaaatcttaaaacccctaagaatcttgacagtttgggaaatgagcacaatccatttagccagccagttcacaaaggcaacactgccaccaaaatctccttatttgaaaacaaacggacaaacagtagcccaagacacactgacattcgaggccaaaggaatactcctgcctctagtaaaacgtttgttgggagggcaaagctgaatttagccaaaaaagccaaagaaatggagcaacctgaaaagaaagtaatgccaaacagtccccagaatggtgtgctggttaaggaaactgctatagaaaccaaagttaccgtctcggaagaagagattctgccagcaaccagaggaatgaatggagactcttctgagaatcaagctcttggtcctcagcctaaccaagatgataaagcagatgtacaaacagatgctggctgcctttcagaaccagtggcttctgctctgattcctgtcaaggatcataagctcttagagaaggaggactcagaggctgcagacagcaaaagccttgtacttgaaaatgtaaccgatacagcacaagacatccccaccactgtggataccaaagatttacctccaacggccatgccaaagccacagcatacattttctgactcacagtcccctgctgagtcatctcctgggccttctctttcactgtctgcacccgctcctggggatgttcccaaagacacatgtgttcaatcacccataagcagtttcccatgcactgatctaaaagtgtcagaaaaccataaaggatgtgttttgcctgtgtctcgtcagaacaatgagaaaatgccacttttagaacttggaggagaaacaacccctcctttgtccacagagcgtagtccagaagctgtgggaagtgagtgtccatccagagtcctcgtccaggtcaggtccttcgtgctccccgtggagagcacccaggatgtgagctcccaggtcatcccagagagctctgaagttagagaagtgcagttgccaacttgtcacagtaatgaacctgaagtggtttccgttgcaagttgtgctcccccacaagaggaagtactgggcaatgaacactctcattgcacagcagagctcgcggcaaaatctggcccacaagtcataccgccagcatcagagaaaactctgcctattcaggctcaaagtcagggcagcagaacacccctgatggctgaatccagtcccaccaactctcccagcagcggaaatcacttagccactcctcaaaggccagatcagactgttacaaatggccaggatagccctgccagccttttgaacatttctgctggtagtgatgatagtgtatttgattcttcttctgatatggaaaaattcactgaaattataaaacagatggatagcgcagtttgtatgcccatgaaaagaaagaaggccaggatgccaaactctcctgctcctcactttgccatgcctcctattcacgaagaccatttagaaaaggtgtttgatcccaaagtgtttacctttggtttggggaagaagaaggaaagtcagccagaaatgtcaccggctttacatttgatgcagaaccttgacacaaaatccaaactgagacccaaacgtgcatctgctgaacagagcgtcctcttcaagtccctgcacaccaacactaatgggaacagtgagcctctggtgatgccggaaatcaatgacaaagagaacagggacgtcacaaatggtggcattaagagatcgagactagaaaaaagtgcacttttctcaagcttgttatcttctttaccacaagacaaaatcttttctccttctgtgacatcagtcaacactatgaccacggctttcagtacttctcagaacggttccctatctcagtcttcagtgtcacagcccacgactgagggtgccccgccctgtggtttgaacaaagaacagtcaaatcttctgcccgacaactccttaaaggtcttcaatttcaactcgtcaagtacatcacactccagtttgaaaagtccaagccacatggaaaaatacccgcaaaaagagaaaaccaaagaagatctggattcacgaagcaacctacacttgccagaaactaaattttctgaattgtcaaaactgaagaatgatgatatggaaaaggctaatcatattgaaagtgttattaaatcaaacttgccaaactgtgcaaacagtgacaccgacttcatgggtcttttcaaatcaagccggtatgacccaagcatttctttttctggaatgtcattatcagacacaatgacacttagaggaagtgtccaaaataaactcaatccccgacctggaaaggtagtgatatatagtgaacccgacgtctctgagaagtgcattgaagttttcagtgacattcaggattgcagttcttggagcctctctccagtgatactcataaaagttgttagaggatgttggattttgtatgagcaaccaaattttgaagggcactccatccccttagaagaaggagaattggaactctctggtctctggggtatagaagacattttggaaaggcacgaagaagcagagtctgataagccagtggtgattggttccatcagacatgtggttcaggattacagagttagtcacattgacttatttactgaaccagaagggttaggaatcctaagttcctactttgatgatactgaagaaatgcagggatttggtgtaatgcagaagacttgttccatgaaagtacattggggcacgtggctgatttatgaagaacctggatttcagggtgttcctttcatcctggaacctggtgaataccctgacttgtccttctgggatacagaagcagcgtacattggatccatgcggcctctgaaaatgggtggccgtaaagttgaattccctacagatccaaaggtagttgtttatgaaaagcctttctttgaaggaaaatgtgtggaactagaaacaggaatgtgtagttttgtcatggagggaggtgaaacagaagaggcgactggagacgatcatttgccgtttacgtcagtggggtctatgaaagttctaagaggcatttgggttgcatatgagaaacctggatttaccggtcatcagtatttgctagaagaaggagaatacagggactggaaagcctggggaggttacaatggagagcttcagtctttacgacctatattaggtgatttttcaaatgctcacatgataatgtacagtgaaaaaaactttggatccaaaggttccagtattgatgtattgggaattgttgctaatttaaaggagactggatatggagtgaagacacagtctattaatgtactgagtggagtatgggtagcctatgaaaatcctgacttcacaggagaacagtatatactggataaaggattttataccagttttgaggactggggaggcaaaaattgtaagatctcttctgttcaacctatatgtttggattctttcactggcccaaggagacgaaatcagattcacttgttttcagaaccacagtttcaaggtcacagtcaaagttttgaagaaacaacaagtcaaattgatgattcattttctaccaagtcttgcagagtttcaggaggcagctgggttgtatatgatggagaaaatttcactggtaatcaatacgtgttggaagaaggccattatccttgtctgtctgcaatgggatgcccgcctggagcaactttcaagtctcttcgttttatagatgttgaattttctgaaccaacaattattctctttgaaagagaagacttcaaaggaaaaaagattgaacttaatgcagaaactgtcaatctccgatccctgggattcaacacacaaatacgctctgttcaggttattggtggcatatgggttacttatgaatatggcagttacagagggcgacagttcctattgtcacctgcagaagtacctaattggtatgaattcagtggctgtcgccaaataggttctctacgaccttttgttcagaagcgaatttatttcagacttcgaaacaaagcaacagggttattcatgtcaaccaatggaaacttagaggatctgaagcttctgaggatacaggtcatggaggatgtcggggccgatgatcagatttggatctatcaagaaggatgtatcaaatgcaggatagcagaagactgctgcctgacgattgtgggcagcctggtaacatctggctccaagctaggcctggccctggaccagaatgctgacagccagttctggagcttgaagtccgatggcaggatttacagcaagttgaagccaaatttagttttagacattaaagggggcacacagtatgatcaaaatcacattatcctcaacactgtcagcaaagagaagtttacacaagtgtgggaagccatggtcctatatacctgaacaaagaaggaagaagaatcttctggaggtccttccagccaccttatttcttaaaaaggacaatgctgatggaagaccagactggaaagtggatcgactcctccttcattgattctaaattcaaccttaaatcatgctgccatgactcagagaacttactcatcgtttcaaaagactatcatagctttaaaccaataatttgtcctcctttcatttcttgcctttcatttttggtagctgcttaaacaggttgcctaattagcagcttttgggtgattttgtaaaatgttatatcaagatttcaagactgtgtacattttaaattatttccaaagatagtgacaggagagaactggaacaaatttaccaactttgtggacctacaaagcccttacactttaaagggtaagacaaaggcttaagtttgaaaggtagagaactgtttagcatctgagaagaaatactttattaggcctgtaattttggttcttggccttaaacactttctggaacctttaaatatgctgcatagcacaatgggaaagccttaggtattcacacatttaaggaactctaaacaaaatactattttcctttagttcatattaaaaattaatacattttaaaaatttaatgtcaaagtctggtaacatttgttagtaggatttgagttattattttttgagacaggatctcaggctggagtgcagtggcacaatcacggctcactgcagcctctacctccccaggctcaggtgatcctcccacctcagcctcccaagtagctgggactataggcacacatcaccaagcccagccaaattttgttttttttttgtagagatggggtttcatcacgttgcccaggctgatctcgaacctctgggctcaagcaattcactcgcctcggcctcccaaaatgctgggattacaggcctgagccactgcgcccagccaggatttgaattattttaactcatccatgggctgccctagaatgtcacaaatgagggttgtttaatgcctttcttatagctgctactggaacactattatgacctaatttatgagccatccttactcatctacaagtgctgaagcaatgttacatacttttttgctaaactcagattttttagcctaatttcttgtcctcctatccacctgcatccacacatggcctgcatggggctgccttccctgcagtgttctgcagccatgcttcagggtatagctgttggtggacagcctcaggtcttgggggcactatagccactaaacgaggtgtgaaaggctcaagaggatgaccagcaattaattatccccagaaagtgaaggaaaagagacctttagggatgttgctggtcaagtcttgatttgaccggagtcaaatcaatcttcaagcaatcttggaatcctcaactgcagtaagcatttcaaaatgcaaacaaactgcttaacaactgacaagacaccagcccatacgctgctcttccaacagtgggttctagctttgaacaaaagtgctaaacatttccttgaatatattcttcctctttttgtcctcatcactcaatactggtgctcttgtcacaggtagaacagcttgtttcttttccatctattcaagtgtgtttctaattctaaaatgctgatcttctctggagtctatggtaggcaattatggtcactggaatagtttgtcttgttttaaaatattattggtgcatgtacaacagcatccaacatatctgtcttgttcctagatatatagctctgattttaggccttttgtgcataccattacaatatggtggggtaagacattctacagtagcctgtgctgaactgatctcttaaataaacttgcttctggttaactatgttgatgaaa

SEQ ID NO: 2 - protein, 1723 aa, Homo sapiens
MEKRSSGRRSGRRRGSQKSTDSPGADAELPESAARDDAVFDDEVAPNAASDNASAEKKVKSPRAALDGGVASAASPESKPSPGTKGQLRGESDRSKQPPPASSPTKRKGRSRALEAVPAPPASGPRAPAKESPPKRVPDPSPVTKGTAAESGEEAARAIPRELPVKSSSLLPEIKPEHKRGPLPNHFNGRAEGGRSRELGRAAGAPGASDADGLKPRNHFGVGRSTVTTKVTLPAKPKHVELNLKTPKNLDSLGNEHNPFSQPVHKGNTATKISLFENKRTNSSPRHTDIRGQRNTPASSKTFVGRAKLNLAKKAKEMEQPEKKVMPNSPQNGVLVKETAIETKVTVSEEEILPATRGMNGDSSENQALGPQPNQDDKADVQTDAGCLSEPVASALIPVKDHKLLEKEDSEAADSKSLVLENVTDTAQDIPTTVDTKDLPPTAMPKPQHTFSDSQSPAESSPGPSLSLSAPAPGDVPKDTCVQSPISSFPCTDLKVSENHKGCVLPVSRQNNEKMPLLELGGETTPPLSTERSPEAVGSECPSRVLVQVRSFVLPVESTQDVSSQVIPESSEVREVQLPTCHSNEPEVVSVASCAPPQEEVLGNEHSHCTAELAAKSGPQVIPPASEKTLPIQAQSQGSRTPLMAESSPTNSPSSGNHLATPQRPDQTVTNGQDSPASLLNISAGSDDSVFDSSSDMEKFTEIIKQMDSAVCMPMKRKKARMPNSPAPHFAMPPIHEDHLEKVFDPKVFTFGLGKKKESQPEMSPALHLMQNLDTKSKLRPKRASAEQSVLFKSLHTNTNGNSEPLVMPEINDKENRDVTNGGIKRSRLEKSALFSSLLSSLPQDKIFSPSVTSVNTMTTAFSTSQNGSLSQSSVSQPTTEGAPPCGLNKEQSNLLPDNSLKVFNFNSSSTSHSSLKSPSHMEKYPQKEKTKEDLDSRSNLHLPETKFSELSKLKNDDMEKANHIESVIKSNLPNCANSDTDFMGLFKSSRYDPSISFSGMSLSDTMTLRGSVQNKLNPRPGKVVIYSEPDVSEKCIEVFSDIQDCSSWSLSPVILIKVVRGCWILYEQPNFEGHSIPLEEGELELSGLWGIEDILERHEEAESDKPVVIGSIRHVVQDYRVSHIDLFTEPEGLGILSSYFDDTEEMQGFGVMQKTCSMKVHWGTWLIYEEPGFQGVPFILEPGEYPDLSFWDTEAAYIGSMRPLKMGGRKVEFPTDPKVVVYEKPFFEGKCVELETGMCSFVMEGGETEEATGDDHLPFTSVGSMKVLRGIWVAYEKPGFTGHQYLLEEGEYRDWKAWGGYNGELQSLRPILGDFSNAHMIMYSEKNFGSKGSSIDVLGIVANLKETGYGVKTQSINVLSGVWVAYENPDFTGEQYILDKGFYTSFEDWGGKNCKISSVQPICLDSFTGPRRRNQIHLFSEPQFQGHSQSFEETTSQIDDSFSTKSCRVSGGSWVVYDGENFTGNQYVLEEGHYPCLSAMGCPPGATFKSLRFIDVEFSEPTIILFEREDFKGKKIELNAETVNLRSLGFNTQIRSVQVIGGIWVTYEYGSYRGRQFLLSPAEVPNWYEFSGCRQIGSLRPFVQKRIYFRLRNKATGLFMSTNGNLEDLKLLRIQVMEDVGADDQIWIYQEGCIKCRIAEDCCLTIVGSLVTSGSKLGLALDQNADSQFWSLKSDGRIYSKLKPNLVLDIKGGTQYDQNHIILNTVSKEKFTQVWEAMVLYT

SEQ ID NO: 3 - DNA, 1707 bp, Homo sapiens
  CDS (264)..(1379)
ctctccgctgctgtcgccaggagtcacttcacgagaagccaggtcacaaccgtcggcccttgtctggaaaagtaaaagtggatcctgccacgttcggagctccctggcgcctcgcccggctggagctagagaactcgtcctgtggcggcccccggcgtggggcgggacagcggccccctggagggggcagtcccgggagaacctgcggcggccggagcggtaaaaataagtgactaaagaagcagacctgggaatcacctaacatgtcgaggaggagatttgattgccgaagtatttcaggcctactaactacaactcctcaaattccaataaaaatggaaaactttaataatttctatatacttacatctaaagagctagggagaggtaaatttgctgtggttagacaatgtatatcaaaatctactggccaagaatatgctgcaaaatttctaaaaaagagaagaagaggacaggattgtcgggcagaaattttacacgagattgctgtgcttgaattggcaaagtcttgtccccgtgttattaatcttcatgaggtctatgaaaatacaagtgaaatcattttgatattggaatatgctgcaggtggagaaattttcagcctgtgtttacctgagttggctgaaatggtttctgaaaatgatgttatcagactcattaaacaaatacttgaaggagtttattatctacatcagaataacattgtacaccttgatttaaagccacagaatatattactgagcagcatataccctctcggggacattaaaatagtagattttggaatgtctcgaaaaatagggcatgcgtgtgaacttcgggaaatcatgggaacaccagaatatttagctccagaaatcctgaactatgatcccattaccacagcaacagatatgtggaatattggtataatagcatatatgttgttaactcacacatcaccatttgtgggagaagataatcaagaaacatacctcaatatctctcaagttaatgtagattattcggaagaaactttttcatcagtttcacagctggccacagactttattcagagccttttagtaaaaaatccagagaaaagaccaacagcagagatatgcctttctcattcttggctacagcagtgggactttgaaaacttgtttcaccctgaagaaacttccagttcctctcaaactcaggatcattctgtaaggtcctctgaagacaagacttctaaatcctcctgtaatggaacctgtggtgatagagaagacaaagagaatatcccagaggatagcagcatggtttccaaaagatttcgtttcgatgactcattacccaatccccatgaacttgtttcagatttgctctgttagcacttttttctttgactcatttggactgaatttgaaattttatatccactccagtgagattatgatttgtagcttcatatatgacatgtttatattgtaaatgcacttttccatggaataatttagggaagtgttttaatgttaaattactagttgctagcatgttatgatttcatatcctgagatagctctgcagataagaaaatatttaaatatatgacaaaaagtaaaattgtacatgtgagtttacatgttaatgaaataattcaacttcaaatgaacttaccagaatgttttgcatatcaacaaaaaaaaaaaaaaaaaa

SEQ ID NO: 4 - protein, 372 aa, Homo sapiens
MSRRRFDCRSISGLLTTTPQIPIKMENFNNFYILTSKELGRGKFAVVRQCISKSTGQEYAAKFLKKRRRGQDCRAEILHEIAVLELAKSCPRVINLHEVYENTSEIILILEYAAGGEIFSLCLPELAEMVSENDVIRLIKQILEGVYYLHQNNIVHLDLKPQNILLSSIYPLGDIKIVDFGMSRKIGHACELREIMGTPEYLAPEILNYDPITTATDMWNIGIIAYMLLTHTSPFVGEDNQETYLNISQVNVDYSEETFSSVSQLATDFIQSLLVKNPEKRPTAEICLSHSWLQQWDFENLFHPEETSSSSQTQDHSVRSSEDKTSKSSCNGTCGDREDKENIPEDSSMVSKRFRFDDSLPNPHELVSDLLC

SEQ ID NO: 5 - DNA, 2668 bp, Homo sapiens
  CDS (92)..(1831)
gggtcagggcagccacactgcacgcaggctgggccgactggggagctcataggccaggctctgacacccaggcagggcctagggtgggacgatggcaggtgggggcagcgacctgagcaccagggggctgaacggaggtgtttcacaagtagcaaatgagatgaaccatcttcctgcacacagccaaagtctgcaaaggctgttcacggaagaccaggatgtagatgagggacttgtctatgacactgtattcaagcacttcaaaagacataagctggagatatcaaatgcaataaaaaagacatttccattccttgagggcctccgcgatcgggaactcatcacaaataaaatgtttgaagattctgaagattcttgtagaaacctggtccctgtacaaagagtggtgtacaatgttctcagtgaactggagaagacatttaacctgtcagttttggaagcactgttcagcgaggtcaacatgcaggaataccccgatttaattcacatttataaaagcttcaaaaatgcaatccaagacaaattgtctttccaagaaagtgatcgaaaagaaagggaagagaggcctgacatcaaactaagtcttaaacaaggagaagtgccagaaagcccggaagcaaggaaggaaagtgaccaagcatgtggcaaaatggatactgtggatattgcaaacaactctactttgggaaaacccaagaggaaaagaagaaaaaagaaggggcatggctggagcagaatgggaacgagaacgcagaaaaacaaccaacaaaatgataacagcaaagccgatggccagctggtctcgagtgaaaagaaggcgaacatgaatctgaaagacctttccaagattagggggagaaagagaggcaaacctggaacccactttactcagagtgacagagctccacagaaaagagtccgatcaagagcttcaagaaagcacaaagatgaaactgtggattttcaggctcctttacttccagtgacctgtggtggggtgaagggaattttacataaggagaaattggaacaaggaaccttggcaaagtgtatacagactgaggatggaaaatggttcacccccatggaatttgaaatcaaaggaggctacgcaagatcaaagaactggaggctgagtgtgcgctgtggcgggtggcccctacgacggctgatggaggaaggatctctacctaatcctccaagaatatattacaggaacaaaaagagaatactgaagtctcaaaacaatagctcagttgacccttgtatgagaaacttggatgagtgtgaggtgtgccgggacggaggggagctgttctgttgcgacacttgttcaagagtcttccatgaggactgccacatcccacctgtggaaagtgagaagaccccgtggaattgcatcttctgcaggatgaaggagtctccgggaagccaacagtgttgtcaggaatctgaggtcctggagaggcagatgtgtcctgaggaacagttgaaatgtgagttcctcctcttgaaagtctattgctgttctgagagctccttttttgccaagattccatactattattatattagagaggcgtgtcaaggcctgaaggagcccatgtggttggataaaatcaagaaaaggctgaatgagcacggttacccccaagtggaggggtttgtacaagacatgcgcctcatcttccagaaccacagggcctcttacaagtataaggattttggccaaatgggacttagactggaggctgaatttgagaaggatttcaaggaagtgtttgctattcaggaaacaaatgggaacagttgactggtttagtggatgctgaaggccttcaggaaatatgctactggttgccactgacttcaaactgagagcacttgggaaatagcacatgcagggagaggcttttctctgagcctccctcatctgcccaaaaacaaatcctcaaaagaaatttgatcatcatgaatcccatccccaagaatctcatcaaccagggaagaggaactgagatcacagggaaggagattggaggtgataccagcacagacagactctcacctcttctcctgaggtctgctccagacaacatttattactcacaagacctttttcctccgttttttttttgagatggagtctcacacagtcacccgggattggaatgcaatggcgcgatctcagctcactgcaacctctgcctcccggattcaagtgattctcctgtctcagcctcccaagtagctgggattacagtgcctgccaccatgcccagctaattttttgcatttttagtagagacggggtttcactatgttggccaggctggtctcgaactcctgacctcatgagccgcccgccttggcctcccaaagtgctgggacgtgacaggagtgagccaccacacctggccactcgcaagaccttttatctgaaaaccagccaagctttattcacgacacacttcttcccttcactctcccacttctgtggtcaactccctgcagaactcccaaactgccgttcttttcgatagctcacgatggtgtatgagtgtcaatcatctgacccttcttggagtctcatatttcgtggaactcctgtgcaaacatatattattaaattttttttcctcctgtc

SEQ ID NO: 6 - protein, 580 aa, Homo sapiens
MAGGGSDLSTRGLNGGVSQVANEMNHLPAHSQSLQRLFTEDQDVDEGLVYDTVFKHFKRHKLEISNAIKKTFPFLEGLRDRELITNKMFEDSEDSCRNLVPVQRVVYNVLSELEKTFNLSVLEALFSEVNMQEYPDLIHIYKSFKNAIQDKLSFQESDRKEREERPDIKLSLKQGEVPESPEARKESDQACGKMDTVDIANNSTLGKPKRKRRKKKGHGWSRMGTRTQKNNQQNDNSKADGQLVSSEKKANMNLKDLSKIRGRKRGKPGTHFTQSDRAPQKRVRSRASRKHKDETVDFQAPLLPVTCGGVKGILHKEKLEQGTLAKCIQTEDGKWFTPMEFEIKGGYARSKNWRLSVRCGGWPLRRLMEEGSLPNPPRIYYRNKKRILKSQNNSSVDPCMRNLDECEVCRDGGELFCCDTCSRVFHEDCHIPPVESEKTPWNCIFCRMKESPGSQQCCQESEVLERQMCPEEQLKCEFLLLKVYCCSESSFFAKIPYYYYIREACQGLKEPMWLDKIKKRLNEHGYPQVEGFVQDMRLIFQNHRASYKYKDFGQMGLRLEAEFEKDFKEVFAIQETNGNS

SEQ ID NO: 7 - DNA, 2914 bp, Homo sapiens
  CDS (304)..(1791)
gtgctctgagtttttggtttctgtttcaccttgtgtctgagctggtctgaaggctggttgttcagactgagcttcctgcctgcctgtaccccgccaacagcttcagaagaaggtgactggtggctgcctgaggaataccagtgggcaagagaattagcatttctggagcatctgctgtctgagcagcccctgggtgcgtccactttctgggcacgtgaggttgggccttggccgcctgagcccttgagttggtcacttgaaccttgggaatattgagattatattctcctgccttttaaaaagatggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacaggttccacttctgccgcatgagctgggctgaagcaaacagccagtgccagacacagtctgtacctttctggcggagggtcgatcatctattaataagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaaggggaggagttgtgtggggtaatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattgatggtgctattttgtttgttttgttttgttttgtttttttgagacagaatctcgctctgtcgcccaggctggagtgcagtggcgtgatctcggctcaccgcaagctccgcctcccgggttcaggccattctcctgcctcagcctcccgagtagctgggactacaggggcccgccaccacacctggctaattttttaaaaatatttttagtagagacagggtttcactgtgttagccagggtggtcttgatctcctgacctcgtgatccacccacctcggcctcccaaagtgctgggattacaggcgtgagccaccgcgcctggccgatggtactatttagatataacactatgtttatttactaattttctagattttctactttattaattgttttgcacttttttataagagctaaagttaaataggatattaacaacaataacactgtctcctttctcttatgcttaaggctttgggaatgtttttagctggtggcaataaataccagacacgtacaaaatccagctatgaatatagagggcttatgattcagattgttatctatcaactataagcccactgttaatattctattaactttaattctctttcaaagctaaattccacactaccacattaaaaaaattagaaagtagccacgtatggtggctcatgtctataatcccagcactttgggaggttgaggtgggaggattgcttgaacccaagaggtcaaggctgcagtgagccatgttcacaccgctgcactcaagcttgggtgacagaacaagaccccgtctcaaaaaaaattttttttttaataaaacaaaatttgtttgaaatcttttaaaaattcaaatgatttttacaagttttaaataagctctccccaaacttgctttatgccttcttattgcttttatgatatatatatgcttggctaactatatttgctttttgctaacaatgctctggggtctttttatgcatttgcatttgctctttcatctctgcttggattattttaaatcattaggaattaagttatctttaaaatttaagtatcttttttcaaaaacattttttaatagaataaaatataatttgatcttattaaa

SEQ ID NO: 8 - protein, 496 aa, Homo sapiens
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRFHFCRMSWAEANSQCQTQSVPFWRRVDHLLIRVMLYQISEEVSRSELRSFKFLLQEEISKCKLDDDMNLLDIFIEMEKRVILGEGKLDILKRVCAQINKSLLKIINDYEEFSKGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 9 - DNA, 2431 bp, Homo sapiens
  CDS (102)..(2240)
gttttgcctgctagcatctccctgtaactctcccaatcttgaggagtgatccctgtcccagcccctggaaaggggcaggaacgacaaactcaaagtccaggatgttcaccatgacaagagccatggaagaggctctttttcagcacttcatgcaccagaagctggggatcgcctatgccatacacaagccatttcccttctttgaaggcctcctagacaactccatcatcactaagagaatgtacatggaatctctggaagcctgtagaaatttgatccctgtatccagagtggtgcacaacattctcacccaactggagaggacttttaacctgtctcttctggtgacattgttcagtcaaattaacctgcgtgaatatcccaatctggtgacgatttacagaagcttcaaacgtgttggtgcttcctatgaacggcagagcagagacacaccaatcctacttgaagccccaactggcctagcagaaggaagctccctccataccccactggcgctgcccccaccacaaccccctcaaccaagctgttcaccctgtgcgccaagagtcagtgagcctggaacatcctcccagcaaagcgatgagatcctgagtgagtcgcccagcccatctgaccctgtcctgcctctccctgcactcatccaggaaggaagaagcacttcagtgaccaatgacaagttaacatccaaaatgaatgcggaagaagactcagaagagatgcccagcctcctcactagcactgtgcaagtggccagtgacaacctgatcccccaaataagagataaagaagaccctcaagagatgccccactctcccttgggctctatgccagagataagagataattctccagaaccaaatgacccagaagagccccaggaggtgtccagcacaccttcagacaagaaaggaaagaaaagaaaaagatgtatctggtcaactccaaaaaggagacataagaaaaaaagcctcccaagagggacagcctcatctagacacggaatccaaaagaagctcaaaagggtggatcaggttcctcaaaagaaagatgactcaacttgtaactccacggtagagacaagggcccaaaaggcgagaactgaatgtgcccgaaagtcgagatcagaggagatcattgatggcacttcagaaatgaatgaaggaaagaggtcccagaagacgcctagtacaccacgaagggtcacacaaggggcagcctcacctgggcatggcatccaagagaagctccaagtggtggataaggtgactcaaaggaaagacgactcaacctggaactcagaggtcatgatgagggtccaaaaggcaagaactaaatgtgcccgaaagtccagatcgaaagaaaagaaaaaggagaaagatatctgttcaagctcaaaaaggagatttcagaaaaatattcaccgaagaggaaaacccaaaagtgacactgtggattttcactgttctaagctccccgtgacctgtggtgaggcgaaagggattttatataagaagaaaatgaaacacggatcctcagtgaagtgcattcggaatgaggatggaacttggttaacaccaaatgaatttgaagtcgaaggaaaaggaaggaacgcaaagaactggaaacggaatatacgttgtgaaggaatgaccctaggagagctgctgaagcggaaaaactcggatgaatgcgaggtgtgctgtcaagggggacaacttctctgctgcggtacttgtccacgagtcttccatgaggactgtcacatcccccctgtggaagccaagaggatgctgtggagttgcaccttctgcaggatgaagaggtcttcaggaagccaacagtgccatcatgtatctaagaccctggagaggcagatgcagcctcaggaccagctgaaatgtgagttcctcctcttgaaggcctactgtcatccacaaagctccttttttacgggcatcccatttaatattcgagattacggtgagccctttcaggaagcaatgtggttggacctggttaaggaaaggctgattacggaaatgtacacggtggcatggtttgtgcgagacatgcgcctgatgtttcgcaaccataaaacattttacaaggcttctgactttggccaggtaggacttgacttagaggcagaatttgaaaaagatctcaaagacgtgctcggttttcatgaagccaatgacggcggtttctggactcttccttgaccctgttctgtaaagactgaagcatccccacctcaggattcagctgatgggaccctggcttggactgttgattgccagtgagtctgggatgtaattggctgccctcaggacccaaacccagacacttcataggattatcacaccctccatctttattctttctttttacctttaaaagtctatatcta

SEQ ID NO: 10 - protein, 713 aa, Homo sapiens
MFTMTRAMEEALFQHFMHQKLGIAYAIHKPFPFFEGLLDNSIITKRMYMESLEACRNLIPVSRVVHNILTQLERTFNLSLLVTLFSQINLREYPNLVTIYRSFKRVGASYERQSRDTPILLEAPTGLAEGSSLHTPLALPPPQPPQPSCSPCAPRVSEPGTSSQQSDEILSESPSPSDPVLPLPALIQEGRSTSVTNDKLTSKMNAEEDSEEMPSLLTSTVQVASDNLIPQIRDKEDPQEMPHSPLGSMPEIRDNSPEPNDPEEPQEVSSTPSDKKGKKRKRCIWSTPKRRHKKKSLPRGTASSRHGIQKKLKRVDQVPQKKDDSTCNSTVETRAQKARTECARKSRSEEIIDGTSEMNEGKRSQKTPSTPRRVTQGAASPGHGIQEKLQVVDKVTQRKDDSTWNSEVMMRVQKARTKCARKSRSKEKKKEKDICSSSKRRFQKNIHRRGKPKSDTVDFHCSKLPVTCGEAKGILYKKKMKHGSSVKCIRNEDGTWLTPNEFEVEGKGRNAKNWKRNIRCEGMTLGELLKRKNSDECEVCCQGGQLLCCGTCPRVFHEDCHIPPVEAKRMLWSCTFCRMKRSSGSQQCHHVSKTLERQMQPQDQLKCEFLLLKAYCHPQSSFFTGIPFNIRDYGEPFQEAMWLDLVKERLITEMYTVAWFVRDMRLMFRNHKTFYKASDFGQVGLDLEAEFEKDLKDVLGFHEANDGGFWTLP

SEQ ID NO: 11 - DNA, 6822 bp, Homo sapiens
  CDS (226)..(5823)
gcagcagccgccagttccatgcactggcggagcagctctaggcggcggcttctactttcagtttcgtgcagagcgcggaggagccgcgagcgctgagggagtctgcactatggaaacaacctgtcaatccagctcaaggcacacatagcccagacacccatgagaccctctccgtggggaccctagagcacctatcatgaacgaggagaccaaggctggctcctcatggaccccgttggcctccagctcggcaacaagaacctgtggagctgtcttgtgaggctgctcaccaaagacccagaatggctgaacgccaagatgaagttcttcctccccaacacggacctggattccaggaacgagaccttggaccctgaacagagagtcatcctgcaactcaacaagctgcatgtccagggttcggacacctggcagtctttcattcattgtgtgtgcatgcagctggaggtgcctctggacctggaggtgctgctgctgagtacttttggctatgatgatgggttcaccagccagctgggagctgaggggaaaagccaacctgaatctcagctccaccatggcctgaagcgcccacatcagagctgtgggtcctcaccccgccggaagcagtgcaagaagcagcagctagagttggccaagaagtacctgcagctcctgcggacctctgcccagcagcgctacaggagccaaatccctgggtcagggcagccccacgccttccaccaggtctatgtccctccaatcctgcgccgggccacagcatccttagacactccggagggggccattatgggggacgtcaaggtggaagatggtgctgacgtgagcatctcggacctcttcaacaccagggttaacaagggcccgagggtgaccgtgcttttggggaaggctggcatgggcaagaccacgctggcccaccggctctgccagaagtgggcagagggccatctgaactgtttccaggccctgttcctttttgaattccgccagctcaacttgatcacgaggttcctgacaccgtccgagctcctttttgatctgtacctgagccctgaatcggaccacgacactgtcttccagtacctggagaagaacgctgaccaagtcctgctgatctttgatgggctagatgaggccctccagcctatgggtcctgatggcccaggcccagtcctcacccttttctcccatctctgcaatgggaccctcctgcctggctgccgggtgatggctacctcccgtccagggaagctgcctgcctgcctgcctgcagaggcagccatggtccacatgttgggctttgatgggccacgggtggaagaatatgtgaatcacttcttcagcgcccagccatcgcgggagggggccctggtggagttacagacaaatggacgtctccgaagcctgtgtgcggtgcccgcactgtgccaagtcgcctgtctctgcctccaccatctgcttcctgaccacgccccaggccagtctgtggccctcctgcccaacatgactcagctctatatgcagatggtgctcgccctcagcccccctgggcacttgcccacctcgtccctactggacctgggggaggtggccctgaggggcctggagacagggaaggttatcttctatgcaaaagatattgctccacccttgatagcttttggggccactcacagcctgctgacttccttctgcgtctgcacaggccctgggcaccagcagacaggctatgctttcacccacctcagcctgcaggagtttcttgctgccctgcacctgatggccagccccaaggtgaacaaagacacacttacccagtatgttaccctccattcccgctgggtacagcggaccaaagctagactgggcctctcagaccacctccccaccttcctggcgggcctggcatcctgcacctgccgccccttccttagccacctggcgcagggcaatgaggactgtgtgggtgccaagcaggctgctgtagtgcaggtgttgaagaagttggccacccgcaagctcacagggccaaaggttgtagagctgtgtcactgtgtggatgagacacaggagcctgagctggccagtctcaccgcacaaagcctcccctatcaactgcccttccacaatttcccactgacctgcaccgacctggccaccctgaccaacatcctagagcacagggaggcccccatccacctggattttgatggctgtcccctggagccccactgccctgaggctctggtaggctgtgggcagatagagaatctcagctttaagagcaggaagtgtggggatgcctttgcagaagccctctccaggagcttgccgacaatggggaggctgcagatgctggggttagcaggaagtaaaatcactgcccgaggcatcagccacctggtgaaagctttgcctctctgtccacagctgaaagaagtcagttttcgggacaaccagctcagtgaccaggtggtgctgaacattgtggaggttctccctcacctaccacggctccggaagcttgacctgagcagcaacagcatctgcgtgtcaaccctactctgcttggcaagggtggcagtcacgtgtcctaccgtcaggatgcttcaggccagggaggcggacctcatcttccttctttccccgcccacagagacaactgcagagctacaaagagctccagacctgcaggaaagtgacggccagaggaaaggggctcagagcagaagcttgacgctcaggctgcagaagtgtcagctccaggtccacgatgcggaggccctcatagccctgctccaggaaggccctcacctggaggaagtggacctctcagggaaccagctggaagatgaaggctgtcggctgatggcagaggctgcatcccagctgcacatcgccaggaagctggacctcagtgacaacgggctttctgtggccggggtgcattgtgtgctgagggccgtgagtgcgtgctggaccctggcagagctgcacatcagcctgcagcacaaaactgtgatcttcatgtttgcccaggagccagaggagcagaaggggccccaggagagggctgcatttcttgacagcctcatgctccagatgccctctgagctgcctctgagctcccgaaggatgaggctgacacattgtggcctccaagaaaagcacctagagcagctctgcaaggctctgggaggaagctgccacctcggtcacctccacctcgacttctcaggcaatgctctgggggatgaaggtgcagcccggctggctcagctgctcccagggctgggagctctgcagtccttgaacctcagtgagaacggtttgtccctggatgccgtgttgggtttggttcggtgcttctccactctgcagtggctcttccgcttggacatcagctttgaaagccaacacatcctcctgagaggggacaagacaagcagggatatgtgggccactggatctttgccagacttcccagctgcagccaagttcttagggttccgtcagcgctgcatccccaggagcctctgcctcagtgagtgtcctctggagcccccaagcctcacccgcctctgtgccactctgaaggactgcccgggacccctggaactgcaattgtcctgtgagttcctgagtgaccagagcctggagactctactggactgcttacctcaactccctcagctgagcctgctgcagctgagccagacgggactgtccccgaaaagccccttcctgctggccaacaccttaagcctgtgtccacgggttaaaaaggtggatctcaggtccctgcaccatgcaactttgcacttcagatccaacgaggaggaggaaggcgtgtgctgtggcaggttcacaggctgcagcctcagccaggagcacgtagagtcactctgctggttgctgagcaagtgtaaagacctcagccaggtggatctctcagcaaacctgctgggcgacagcggactcagatgccttctggaatgtctgccgcaggtgcccatctccggtttgcttgatctgagtcacaacagcatttctcaggaaagtgccctgtacctgctggagacactgccctcctgcccacgtgtccgggaggcctcagtgaacctgggctctgagcagagcttccggattcacttctccagagaggaccaggctgggaagacactcaggctaagtgagtgcagcttccggccagagcacgtgtccaggctggccaccggcttgagcaagtccctgcagctgacggagctcacgctgacccagtgctgcctgggccagaagcagctggccatcctcctgagcttggtggggcgacccgcagggctgttcagcctcagggtgcaggagccgtgggcggacagagccagggttctctccctgttagaagtctgcgcccaggcctcaggcagtgtcactgaaatcagcatctccgagacccagcagcagctctgtgtccagctggaatttcctcgccaggaagagaatccagaagctgtggcactcaggttggctcactgtgaccttggagcccaccacagccttcttgtcgggcagctgatggagacatgtgccaggctgcagcagctcagcttgtctcaggttaacctctgtgaggacgatgatgccagttccctgctgctgcagagcctcctgctgtccctctctgagctgaagacatttcggctgacctccagctgtgtgagcaccgagggcctcgcccacctggcatctggtctgggccactgccaccacttggaggagctggacttgtctaacaatcaatttgatgaggagggcaccaaggcgctgatgagggcccttgaggggaaatggatgctaaagaggctggacctcagtcaccttctgctgaacagctccaccttggccttgcttactcacagactaagccagatgacctgcctgcagagcctcagactgaacaggaacagtatcggtgatgtcggttgctgccacctttctgaggctctcagggctgccaccagcctagaggagctggacttgagccacaaccagattggagacgctggtgtccagcacttagctaccatcctgcctgggctgccagagctcaggaagatagacctctcagggaatagcatcagctcagccgggggagtgcagttggcagagtctctcgttctttgcaggcgcctggaggagttgatgcttggctgcaatgccctgggggatcccacagccctggggctggctcaggagctgccccagcacctgagggtcctacacctaccattcagccatctgggcccaggtggggccctgagcctggcccaggccctggatggatccccccatttggaagagatcagcttggcggaaaacaacctggctggaggggtcctgcgtttctgtatggagctcccgctgctcagacagatagacctggtttcctgtaagattgacaaccagactgccaagctcctcacctccagcttcacgagctgccctgccctggaagtaatcttgctgtcctggaatctcctcggggatgaggcagctgccgagctggcccaggtgctgccgcagatgggccggctgaagagagtggacctggagaagaatcagatcacagctttgggggcctggctcctggctgaaggactggcccaggggtctagcatccaagtcatccgcctctggaataaccccattccctgcgacatggcccagcacctgaagagccaggagcccaggctggactttgccttctttgacaaccagccccaggccccttggggtacttgatggccccctcaagacctttggaatccagccaagtgatgcacccaaatgatccacctttcgcccactgggataattgactcaggaaagaagagcctcggcagggcgctctgcactccacccaggaggaaggatacgtgtgtcctgctgcagtcctcagggagaacttttttgggaaccaggagctgggtctggacaaaggagtaccctgcattacgtgggatatgtgtgatcaattggggacatgcgacacacaatgagggtgtcatgacaatgcatgacacgtacggttatatgtggcagtgtgaccccttgacatgtggcgttacatgaaagtcagtgtggcacgtgttctgtggcatgggtgctggcatcccaagtagcaggatacatgattgttggtctatatatgacacatgacaaatgtccatgtcacaggactcatggctggccagatgacctcaggctggcccaagatctaatttattaatttttaaagcaaatacatatttatagattgtgtgtatggagcagctaagtcaggaaaagtcttccgcccgagctgggaggggagagtgtccatgcactgaccagtccaggggctcaagggccagggctctggaacaagccagggactcagccattaagtcccctcctgcctcaatcctcagcctacccatctataaacttgatgactcctcccttacttacatactagcttccaaggacaggtggaggtagggccagcctggcgggagtggagaagcccagtctgtcctatgtaagggacaaagccaggtctaatggtactgggtagggggcactgccaagacaataagctaggctactgggtccagctactactttggtgggattcaggtgagtctccatgcacttcacatgttacccagtgttcttgttacttccaaggagaaccaagaatggctctgtcacactcgaagccaggcttgatcaataaacacaatggtattccacgtc

SEQ ID NO: 12 - protein, 1866 aa, Homo sapiens
MDPVGLQLGNKNLWSCLVRLLTKDPEWLNAKMKFFLPNTDLDSRNETLDPEQRVILQLNKLHVQGSDTWQSFIHCVCMQLEVPLDLEVLLLSTFGYDDGFTSQLGAEGKSQPESQLHHGLKRPHQSCGSSPRRKQCKKQQLELAKKYLQLLRTSAQQRYRSQIPGSGQPHAFHQVYVPPILRRATASLDTPEGAIMGDVKVEDGADVSISDLFNTRVNKGPRVTVLLGKAGMGKTTLAHRLCQKWAEGHLNCFQALFLFEFRQLNLITRFLTPSELLFDLYLSPESDHDTVFQYLEKNADQVLLIFDGLDEALQPMGPDGPGPVLTLFSHLCNGTLLPGCRVMATSRPGKLPACLPAEAAMVHMLGFDGPRVEEYVNHFFSAQPSREGALVELQTNGRLRSLCAVPALCQVACLCLHHLLPDHAPGQSVALLPNMTQLYMQMVLALSPPGHLPTSSLLDLGEVALRGLETGKVIFYAKDIAPPLIAFGATHSLLTSFCVCTGPGHQQTGYAFTHLSLQEFLAALHLMASPKVNKDTLTQYVTLHSRWVQRTKARLGLSDHLPTFLAGLASCTCRPFLSHLAQGNEDCVGAKQAAVVQVLKKLATRKLTGPKVVELCHCVDETQEPELASLTAQSLPYQLPFHNFPLTCTDLATLTNILEHREAPIHLDFDGCPLEPHCPEALVGCGQIENLSFKSRKCGDAFAEALSRSLPTMGRLQMLGLAGSKITARGISHLVKALPLCPQLKEVSFRDNQLSDQVVLNIVEVLPHLPRLRKLDLSSNSICVSTLLCLARVAVTCPTVRMLQAREADLIFLLSPPTETTAELQRAPDLQESDGQRKGAQSRSLTLRLQKCQLQVHDAEALIALLQEGPHLEEVDLSGNQLEDEGCRLMAEAASQLHIARKLDLSDNGLSVAGVHCVLRAVSACWTLAELHISLQHKTVIFMFAQEPEEQKGPQERAAFLDSLMLQMPSELPLSSRRMRLTHCGLQEKHLEQLCKALGGSCHLGHLHLDFSGNALGDEGAARLAQLLPGLGALQSLNLSENGLSLDAVLGLVRCFSTLQWLFRLDISFESQHILLRGDKTSRDMWATGSLPDFPAAAKFLGFRQRCIPRSLCLSECPLEPPSLTRLCATLKDCPGPLELQLSCEFLSDQSLETLLDCLPQLPQLSLLQLSQTGLSPKSPFLLANTLSLCPRVKKVDLRSLHHATLHFRSNEEEEGVCCGRFTGCSLSQEHVESLCWLLSKCKDLSQVDLSANLLGDSGLRCLLECLPQVPISGLLDLSHNSISQESALYLLETLPSCPRVREASVNLGSEQSFRIHFSREDQAGKTLRLSECSFRPEHVSRLATGLSKSLQLTELTLTQCCLGQKQLAILLSLVGRPAGLFSLRVQEPWADRARVLSLLEVCAQASGSVTEISISETQQQLCVQLEFPRQEENPEAVALRLAHCDLGAHHSLLVGQLMETCARLQQLSLSQVNLCEDDDASSLLLQSLLLSLSELKTFRLTSSCVSTEGLAHLASGLGHCHHLEELDLSNNQFDEEGTKALMRALEGKWMLKRLDLSHLLLNSSTLALLTHRLSQMTCLQSLRLNRNSIGDVGCCHLSEALRAATSLEELDLSHNQIGDAGVQHLATILPGLPELRKIDLSGNSISSAGGVQLAESLVLCRRLEELMLGCNALGDPTALGLAQELPQHLRVLHLPFSHLGPGGALSLAQALDGSPHLEEISLAENNLAGGVLRFCMELPLLRQIDLVSCKIDNQTAKLLTSSFTSCPALEVILLSWNLLGDEAAAELAQVLPQMGRLKRVDLEKNQITALGAWLLAEGLAQGSSIQVIRLWNNPIPCDMAQHLKSQEPRLDFAFFDNQPQAPWGT

SEQ ID NO: 13 - DNA, 5566 bp, Homo sapiens
  CDS (538)..(2370)
cgcgcgggcggctcctttgtgtccagccgccgccaccggagctcccggggcctccgcggggagcgcgtcccccgcatccgcccgacccccggggctggcacgtgctgcgcccggtccgctgagggggcggaggccccgatctccccgaccccccttctctgcttagaggaggaggagcagcggcagcggcagcaggaggcgacagctgccagccgaggaggcgcggcggagaggggactgcggtcagctgcgtccacttggggctgtgcggcggtcccgcgcccggcgatgttcccgggcactccctgagtagcggcagcttatcccccgcccgctagcccgccctggtccccggctcgctcgctggctggcgcggccccggccccgctctgcgtcggccccgccgcggtggaggcgcgcgagggggacgcggccggggatgagcggattgcgggtgaactcgccgcccgggggccccgcgaagccgtgagccgctgcttttctccgagtcgccgccctgcccttggatttgagatcatgtccatccacatcgtggcgctggggaacgagggggacacattccaccaggacaaccggccgtcggggcttatccgcacttacctggggagaagccctctggtctccggggacgagagcagcttgttgctgaacgcggccagcacggtcgcgcgtccggtgttcaccgagtatcaggccagtgcgtttgggaatgtcaagctggtggtccacgactgtcccgtctgggacatatttgacagtgattggtacacttctcgaaatctaattgggggcgctgacatcattgtgatcaaatacaacgttaatgacaagttttcattccatgaagtaaaggataattatattccagtgataaaaagagcattaaattcagttccagtaattattgctgctgttggtaccagacaaaatgaagagttaccttgtacatgcccactatgtacctcagacagagggagctgtgttagtacaactgaagggatccaacttgcaaaagaactaggagcgacctatcttgaactccacagccttgatgacttctacataggaaagtattttggaggagtgttggagtattttatgattcaagccttaaatcagaagacaagtgaaaaaatgaagaaaagaaaaatgagcaactcctttcatggaattagaccacctcaacttgaacaaccagaaaaaatgcctgtcttaaaggctgaagcgtcacattataactctgacttaaataacttgctgttctgctgccagtgtgtggacgtggtattttacaaccccaatttaaagaaagttgtagaggcccacaagatcgttctctgcgctgtaagccatgttttcatgctgcttttcaatgtgaagagtcccactgacattcaggattccagtatcatccgaactacccaggatctttttgctataaacagagatactgcatttccaggtgctagccatgaatcttcaggcaacccaccattacgagtcattgttaaagacgccctcttctgttcttgtttatcagacatccttcgcttcatttattcaggtgcttttcagtgggaagaattggaagaagatatcaggaagaagttgaaagattctggggatgtttcaaatgtaatcgagaaagttaaatgcattttaaaaacaccaggaaagattaattgcctaaggaattgcaaaacctatcaagccagaaaacctttgtggttttataacacttccctcaagtttttccttaataagccgatgcttgccgatgttgtcttcgaaattcaaggtacgacagtgccagcccacagggccatcctggtggcccgttgtgaagtgatggcagccatgtttaatggtaattacatggaagcaaagagtgtcctgattcccgtttatggtgtttccaaagagactttcttgtcatttttagaatacctgtacacagactcctgctgcccagctggcatattccaggccatgtgtctcctgatctgtgccgagatgtaccaagtgtccagactgcagcacatctgtgagctgttcatcattacccagctgcagagcatgccaagcagggaactggcatccatgaaccttgatatagttgacctgcttaaaaaggccaagtttcaccactctgattgcctttcaacctggctacttcatttcattgctactaactacctcatcttcagtcaaaagcctgaatttcaggatctttcagtggaagaacgcagttttgttgaaaagcacagatggccgtcgaatatgtacttgaagcagcttgcggaatacaggaagtatattcactcccggaaatgtcgttgcttagtaatgtaacctggagcttttatacactacatttcttttttattattatgaagaatgggatacctccaggttccagtaaaattcttctgaccgaaaccaatgtgggtgttagaaaaattaccatatagcttaatatgtttattagttctctttggaaaaaaactaccactgtggtcttaaaagggaacaaaatataccataggctaaaactaaggctttcactctagaatgcaaagctgttttgcagctgttttcccttaaagatgtcctgttgctttagtgatatttagacccctctcagttaagaaatgcttagattaaaaaaaaaaaattacgtaggattaatacagaaatttaatcatgtctgattaattgctctattaaaataaggggcatttaaagacccagcataaccatttgtataatgagaaatctaggggaaaaccaatcagtccaacatgagattttaggaatagaaatttgccggccatttggaaagtgaaatgccacttagttctcaattgatgacagtgtttgaatcatcataaaaaaaatacctgcttttcatctggacaacccaattgagccactttatctccttttggcaatctgagtaggcggggaacctaggcagggctggctttcttagcgtgtaacttgtgtagcagcacagggcccacacttagaaggaccccacacttggttcaaggctctgctatagcggaaattcttaataatgtttgaagaagggccccatgatttcattttgtgctgagccctcaaaattatgtctgtttcgtggtgggaaatatcctatgttttcttgctcaaacacctttctctctgaaagcagaaaaaggcactgatataaagggaagagaaggaggctcaccggagggaagagaacatagtgaagattcccgcctttggggaggtctggaccacccagggcctccactgccaccttggctggcaagggagaaatgtgttgtgttgtcttagctttaaaacagtcacagttcttgctctatcatagatgaacaaatactttcttgatcattctgtaagaccaggaggttggtaagagtgactaaccagcctaactttaatacacatgtataaagatgttcacagagaaagatgctctgtagagaatttgctaccgaagttggctcaagaatttgtttttagtgttatttaccaagattaggacgtcagtggcttaaattctttgaattcttttcaaggactgcaagattatttgataaagagtagcatgaatcttgtgctctaatattacacagtaagttcaaagaaaggatgtaagtcaaagacttgttacatagagggaaaatggactgggatagaggacagactgatagtttctttctttcatatcacatgtatagagaaataattatatcagaaactcacaaacctagacatggaaaaacagattactgtctattgtcagcatcattttcatctgtaagtcactactggaatatatttttcttttaatttccagtgactttagaatacacacagtttttccgacttttcaaaaatttgattaaatggttttatagtataatattgggaccccataccgttagcccttgtatgtataccaacactgccaaagtaaaacattaggtcaggcatggtggctcaggcctgtaatcccagcattttgggaggctgaggcaagtggataacttgaggtcatgagttcgaaaccagcctggccaaaacagtgaaaccccgtctctactaaaaatacaaaattagccagatgtggtggcgcacacctgtaatcccagctactcaggaagctgaggcaggaaaatcgcttgaacctgggaggtggaagttgcagtgagccgagatcgcaccactgcactccagcctgggtgacaagagcgaaactccatctcaaaaaaaaaaaaaaaaccaaagtgaaacactaaaaattccctatagatatattccaggaaatattttaattgggctgattttaattaggctgtatcattgatgattactggaatcgattttatgtcttttgtattttaatcacttgagttaatcaaccactggcaaatcccatttgacaaagattagcattgtaaaaaacagatactgtggtagatttctagaaattcattcacatttaagacttctaaaatggaataatagccttttgtttttcatgagcatattcgcacccctatatgaattacagcatttaaagttcaaaatcagtaacttttaatctaggaaattgaaaaatattaagttgcaaagcaaaaaaaggtattttcttgaaaatactatttaatgtttaactagactataggtagttccttaaggttgtttgacctgaagtggagttgggtttggaagctggtgcccagttggtgtggagtgtgtagttttgttatgaaagttctctaccacctacctgtgtgagtgacaccaacatccagatgtcacagctctccagagctagtcagaagagaaatcaaattagtgtttaaacccatttgcatattgacttgtcagtacctttaactcaatttaatataacaagaaatcgtaaaatacttataacctatcttagagaaatgagtgctggttttgagagttgttttttaactgaaagattatttctagatgggtagtgctttgtgctggtttctgcttccatatatttcccagtcattttaattagagaagatactctatggtagaactaaggcctttcctttcttggccaaagtctttaccctatttaaccctttgtatatttctgactgctcactgttcatattataggggaccagatttgtaatatagaattctccataacatgaatgaaattaattctgtccaagccagcatggtggcttcatattaagtagtaacagaagtctgaacaattggataaatttgacttccaagacagctaaacttttcaactgcaattttaaaaactacactacactgttatagttaatctgacaaaaatgtcctcaaagagtactttattttatttaaagcatctgtttaattcaacctttaataattttgcaaagaagggtatgtgtgtattttaatatagcctgacctgaatttatatgtttttagctttagtatttaactttttgtaacaaataaaccttttttaaaacaagtttaa

SEQ ID NO: 14 - protein, 611 aa, Homo sapiens
MSIHIVALGNEGDTFHQDNRPSGLIRTYLGRSPLVSGDESSLLLNAASTVARPVFTEYQASAFGNVKLVVHDCPVWDIFDSDWYTSRNLIGGADIIVIKYNVNDKFSFHEVKDNYIPVIKRALNSVPVIIAAVGTRQNEELPCTCPLCTSDRGSCVSTTEGIQLAKELGATYLELHSLDDFYIGKYFGGVLEYFMIQALNQKTSEKMKKRKMSNSFHGIRPPQLEQPEKMPVLKAEASHYNSDLNNLLFCCQCVDVVFYNPNLKKVVEAHKIVLCAVSHVFMLLFNVKSPTDIQDSSIIRTTQDLFAINRDTAFPGASHESSGNPPLRVIVKDALFCSCLSDILRFIYSGAFQWEELEEDIRKKLKDSGDVSNVIEKVKCILKTPGKINCLRNCKTYQARKPLWFYNTSLKFFLNKPMLADVVFEIQGTTVPAHRAILVARCEVMAAMFNGNYMEAKSVLIPVYGVSKETFLSFLEYLYTDSCCPAGIFQAMCLLICAEMYQVSRLQHICELFIITQLQSMPSRELASMNLDIVDLLKKAKFHHSDCLSTWLLHFIATNYLIFSQKPEFQDLSVEERSFVEKHRWPSNMYLKQLAEYRKYIHSRKCRCLVM

SEQ ID NO: 15 - DNA, 846 bp, Homo sapiens
  CDS (1)..(843)
atggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaa

SEQ ID NO: 16 - protein, 281 aa, Homo sapiens [sequence omitted]

SEQ ID NO: 17 - DNA, 1407 bp, Homo sapiens
  CDS (1)..(1404)
atggcgccaccaccagctagagtacatctaggtgcgttcctggcagtgactccgaatcccgggagcgcagcgagtgggacagaggcagccgcggccacacccagcaaagtgtggggctcttccgcggggaggattgaaccacgaggcgggggccgaggagcgctccctacctccatgggacagcacggacccagtgcccgggcccgggcagggcgcgccccaggacccaggccggcgcgggaagccagccctcggctccgggtccacaagaccttcaagtttgtcgtcgtcggggtcctgctgcaggtcgtacctagctcagctgcaaccatcaaacttcatgatcaatcaattggcacacagcaatgggaacatagccctttgggagagttgtgtccaccaggatctcatagatcagaacatcctggagcctgtaaccggtgcacagagggtgtgggttacaccaatgcttccaacaatttgtttgcttgcctcccatgtacagcttgtaaatcagatgaagaagagagaagtccctgcaccacgaccaggaacacagcatgtcagtgcaaaccaggaactttccggaatgacaattctgctgagatgtgccggaagtgcagcagagggtgccccagagggatggtcaaggtcaaggattgtacgccctggagtgacatcgagtgtgtccacaaagaatcaggcaatggacataatatatgggtgattttggttgtgactttggttgttccgttgctgttggtggctgtgctgattgtctgttgttgcatcggctcaggttgtggaggggaccccaagtgcatggacagggtgtgtttctggcgcttgggtctcctacgagggcctggggctgaggacaatgctcacaacgagattctgagcaacgcagactcgctgtccactttcgtctctgagcagcaaatggaaagccaggagccggcagatttgacaggtgtcactgtacagtccccaggggaggcacagtgtctgctgggaccggcagaagctgaagggtctcagaggaggaggctgctggttccagcaaatggtgctgaccccactgagactctgatgctgttctttgacaagtttgcaaacatcgtgccctttgactcctgggaccagctcatgaggcagctggacctcacgaaaaatgagatcgatgtggtcagagctggtacagcaggcccaggggatgccttgtatgcaatgctgatgaaatgggtcaacaaaactggacggaacgcctcgatccacaccctgctggatgccttggagaggatggaagagagacatgcaaaagagaagattcaggacctcttggtggactctggaaagttcatctacttagaagatggcacaggctctgccgtgtccttggagtga

SEQ ID NO: 18 - protein, 468 aa, Homo sapiens
MAPPPARVHLGAFLAVTPNPGSAASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVHKESGNGHNIWVILVVTLVVPLLLVAVLIVCCCIGSGCGGDPKCMDRVCFWRLGLLRGPGAEDNAHNEILSNADSLSTFVSEQQMESQEPADLTGVTVQSPGEAQCLLGPAEAEGSQRRRLLVPANGADPTETLMLFFDKFANIVPFDSWDQLMRQLDLTKNEIDVVRAGTAGPGDALYAMLMKWVNKTGRNASIHTLLDALERMEERHAKEKIQDLLVDSGKFIYLEDGTGSAVSLE

SEQ ID NO: 19 - DNA, 1323 bp, Homo sapiens
  CDS (1)..(1320)
atggaacaacggggacagaacgccccggccgcttcgggggcccggaaaaggcacggcccaggacccagggaggcgcggggagccaggcctgggctccgggtccccaagacccttgtgctcgttgtcgccgcggtcctgctgttggtctcagctgagtctgctctgatcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccctgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggtacaaagcacagtggggaagccccagctgtggaggagacggtgacctccagcccagggactcctgcctctccctgttctctctcaggcatcatcataggagtcacagttgcagccgtagtcttgattgtggctgtgtttgtttgcaagtctttactgtggaagaaagtccttccttacctgaaaggcatctgctcaggtggtggtggggaccctgagcgtgtggacagaagctcacaacgacctggggctgaggacaatgtcctcaatgagatcgtgagtatcttgcagcccacccaggtccctgagcaggaaatggaagtccaggagccagcagagccaacaggtgtcaacatgttgtcccccggggagtcagagcatctgctggaaccggcagaagctgaaaggtctcagaggaggaggctgctggttccagcaaatgaaggtgatcccactgagactctgagacagtgcttcgatgactttgcagacttggtgccctttgactcctgggagccgctcatgaggaagttgggcctcatggacaatgagataaaggtggctaaagctgaggcagcgggccacagggacaccttgtacacgatgctgataaagtgggtcaacaaaaccgggcgagatgcctctgtccacaccctgctggatgccttggagacgctgggagagagacttgccaagcagaagattgaggaccacttgttgagctctggaaagttcatgtatctagaaggtaatgcagactctgccatgtcctaa

SEQ ID NO: 20 - protein, 440 aa, Homo sapiens
MEQRGQNAPAASGARKRHGPGPREARGARPGLRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEAPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 21 - DNA, 1236 bp, Homo sapiens
  CDS (1)..(1233)
atggaacaacggggacagaacgccccggccgcttcgggggcccggaaaaggcacggcccaggacccagggaggcgcggggagccaggcctgggctccgggtccccaagacccttgtgctcgttgtcgccgcggtcctgctgttggtctcagctgagtctgctctgatcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccctgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggcatcatcataggagtcacagttgcagccgtagtcttgattgtggctgtgtttgtttgcaagtctttactgtggaagaaagtccttccttacctgaaaggcatctgctcaggtggtggtggggaccctgagcgtgtggacagaagctcacaacgacctggggctgaggacaatgtcctcaatgagatcgtgagtatcttgcagcccacccaggtccctgagcaggaaatggaagtccaggagccagcagagccaacaggtgtcaacatgttgtcccccggggagtcagagcatctgctggaaccggcagaagctgaaaggtctcagaggaggaggctgctggttccagcaaatgaaggtgatcccactgagactctgagacagtgcttcgatgactttgcagacttggtgccctttgactcctgggagccgctcatgaggaagttgggcctcatggacaatgagataaaggtggctaaagctgaggcagcgggccacagggacaccttgtacacgatgctgataaagtgggtcaacaaaaccgggcgagatgcctctgtccacaccctgctggatgccttggagacgctgggagagagacttgccaagcagaagattgaggaccacttgttgagctctggaaagttcatgtatctagaaggtaatgcagactctgccatgtcctaa

SEQ ID NO: 22 - protein, 411 aa, Homo sapiens
MEQRGQNAPAASGARKRHGPGPREARGARPGLRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 23 - DNA, 735 bp, Artificial
  Humanized anti-human TRAILR1 scFv antibody
gaggtccagctggtacagtctggagctgaagtgaagatgcctggggcctcagtcaagctctcctgcagggtttctggagacaccttcaccgcctacttcattcactgggtgcgacaggcccctggacaagggcttgagtggatgggatggttcaaccctatcagtggcaccgcaggctctgctgagaagtttcgcggcagggtcgccatgaccagggacacgtccatcagcactgcctacatggaattgaacaggctgacatttgacgacacggccgtctattattgtgcgagacaacatcgggggaatacgtttgacccctggggccagggcaccctggtcaccgtctcgagtggaggcggcggttcaggcggaggtggctctggcggtggcggaagtgcacagtctgccctgactcagcctgcctccgtgtctgggtctcctggacagtcgatcaccatctcctgcactggaaccagcagtgacattggtgcttataagtatgtctcctggtatcaacaacacccaggcaaagcccccaaacttgtgatttatgaggtcagtaatcggccctcaggggtttccagtcgcttctctggctccaagtctggccagacggcctccctgaccatctctgggctccaggctgacgacgaggctgattattactgcaactcatatcaaggttacaacacgtgggtgttcggcggagggaccaaggtcaccgtcctaggt

SEQ ID NO: 24 - protein, 245 aa, Artificial
  Synthetic Construct
EVQLVQSGAEVKMPGASVKLSCRVSGDTFTAYFIHWVRQAPGQGLEWMGWFNPISGTAGSAEKFRGRVAMTRDTSISTAYMELNRLTFDDTAVYYCARQHRGNTFDPWGQGTLVTVSSGGGGSGGGGSGGGGSAQSALTQPASVSGSPGQSITISCTGTSSDIGAYKYVSWYQQHPGKAPKLVIYEVSNRPSGVSSRFSGSKSGQTASLTISGLQADDEADYYCNSYQGYNTWVFGGGTKVTVLG